(19) United States
(12) Patent Application Publication
(10) Pub. No.: US 2023/0172610 A1
(43) Pub. Date: Jun. 8, 2023

(54) COMBINATORIAL THERAPIES INCLUDING IMPLANTABLE DAMPING DEVICES AND BIOLOGIC THERAPEUTIC AGENTS FOR TREATING A CONDITION AND ASSOCIATED SYSTEMS AND METHODS OF USE

(71) Applicant: CARNEGIE VENTURE CAPITAL PTY LIMITED, Paddington (AU)

(72) Inventors: David Stephen Celermajer, Vaucluse (AU); Zoran Milijasevic, Bayview (AU); Anthony John Ujhazy, East Lindfield (AU); Mark Carnegie, Takaka (NZ); Rachel Ashley Levin, North Sydney (AU)

(21) Appl. No.: 17/916,777

(22) PCT Filed: Apr. 2, 2021
(86) PCT No.: PCT/IB2021/000218

§ 371 Date: Oct. 3, 2022

Related U.S. Application Data
(60) Provisional application No. 63/004,375, filed on Apr. 2, 2020.

(51) Int. Cl. A61B 17/12; A61K 35/28; A61P 25/28; A61K 48/00; A61K 35/741; A61K 38/22; A61K 38/45

(52) U.S. Cl. CPC A61B 17/12036; A61B 17/12109; A61B 17/12186; A61K 35/28; A61K 35/741; A61K 38/45; A61K 38/2285; A61K 48/005; A61P 25/28; A61B 2017/00893

(57) ABSTRACT

Devices, systems, and methods for combinatorial treatment of a condition with an implantable damping device and biologic therapeutic agent are disclosed herein. Methods for treating one or more effects of the condition, such as a neurological condition, include providing the implantable damping device and at least one other therapy, such as a biologic therapeutic agent, that treats the condition to the patient. The implantable damping device includes a flexible damping member and an abating substance and can be placed in apposition with a blood vessel. The flexible damping member forms a generally tubular structure having an inner and an outer surface, the inner surface formed of a sidewall having a partially deformable portion. The abating substance is disposed within the partially deformable portion and moves longitudinally and/or radially within the partially deformable portion in response to pulsatile blood flow.

### CROSS-REFERENCE TO RELATED APPLICATION(S)

This application claims priority to U.S. Pat. Application No. 63/004,375, entitled “COMBINATORIAL THERAPIES INCLUDING IMPLANTABLE DAMPING DEVICES AND BIOLOGIC THERAPEUTIC AGENTS FOR TREATING A CONDITION AND ASSOCIATED SYSTEMS AND METHODS OF USE,” filed on Apr. 2, 2020, which is herein incorporated by reference in its entirety.

### TECHNICAL FIELD

The present technology relates to combinatorial therapies including an implantable damping device and biologic therapeutic agents for treating a condition (e.g., a neurodegenerative condition such as dementia) and associated systems and methods of use. In particular, the present technology is directed to combinatorial therapies including an implantable damping device for positioning at, near, within, around, or in place of at least a portion of an artery and one or more biologic therapeutic agents (e.g., cell-based, gene-based, and microbiome-based agents) for treating the condition.

### BACKGROUND

The heart supplies oxygenated blood to the body through a network of interconnected, branching arteries starting with the largest artery in the body—the aorta. As shown in the schematic view of the heart and selected arteries in FIG. 1A, the portion of the aorta closest to the heart is divided into three regions: the ascending aorta (where the aorta initially leaves the heart and extends in a superior direction), the aortic arch, and the descending aorta (where the aorta extends in an inferior direction). Three major arteries branch from the aorta along the aortic arch: the brachiocephalic artery, the left common carotid artery, and the left subclavian artery. The brachiocephalic artery extends away from the aortic arch and subsequently divides into the right common carotid artery, which supplies oxygenated blood to the head and neck, and the right subclavian artery, which predominantly supplies blood to the right arm. The left common carotid artery extends away from the aortic arch and supplies the head and neck. The left subclavian artery extends away from the aortic arch and predominantly supplies blood to the left arm. Each of the right common carotid artery and the left common carotid artery subsequently branches into separate internal and external carotid arteries.

During the systole stage of a heartbeat, contraction of the left ventricle forces blood into the ascending aorta that increases the pressure within the arteries (known as systolic blood pressure). The volume of blood ejected from the left ventricle creates a pressure wave-known as a pulse wave-that propagates through the arteries propelling the blood. The pulse wave causes the arteries to dilate, as shown schematically in FIG. 1B. When the left ventricle relaxes (the diastole stage of a heartbeat), the pressure within the arterial system decreases (known as diastolic blood pressure), which allows the arteries to contract.

The difference between the systolic blood pressure and the diastolic blood pressure is the “pulse pressure,” which generally is determined by the magnitude of the contraction force generated by the heart, the heart rate, the peripheral vascular resistance, and diastolic “run-off” (e.g., the blood flowing down the pressure gradient from the arteries to the veins), amongst other factors. High flow organs, such as the brain, are particularly sensitive to excessive pressure and flow pulsatility. To ensure a relatively consistent flow rate to such sensitive organs, the walls of the arterial vessels expand and contract in response to the pressure wave to absorb some of the pulse wave energy. As the vasculature ages, however, the arterial walls lose elasticity, which causes an increase in pulse wave speed and wave reflection through the arterial vasculature. Arterial stiffening impairs the ability of the carotid arteries and other large arteries to expand and dampen flow pulsatility, which results in an increase in systolic pressure and pulse pressure. Accordingly, as the arterial walls stiffen over time, the arteries transmit excessive force into the distal branches of the arterial vasculature.

Research suggests that consistently high systolic pressure, pulse pressure, and/or change in pressure over time (dP/dt) increases the risk of dementia, such as vascular dementia (e.g., an impaired supply of blood to the brain or bleeding within the brain). Without being bound by theory, it is believed that high pulse pressure can be the root cause or an exacerbating factor of vascular dementia and age-related dementia (e.g., Alzheimer’s disease). As such, the progression of vascular dementia and age-related dementia (e.g., Alzheimer’s disease) may also be affected by the loss of elasticity in the arterial walls and the resulting stress on the cerebral vessels. Alzheimer’s Disease, for example, is generally associated with the presence of neuritic plaques and tangles in the brain. Recent studies suggest that increased pulse pressure, increased systolic pressure, and/or an increase in the rate of change of pressure (dP/dt) may, over time, cause microbleeds within the brain that may contribute to the neuritic plaques and tangles.

By 2050, it is estimated that at least one in every 85 people will be living with Alzheimer’s disease world-wide and more than eight times as many people have shown preclinical symptoms. Additional disease-modifying therapies that will prevent or delay the onset or slow progression of neurological conditions, such as dementia, have been and are being developed. As of March 2020, there are 2,272 clinical trials and/or other related testing ongoing for treatment of Alzheimer’s disease, one of several neurological conditions that is becoming increasingly more common as the world’s population ages. While the therapeutic agents undergoing testing in these clinical trials may improve memory, behavior, cognition and/or reduce neuropsychiatric symptoms of Alzheimer’s disease, additional studies testing the efficacy, safety, and tolerability of these therapeutic agents, and/or use of additional therapeutic agents are needed. Accordingly, there is a need for improved devices, systems, and methods for treating vascular and/or age-related dementia.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a schematic illustration of a human heart and a portion of the arterial system near the heart.

FIG. 1B is a schematic illustration of a pulse wave propagating along a blood vessel.

FIG. 2A is a front view of a damping device in accordance with the present technology, shown in a deployed, relaxed state.

FIG. 2B is a front cross-sectional view of the damping device shown in FIG. 2A.

FIG. 2C is a front cross-sectional view of the damping device shown in FIG. 2A, shown in a deployed state positioned within a blood vessel.

FIG. 2D is a front cross-sectional view of another embodiment of a damping device in accordance with the present technology, shown in a deployed, relaxed state.

FIGS. 2E-2G are front cross-sectional views of several embodiments of damping members in accordance with the present technology, all shown in a deployed, relaxed state.

FIG. 3A is a front cross-sectional view of another embodiment of a damping device in accordance with the present technology shown in a deployed, relaxed state.

FIGS. 3B-3D are front cross-sectional views of several embodiments of damping members in accordance with the present technology, all shown in a deployed, relaxed state.

FIG. 4A is a front view of a damping device in accordance with another embodiment of the present technology, shown in a deployed, relaxed state.

FIG. 4B is a front cross-sectional view of the damping device shown in FIG. 4A.

FIG. 4C is a front cross-sectional view of the damping device shown in FIG. 4A, shown in a deployed state positioned within a blood vessel.

FIG. 4D is a front cross-sectional view of a portion of a damping member in accordance with the present technology showing deformation of the damping member (in dashed lines) in response to a pulse wave.

FIG. 4E is a front cross-sectional view of a portion of another damping member in accordance with the present technology showing deformation of the damping member (in dashed lines) in response to a pulse wave.

FIGS. 5-7 are front cross-sectional views of several embodiments of damping devices in accordance with the present technology.

FIGS. 8A-8E illustrate a method of delivering a damping device to an artery in accordance with the present technology.

FIGS. 9A-9F are schematic cross-sectional views of several embodiments of damping members in accordance with the present technology.

FIGS. 10 and 11 are front cross-sectional views of embodiments of damping devices shown positioned at or near a resected blood vessel in accordance with the present technology.

FIG. 12A is a front view of a helical damping device in accordance with the present technology, shown positioned around a blood vessel in a deployed, relaxed state.

FIG. 12B is a cross-sectional view of the damping device of FIG. 12A (taken along line 12B-12B in FIG. 12A), shown positioned around the blood vessel as a pulse pressure wave travels through the vessel.

FIGS. 13 and 14 show different embodiments of a wrapped damping device, each shown positioned around a blood vessel in accordance with the present technology.

FIG. 15 is a cross-sectional view of another embodiment of a damping device in accordance with the present technology.

FIG. 16A is a perspective view of another embodiment of a damping device in accordance with the present technology.

FIG. 16B is a cross-sectional view of the damping device shown in FIG. 16A, taken along line 16B-16B.

FIG. 17A is a perspective view of another embodiment of a damping device in accordance with the present technology.

FIG. 17B is a cross-sectional view of the damping device shown in FIG. 17A.

FIG. 18A is a perspective view of another embodiment of a damping device in accordance with the present technology.

FIG. 18B is a front view of the damping device shown in FIG. 18A, shown in a deployed state positioned around a blood vessel.

FIG. 19A is a perspective view of a damping device in accordance with another embodiment of the present technology, shown in an unwrapped state.

FIG. 19B is a top view of the damping device shown in FIG. 19A, shown in an unwrapped state.

FIG. 20 is a flow chart illustrating a method in accordance with the present technology.

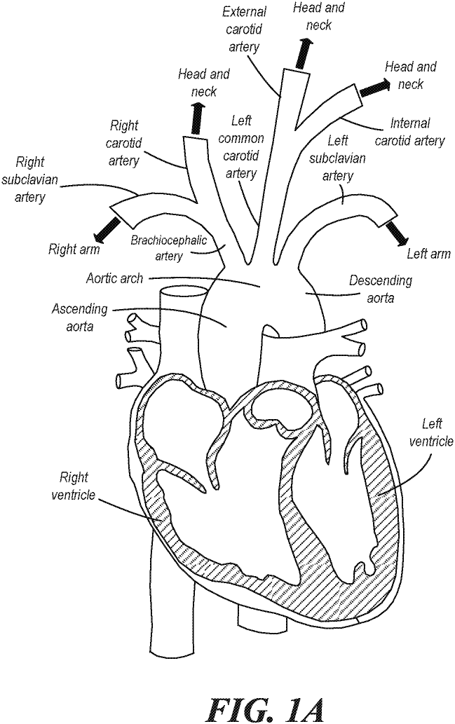

### DETAILED DESCRIPTION

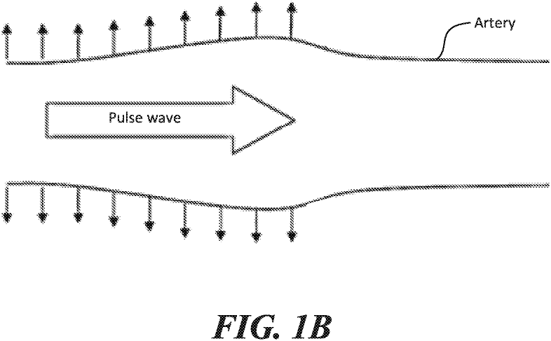

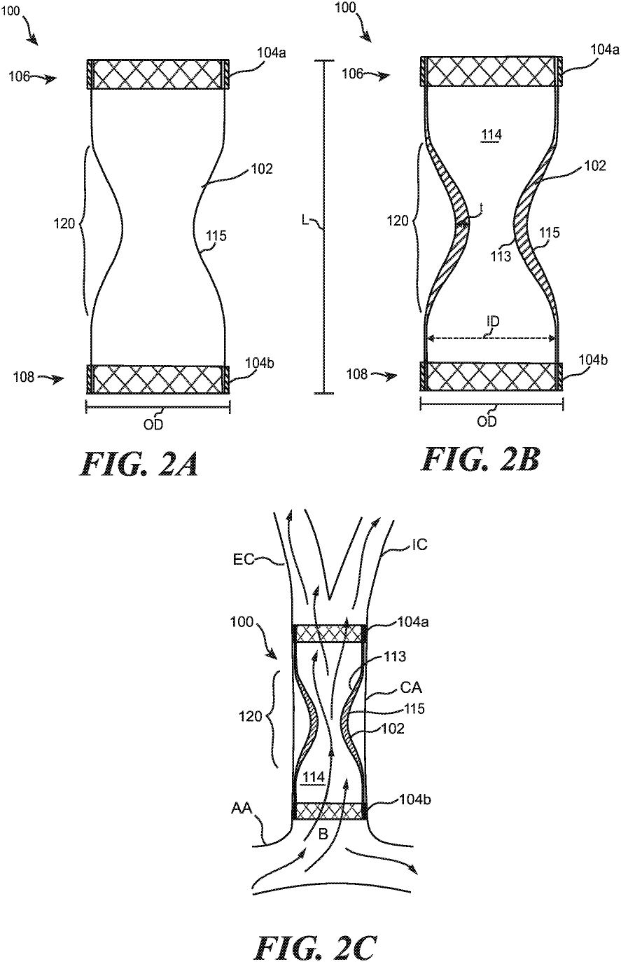

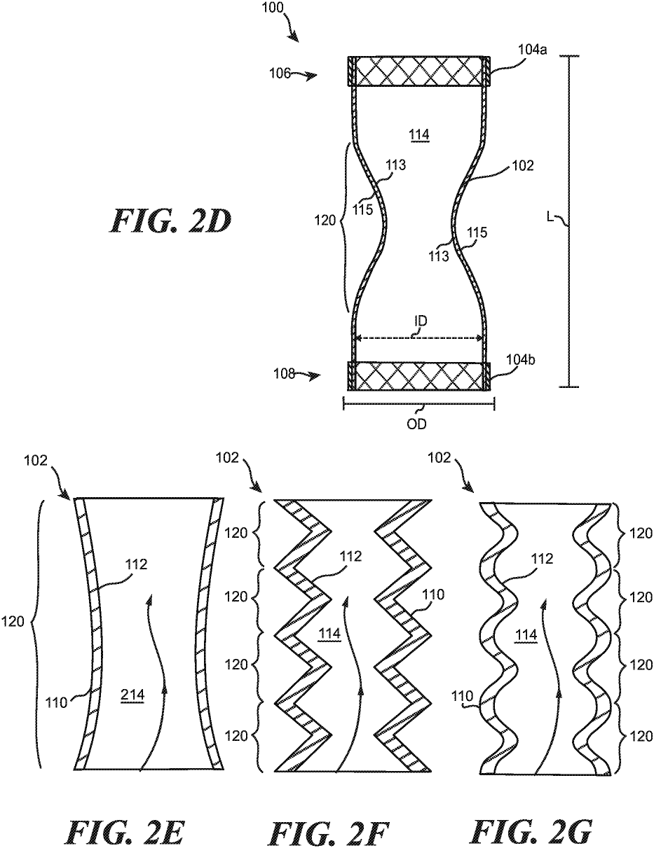

The present technology is directed to combinatorial therapies including an implantable damping device and a biologic therapeutic agent (e.g., a cell-based, gene-based, a peptide-based, and/or microbiome-based biologic therapy) for treating and/or preventing the progression of a condition, including neurological conditions such as dementia (e.g., vascular dementia and age-related dementia), and associated systems and methods of use. Some embodiments of the present technology, for example, are directed to combinatorial device and biologic therapies including damping devices having an anchoring member and a flexible, compliant damping member having an outer surface and an inner surface defining a lumen configured to direct blood flow. The inner surface is configured such that a cross-sectional dimension of the lumen varies. For example, the outer surface and the inner surface can be separated from each other by a distance that varies along the length of the damping member. The damping member can further include a first end portion, a second end portion opposite the first end portion, and a damping region between the first and second end portions. The distance between the outer surface and the inner surface of the damping member can be greater at the damping region than at either of the first or second end portions. When blood flows through the damping member during systole, the damping member absorbs a portion of the pulsatile energy of the blood to reduce the magnitude of the pulse pressure transmitted to a portion of the blood vessel distal to the damping device. Additional embodiments of the present technology, for example, are directed to combinatorial device and biologic therapies including biologic therapeutic agents (e.g., a cell-based, gene-based, peptide-based, and/or microbiome-based biologic therapy) that have been developed or are currently being developed to treat or otherwise slow the effects of neurological conditions. These biologic therapeutic agents, and other biologic therapeutic agents derived from and/or otherwise based upon these biologic therapeutic agents, are included in embodiments of the present technology. Specific details of several embodiments of the technology are described below with reference to FIGS. 2A-20.

With regard to the terms “distal” and “proximal” within this description, unless otherwise specified, the terms can reference a relative position of the portions of a damping device and/or an associated delivery device with reference to an operator, direction of blood flow through a vessel, and/or a location in the vasculature. For example, in referring to a delivery catheter suitable to deliver and position various damping devices described herein, “proximal” refers to a position closer to the operator of the device or an incision into the vasculature, and “distal” refers to a position that is more distant from the operator of the device or further from the incision along the vasculature (e.g., the end of the catheter).

As used herein, “artery” and “arteries that supply blood to the brain,” include any arterial blood vessel (or portion thereof) that provides oxygenated blood to the brain. For example, “arteries” or “arteries that supply blood to the brain” can include the ascending aorta, the aortic arch, the brachiocephalic trunk, the right common carotid artery, the left common carotid artery, the left and right internal carotid arteries, the left and right external carotid arteries, and/or any branch and/or extension of any of the arterial vessels described above.

As used herein, the terms “biologic therapeutic agent”, “biologic agent”, “biologic therapy”, and “biologic therapies” are used interchangeably within this description to refer to a cell-based therapeutic agent, gene-based therapeutic agent or a gene product produced therefrom, a peptide-based therapeutic agent, and/or a microbiome-based therapy. Biologic agents and therapies may be composed of a wide variety of substances including, but not limited to, sugars (or other biologic small molecules), proteins or peptides, nucleic acids, complex combinations of those substances, or may be living entities such as cells or tissues. Biologic agents and therapies include a wide variety of products and treatments that may be isolated from or otherwise derived from natural sources (e.g., human, animal, plant, microorganism) using recombinant, synthetic, or other biotechnology methods and technologies. For example, a peptide or other natural molecule that is synthetically produced may be considered a biologic agent in accordance with this disclosure.

The terms “cell-based therapeutic agent” and “cell-based therapy” are used interchangeably within this description to refer to therapeutic agents and/or therapies involving stem cells, progenitor cells or precursor cells, extracellular vesicles, and genetically modified immune cells. Stem cells include, but are not limited to, self-renewing, totipotent, pluripotent, and/or multipotent cells. Examples of stem cells include, but are not limited to, embryonic stem cells, naive stem cells, somatic stem cells, adult stem cells, mesenchymal stem cells, hematopoietic stem cells, neural stem cells, induced pluripotent stem cells, and genetically modified stem cells. Markers (e.g., peptides) expressed by stem cells include, but are not limited to, SSEA-4, TRA-1-60, TRA-1-81, OCT4, SOX2, CD90, CD34, CD133, CD73, and CD105. Progenitor cells or precursor cells are cells which are not fully differentiated, but also not totipotent or pluripotent. Examples of progenitor or precursor cells include, but are not limited to, endothelial progenitor cells and neural progenitor cells. Extracellular vesicles are lipid-bilayer delimited and released from any type of cell. Examples of extracellular vesicles include, but are not limited to, stem cell derived microvesicles, exosomes, and ectosomes. Genetically modified immune cells are white blood cells genetically engineered to express a non-native peptide, such as a peptide not normally expressed by a white blood cell, including but not limited to wild-type peptides and synthetic peptides. Examples of genetically modified immune cells include, but are not limited to, T cells and/or phagocytes which target peptides associated with Alzheimer’s disease, such as Alzheimer’s biomarkers including, but not limited to, β-amyloid and/or brain-derived neurotrophic factor (BDNF). The BDNF may be secreted by the genetically modified immune cell.

The terms “gene-based therapeutic agent”, “gene-based therapy”, and “gene therapy” are used interchangeably within this description to refer to therapeutic agents and/or therapies involving modifying expression of a gene in a host (e.g., a cell, a subject). Examples of modification include transferring genetic material to the host and inducing its expression within the host, causing expression of the modified genetic material (e.g., a peptide, a protein, or a portion thereof) and/or reducing, preventing, or otherwise eliminating expression of genetic material (e.g., DNA or RNA) within the host. Such modifications can be performed in the host cell by introducing a gene, DNA, or RNA transcript into the host cell, augmenting expression or regulation of a gene, suppressing expression of a gene, and/or editing at least a portion of the host’s genome (e.g., using TALENs, zinc-finger nucleases, CRISPR, or the like). Therapeutic agents useful in gene-based therapy include transgenes carried by viral vectors and nonviral vectors. A viral vector refers to a virus particle engineered to genetically modify a host (e.g., cell, such as a mammalian cell) in vivo or ex vivo. Types of viruses that viral vectors can be isolated from, or derived from, and otherwise modified include, but are not limited to, adeno-associated virus (AAV), adenovirus, lentivirus, and retrovirus. A nonviral vector refers to a nucleic acid that is bare and packaged using a delivery system rather than carried by a viral vector. Bare nucleic acids include DNA and RNA. Suitable delivery systems for packaging include (1) vesicles and nanoparticles, such as but not limited to, exosomes, lipoplexes, polyplexes, and gold nanoparticles; (2) carrier compounds, including but not limited to, DNA-binding carrier proteins, and (3) bactofection.

The terms “microbiome-based therapy,” “microbiome-based therapy,” and “microbiome therapy” are used interchangeably within this description to refer to therapeutic agents and/or therapies involving delivery of one or more microbiome agents to a host. Microbiome agents include, but are not limited to, microbiome transplants, probiotics, phages, and a drug. Microbiome transplant refers generally to methods of introducing healthy microbiome material to a host including, but not limited to, fecal bacteriotherapy and fecal microbiota transplant. Probiotics refers to one or more isolated microbial species that are delivered to a host including, but not limited to, omni-biotic stress repair, a characterized bacterium, and/or a characterized bacterial strain or combination of characterized bacterial strains. Phages and associated phage-based microbiome therapy refers to reducing or elimination of one or more detrimental bacteria in a host by delivering one or more bacteriophages to the host. An example bacteriophage useful for phage-based microbiome therapy includes P. gingivalis. Drugs useful for microbiome-based therapy are biologic and/or chemical compounds that alter a composition of a host microbiome, metabolism of the host microbiome, and signaling events involving the host microbiome. An example drug is GV-971.

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The terms “peptide,” “polypeptide,” and “protein” are used interchangeably to refer to a polymer of amino acid residues, and are not limited to a minimum length, though a number of amino acid residues may be specified (e.g., 9mer is nine amino acid residues). Polypeptides may include amino acid residues including natural and/or non-natural amino acid residues. Polypeptides may also include fusion proteins. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, such as through site-directed mutagenesis, or may be accidental, such as through mutations of hosts which produce the proteins or errors due to PCR amplification.

The term “derivative” refers to a molecule having a sequence that has at least 80% of the nucleotides or amino acids in the same sequence as the molecule to which the term derivative modifies. Exemplary derivatives are peptide derivatives which have an amino acid sequence that includes at least 80% of the amino acid sequence of the non-derivative peptide and nucleic acid derivatives which have a nucleic acid sequence that includes at least 80% of the nucleic sequence of the non-derivative nucleic acid.

The term “variant” refers to a molecule having a post-translational modification that does not alter the amino acid sequence (e.g., if the molecule is a peptide) or an epigenetic modification that does not alter the nucleic acid sequence (e.g., if the molecule is a nucleic acid) compared to a non-variant peptide or non-variant nucleic acid. Exemplary variants are peptides having nitrosylation, acylation, palmitoylation, and/or other post-translational modifications and nucleic acids having methylcytosine (SmC), 5-hydroxymethylcytosine (5hmC), 5-formylcytosine (5fC), 5-carboxycytosine (ScaC), N6-methyladenosine (m6A) modifications, and/or other epigenetic modifications.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L, and norleucine (Nle).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and poly deoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN, or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “gene” refers to a nucleic acid (e.g., DNA) sequence that comprises coding sequences necessary for the production of a polypeptide, precursor, or RNA (e.g., rRNA, tRNA, siRNA, long non-coding RNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (linRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to any gene that is introduced into an organism (e.g., a human or a non-human animal). Transgenes are nucleic acids that encode a non-native gene, an extra copy of an endogenous gene, or disrupt expression of a wild-type gene that include, but are not limited to, nerve growth factor (NGF), telomerase reverse transcriptase (hTERT), apolipoprotein E2 (APOE2), β-arrestin-2, genes encoding at least a portion of an antibody, a neurotrophic factor, a CRISPR-Cas9 cassette, zinc finger nuclease/zinc finger technology, a microRNA, and an siRNA. As used herein, the term “transgenic organism” refers to an organism that expresses a transgene.

As used herein, the term “gene expression” refers to the process of converting genetic information encoded in a gene into RNA (e.g., mRNA, microRNA, lncRNA, siRNA rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source and/or a recombinant gene having a wild-type sequence, a native sequence or a sequence otherwise the same as a wild-type sequence despite not being isolated form a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product. As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence. As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements. The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded) but may contain both the sense and anti-sense strands (i.e., the oligonucleotide or polynucleotide may be double-stranded).

The terms “overexpression” and “overexpressing” and grammatical equivalents are used in reference to levels of mRNA approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA. The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, viral infection, and biolistics. The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol, 52:456 [1973]), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA. The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA. As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non- dominant selectable markers include the thymidine kinase (tk) gene that is used in conjunction with tk - cell lines, the CAD gene that is used in conjunction with CAD-deficient cells and the mammalian hypoxanthine-guanine phosphoribosyl transferase (hprt) gene that is used in conjunction with hprt - cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

The term “isolated” means that the material is removed from its original environment (e.g., the natural environment if it is naturally occurring). Such nucleic acid could be part of a vector and/or such nucleic acid or polypeptide could be part of a composition (e.g., a cell lysate), and still be isolated in that such vector or composition is not part of the natural environment for the nucleic acid or polypeptide.

As used herein, the terms “homologous,” “homology,” or “percent homology” when used herein to describe a nucleic acid sequence relative to a reference sequence, can be determined using the formula described by Karlin & Altschul 1990, modified as in Karlin & Altschul 1993. Such a formula is incorporated into the basic local alignment search tool (BLAST) programs of Altschul 1990. Percent homology of sequences can be determined using the most recent version of BLAST, as of the filing date of this application.

A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound. In certain embodiments, a functional portion refers to a “signaling portion” of an effector molecule, effector domain, costimulatory molecule, or costimulatory domain.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

As used herein, “expression vector” refers to a nucleic acid construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a linear nucleic acid. Once transformed into a suitable host, the vector may replicate and function independently of the host genome, or may, in some instances, integrate into the genome itself. Here, “plasmid,” “expression plasmid,” “virus,” and “vector” are often used interchangeably.

The term “introduced” in the context of inserting a nucleic acid molecule into a cell means “transfection,” “transformation,” or “transduction” and includes reference to the incorporation of a nucleic acid molecule into a eukaryotic cell wherein the nucleic acid molecule may be incorporated into the genome of a cell and converted into an autonomous replicon. As used herein, the term “engineered,” “recombinant,” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, CARs or enzymes, or other nucleic acid molecule additions, deletions, substitutions, or other functional disruption of a cell’s genetic material.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related, or unrelated to, biosynthesis of the heterologous protein.

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. In certain embodiments, amounts of a certain cell type in a mixture will be enriched and amounts of a different cell type will be depleted, such as enriching for CD4+ cells while depleting CD8+ cells, or enriching for CD8+ cells while depleting CD4+ cells, or combinations thereof.

“Antigen” as used herein refers to an immunogenic molecule that provokes an immune response, such as an epitope which includes any molecule, structure, amino acid sequence, or protein determinant that is recognized and specifically bound by a cognate binding molecule, such as an antibody, chimeric antigen receptor, or other binding molecule, domain, or protein. This immune response may involve antibody production, activation of specific immunologically-competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

“Exogenous” with respect to a nucleic acid or polynucleotide indicates that the nucleic acid is part of a recombinant nucleic acid construct or is not in its natural environment. For example, an exogenous nucleic acid can be a sequence from one species introduced into another species (i.e., a heterologous nucleic acid). Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, for example, non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example, using genetic recombination. Genetic recombination is the breaking and rejoining of DNA strands to form new molecules of DNA encoding a novel set of genetic information.

With regard to the term “neurological condition” within this description, unless otherwise specified, the term refers to a condition, a disorder, and/or a disease of the brain, spine, and nerves connecting the brain and the spine. Neurological conditions include, but are not limited to dementia (e.g., vascular, frontotemporal, Lewy body), Alzheimer’s disease, Huntington’s disease, cognitive impairment, Parkinson’s disease, neuralgia, tumor, cancer, stroke, aneurysm, epilepsy, headache, and/or migraine.

A “subject in need thereof” as used herein refers to a mammalian subject, preferably a human, who has been diagnosed with a neurologic condition, is suspected of having a neurologic condition, and/or exhibits one or more symptoms or risk factors associated with a neurologic condition.

The terms “treating” and “treatment” in relation to a given condition, disease, or disorder are used interchangeably and include, but are not limited to, inhibiting the disease or disorder, for example, arresting the development or rate of development of the condition, disease, or disorder; relieving the condition, disease, or disorder, for example, causing regression of the condition, disease, or disorder; or relieving a condition caused by or resulting from the disease or disorder, for example, arresting, relieving, preventing, or causing regression of at least one of the symptoms of the disease or disorder.

The terms “preventing” and “prevention” in relation to a given condition, disease, or disorder are used interchangeably and include, but are not limited to, preventing or delaying the onset of its development if none had occurred; preventing or delaying the condition, disease, or disorder from occurring in a subject that may be predisposed to the condition, disease, or disorder but has not yet been diagnosed as having the condition, disease; or disorder, and/or preventing or delaying further development of the condition, disease, or disorder if already present.

As used herein, “route” in relation to administration of one or more therapies, such as a therapeutic agent (e.g., drug), refers to a path by which the therapeutic agent is delivered to a subject, for example, a subject’s body. A route of therapeutic administration include enteral and parenteral routes of administration. Enteral administration includes oral, rectal, intestinal, and/or enema. Parenteral includes topical, transdermal, epidural, intracerebral, intracerebroventricular, epicutaneous, sublingual, sublabial, buccal, inhalational (e.g., nasal), intravenous, intraarticular, intracardiac, intradermal, intramuscular, intraocular, intraosseous infusion, intraperitoneal, intrathecal, intravitreal, subcutaneous, perivascular, implantation, vaginal, otic, and/or transmucosal.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein is to be understood to include any integer within the recited range, unless otherwise indicated. As used herein, the term “about” means ± 20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include, but not be limited to, systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense that one having skill in the art would understand the convention (e.g., “a system having at least one of A, B, or C” would include, but not be limited to, systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting. Further, headings provided herein are for convenience only and do not interpret the scope or meaning of the claimed embodiments.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

While the present invention is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

### I. Selected Intravascular Embodiments of Damping Devices

FIGS. 2A and 2B are a front view and a front cross-sectional view, respectively, of a damping device 100 configured in accordance with the present technology shown in an expanded or deployed state. FIG. 2C is a front view of the damping device 100 in a deployed state positioned in a carotid artery CA (e.g., the left or right carotid artery). Referring to FIGS. 2A-2C together, the damping device 100 includes a flexible, viscoelastic damping member 102 (e.g., a cushioning member) and anchoring members 104 (identified individually as first and second anchoring members 104a and 104b, respectively). The damping member 102 includes an undulating or hourglass-shaped sidewall having an outer surface 115 and an inner surface 113 (FIGS. 2B and 2C) that defines a lumen 114 configured to receive blood flow therethrough. The outer surface 115 is separated from the inner surface 113 by a distance t (FIG. 2B). The damping member 102 has a length L, a first end portion 106, and a second end portion 108 opposite the first end portion 106 along its length L, and a damping region 120 between the first end portion 106 and the second end portion 108. In the embodiment shown in FIGS. 2A-2C, the distance t between the outer and inner surfaces 115 and 113 varies along the length L of the damping member 102 when it is in a deployed, relaxed state. In some embodiments, the distance t between the outer and inner surfaces 115 and 113, on average, can be greater at the damping region 120 than at either of the first or second end portions 106, 108. In other embodiments, the damping member 102 can have other suitable shapes (for example, FIGS. 2E-2G), sizes, and/or configurations. For example, as shown in FIG. 2D, the distance t between the outer and inner surfaces 115 and 113 may be generally constant along the length of the damping member 102 and/or the damping region 120 when the damping member 102 is in a deployed, relaxed state.

The damping member 102 shown in FIGS. 2A-2C is a solid piece of material that is molded, extruded, or otherwise formed into the desired shape. The damping member 102 can be made of a biocompatible, compliant, viscoelastic material that is configured to deform in response to local fluid pressure in the artery. As the damping member 102 deforms, the damping member 102 absorbs a portion of the pulse pressure. The damping member 102, for example, can be made of a biocompatible synthetic elastomer, such as silicone rubber (VMQ), Tufel I and Tufel III elastomers (GE Advanced Materials, Pittsfield, MA), Sorbothane ® (Sorbothane, Incorporated, Kent, OH), and others. The damping member 102 can be flexible and elastic such that the inner diameter ID of the damping member 102 at the damping region 120 increases as a systolic pressure wave propagates through the damping region 120. For example, a systolic pressure wave may push the inner surface 113 radially outwardly, thus forcing a portion of the outer surface 115 to also deform radially outwardly. Additionally, the damping member 102 can also optionally be compressible such that the distance t between the inner and outer surfaces 115 and 113 decreases to further open the inner diameter ID of the damping region 120 as the systolic pressure wave engages the damping region 120. For example, a systolic pressure wave may push the inner surface 113 radially outwardly while the contour of the outer surface 115 remains generally unaffected.

In the embodiment shown in FIGS. 2A-2C, the anchoring members 104a-104b individually comprise a generally cylindrical structure configured to expand from a low-profile state to a deployed state in apposition with the blood vessel wall. Each of the anchoring members 104a-b can be a stent formed from a laser cut metal, such as a superelastic material (e.g., Nitinol) or stainless steel. All or a portion of each of the anchoring members can include a radiopaque coating to improve visualization of the device during delivery, and/or the anchoring members may include one or more radiopaque markers. In other embodiments, the individual anchoring members 104a-104b can comprise a mesh or woven (e.g., a braid) construction in addition to or in place of a laser cut stent. For example, the individual anchoring members 104a-104b can include a tube or braided mesh formed from a plurality of flexible wires or filaments arranged in a diamond pattern or other configuration. In some embodiments, all or a portion of one or both of the anchoring members 104a-104b can be covered by a graft material (such as Dacron) to promote sealing with the vessel wall. Additionally, all or a portion of one or both anchoring members can include one or more biomaterials.

In the embodiment shown in FIGS. 2A-2B, the anchoring members 104a-104b are positioned around the damping member 102 at the first and second end portions 106, 108, respectively. As such, in this embodiment, the outer diameter OD of the damping member 102 is less than the inner diameter of the anchoring members 104a-104b. Also in the embodiment shown in FIGS. 2A-2B, the anchoring members 104a-104b are positioned around the damping member 102 only at the first and second end portions 106, 108, respectively. As such, in several embodiments of the present technology, the damping region 120 of the damping member 120 is not surrounded by a stent-like structure or braided material. In other embodiments, the anchoring members 104 and damping member 102 may have other suitable configurations. For example, the anchoring members 104a-104b may be positioned at other locations along the length L of the damping member 102, though not along the full length of the damping member 102. Also, in some embodiments, all or a portion of one or both anchoring members 104a-104b may be positioned radially outwardly of all or a portion of the damping member 102. Although the damping device 100 shown in FIGS. 2A-2B includes two anchoring members 104a-104b, in other embodiments the damping device 100 can have more or fewer anchoring members (e.g., one anchoring member, three anchoring members, four anchoring members, etc.).

In some embodiments, a biocompatible gel or liquid may be located between the wall of the artery A and the outer surface 115 of the damping member 102 to prevent the ingression of blood into the void defined between the first anchoring member 104a, the second anchoring member 104b, the damping member 102, and the inner wall of the artery CA. Alternatively, air or another gas may be located between the internal wall of the carotid artery CA and the damping member 102 to prevent the ingression of blood into the void.

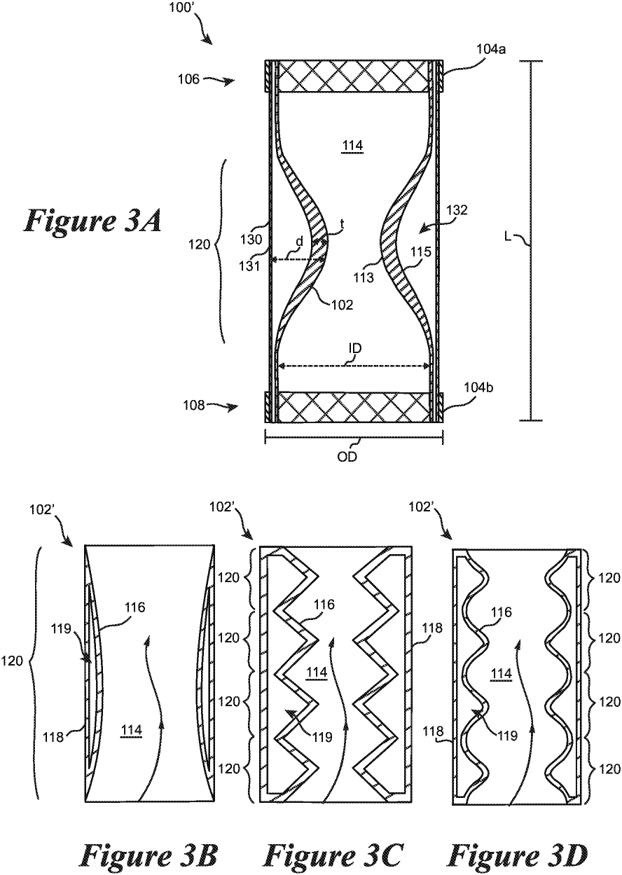

FIG. 3A is a front cross-sectional view of another embodiment of a damping device 100′ in accordance with the present technology. The embodiment of the damping device 100′ shown in FIG. 3A is similar to the embodiment of the damping device 100 shown in FIGS. 2A-2C, and like reference numbers refer to like components in FIGS. 2A-2C and FIG. 3A. As shown in FIG. 3A, the damping device 100′ includes an inner damping member 102 and an outer layer 130 surrounding the damping member 102. The outer layer 130 has an outer surface 131 and, in the embodiment shown in FIG. 3A, the first and second anchoring members 104a-b are attached to the outer surface 131. At least along the damping region 120, the outer layer 130 is spaced apart from the outer surface 115 of the damping member 102 to form a chamber 132. The chamber 132 can be at least partially filled with a fluid, such as a gas, liquid, or gel. The device 100′ has a length L and a distance d between the outer surface 131 of the outer layer 130 and the inner surface 113 of the damping member 102. Along the damping region 120, the distance d between the outer and inner surfaces 131 and 113 increases then decreases in a radial direction when the damping member 102 is in a deployed, relaxed state. On average, the distance d between the outer surface 131 and the inner surface 113 of the damping member 102 is greater at the damping region 120 than at either of the first or second end portions 106, 108. As a result, the diameter ID of the lumen 114 varies along the length L. For example, the outer surface 131 and/or the outer layer 130 can be generally cylindrical in an unbiased state, and the inner surface 113 and/or the damping member 102 can have an undulating or hourglass shape. In other embodiments, the outer surface 131 and/or the outer layer 130 can be other suitable shapes, and the inner surface 113 and/or the damping member 102 can be other suitable shapes (FIGS. 3B-3D).

In some embodiments, instead of the damping device 100′ having a separate outer layer 130, the damping member 102 can be molded, formed, or otherwise extruded to enclose a cavity. For example, as shown in FIGS. 3B-3D, the damping member 102′ can include an inner layer 116, an outer layer 118, and a cavity 119 therebetween. The cavity 119 can be at least partially filled with a fluid, such as a gas, liquid, or gel.

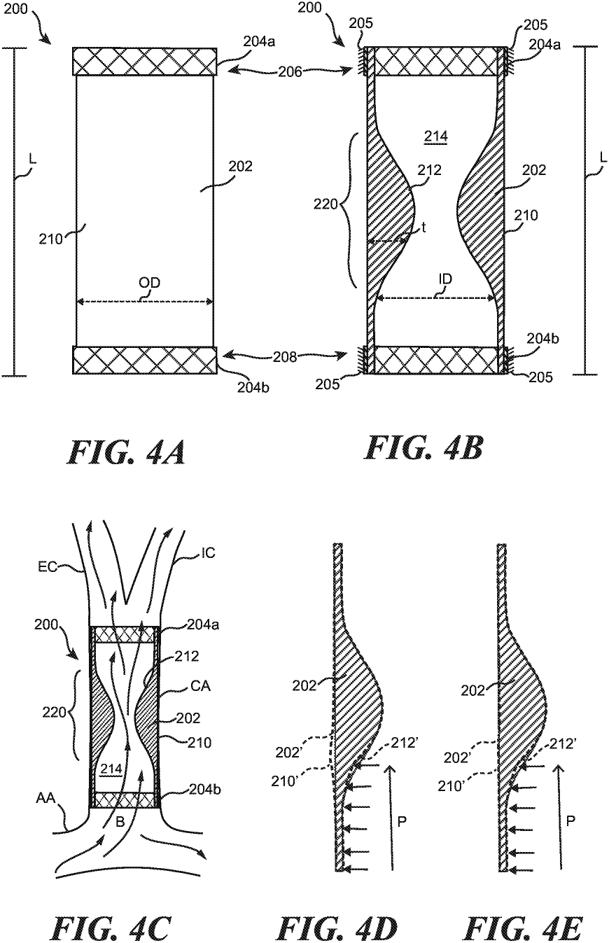

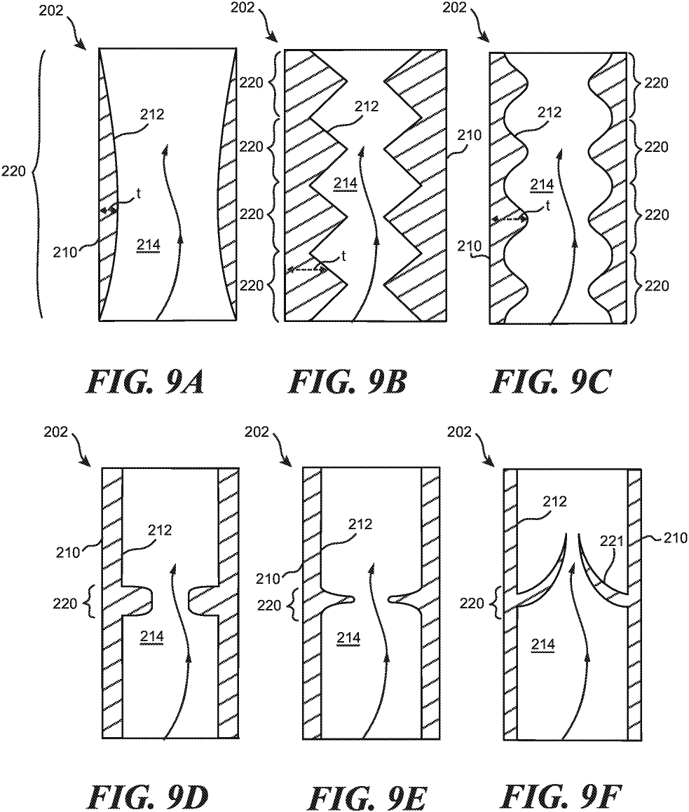

FIGS. 4A and 4B are a front view and a front cross-sectional view, respectively, of another embodiment of a damping device 200 configured in accordance with the present technology shown in an expanded or deployed state. FIG. 4C is a front cross-sectional view of the damping device 200 in a deployed state positioned in a carotid artery (e.g., the left or right carotid artery). Referring to FIGS. 4A-4C together, the damping device 200 includes a flexible, viscoelastic damping member 202 (e.g., a cushioning member) and anchoring members 204 (identified individually as first and second anchoring members 204a-204b, respectively). As shown in FIGS. 4B and 4C, the damping member 202 includes a generally tubular sidewall having a cylindrical outer surface 210 and an inner surface 212 that defines a lumen 214 configured to receive blood flow therethrough. The outer surface 210 is separated from the inner surface 212 by a distance t (FIG. 4B). The damping member 202 has a length L, a first end portion 206, and a second end portion 208 opposite the first end portion 206 along its length L, and a damping region 220 between the first end portion 206 and the second end portion 208. Along the damping region 220, the distance t between the outer and inner surfaces 210 and 212 increases then decreases in a radial direction when the damping member 202 is in a deployed, relaxed state. On average, the distance t between the outer and inner surfaces 210 and 212 of the damping member 202 is greater at the damping region 220 than at either of the first or second end portions 206, 208. As a result, the inner diameter ID of the damping member 202 varies along its length L relative to the outer diameter OD of the damping member 202. For example, the outer surface 210 can be generally cylindrical in an unbiased state, and the inner surface 212 can have an undulating or hourglass shape. As described in greater detail below with respect to FIGS. 9A-9F, the damping member 202 can have other suitable shapes, sizes, and/or configurations.

The damping member 202 shown in FIGS. 4A-4C is a solid piece of material that is molded, extruded, or otherwise formed into the desired shape. The damping member 202 can be made of a biocompatible, compliant, viscoelastic material that is configured to deform in response to local fluid pressure in the artery. As the damping member 202 deforms, the damping member 202 absorbs a portion of the pulse pressure. The damping member 202, for example, can be made of a biocompatible synthetic elastomer, such as silicone rubber (VMQ), Tufel I and Tufel III elastomers (GE Advanced Materials, Pittsfield, MA), Sorbothane ® (Sorbothane, Incorporated, Kent, OH), and others. The damping member 202 can be flexible and elastic such that the inner diameter ID of the damping member 202 at the damping region 220 increases as a systolic pressure wave P (FIG. 4D) propagates through the damping region 220. For example, as shown schematically in the isolated, cross-sectional view of a portion of a damping member 202 before and during deformation (damping member 202′, shown in dashed lines) in FIG. 4D, the systolic pressure wave P may push the inner surface 212′ radially outwardly, thus forcing a portion of the outer surface 210′ to also deform radially outwardly. Additionally, the damping member 202 can also optionally be compressible such that the distance t between the inner and outer surfaces 210 and 212 decreases to further open the inner diameter ID of the damping region 220 as the systolic pressure wave P engages the damping region 220. For example, as shown schematically in the isolated, cross-sectional view of a portion of a damping member 202 before and during deformation (damping member 202′, shown in dashed lines) in FIG. 4E, the systolic pressure wave P may push the inner surface 212′ radially outwardly while the contour of the outer surface 210′ remains generally unaffected.

In the embodiment shown in FIGS. 4A-4C, the anchoring members 204a-204b individually comprise a generally cylindrical structure configured to expand from a low-profile state to a deployed state in apposition with the blood vessel wall. Each of the anchoring members 204a-b can be a stent formed from a laser cut metal, such as a superelastic material (e.g., Nitinol) or stainless steel. All or a portion of each of the anchoring members can include a radiopaque coating to improve visualization of the device during delivery, and/or the anchoring members may include one or more radiopaque markers. In other embodiments, the individual anchoring members 204a-204b can comprise a mesh or woven (e.g., a braid) construction in addition to or in place of a laser cut stent. For example, the individual anchoring members 204a-204b can include a tube or braided mesh formed from a plurality of flexible wires or filaments arranged in a diamond pattern or other configuration. In some embodiments, all or a portion of one or both of the anchoring members 204a-204b can be covered by a graft material (such as Dacron) to promote sealing with the vessel wall.

In the embodiment shown in FIGS. 4A-4B, the anchoring members 204a-204b are positioned around the damping member 202 at the first and second end portions 206, 208, respectively. As such, in this embodiment, the outer diameter OD (FIG. 4A) of the damping member 202 is less than the inner diameter of the anchoring members 204a-204b. Also in the embodiment shown in FIGS. 4A-4B, the anchoring members 204a-204b are positioned around the damping member 202 only at the first and second end portions 206, 208, respectively. As such, in several embodiments of the present technology, the damping region 220 of the damping member 220 is not surrounded by a stent-like structure or braided material. In other embodiments, the anchoring members 204a-204b and damping member 202 may have other suitable configurations. For example, the anchoring members 204a-204b may be positioned at other locations along the length L of the damping member 202, though not along the full length of the damping member 202. Also, in some embodiments, all or a portion of one or both anchoring members 204a-204b may be positioned radially outwardly of all or a portion of the damping member 202. Although the damping device 200 shown in FIGS. 4A-4B includes two anchoring members 204a-204b, in other embodiments the damping device 200 can have more or fewer anchoring members (e.g., one anchoring member, three anchoring members, four anchoring members, etc.).

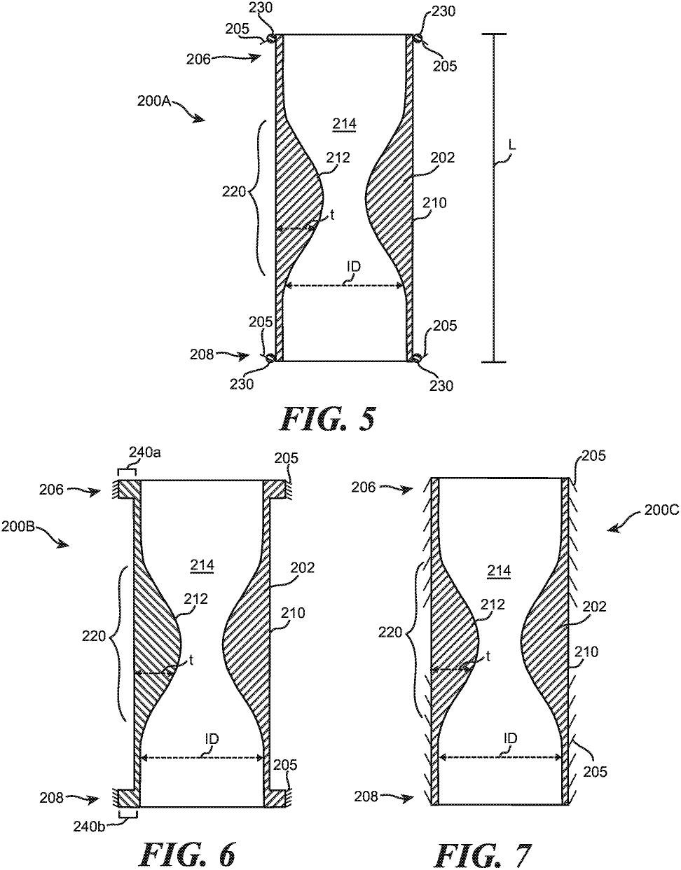

In some embodiments, one or both of the anchoring members 204a-204b can optionally include one or more fixation elements 205 (FIG. 4B) configured to engage the blood vessel wall. The fixation elements 205 can include, for example, one or more hooks or barbs that, in the deployed state, extend outwardly away from the corresponding frames of the anchoring member 204a-204b to penetrate the vessel wall at the treatment site. In these and other embodiments, one or more of the fixation elements can be atraumatic. Additionally, referring to the damping device 200A shown in FIG. 5, in certain embodiments the damping device 200 may not include a stent-type or braid-type anchoring member, but rather the frame of the anchoring members 204 can be one or more expandable rings 230. For example, in some embodiments the damping device 200 can include two rings 230, each attached to a respective end portion 206 and 208, and the plurality of fixation elements 205 can extend outwardly from the rings 230. In still other embodiments, such as the damping device 200B shown in FIG. 6, the anchoring members 204 can be integral portions of the end portions 206, 208, such as thick wall portions 240a-b of the damping member 202 that extend radially outward from the outer wall of the damping region 220, instead of separate metal or polymeric components. In this embodiment, the fixation elements 205 can extend outwardly from integral anchoring members 240a-b at the first and second end portions 206, 208 of the damping member 202. When the damping device 200 is in a deployed state, the fixation elements 205 extend outwardly away from the outer surface of the damping member 202 to engage vessel wall tissue. In yet other embodiments, the fixation elements 205 can extend outwardly from the outer surface 210 of the damping member 202, as shown in the damping device 200C of FIG. 7.

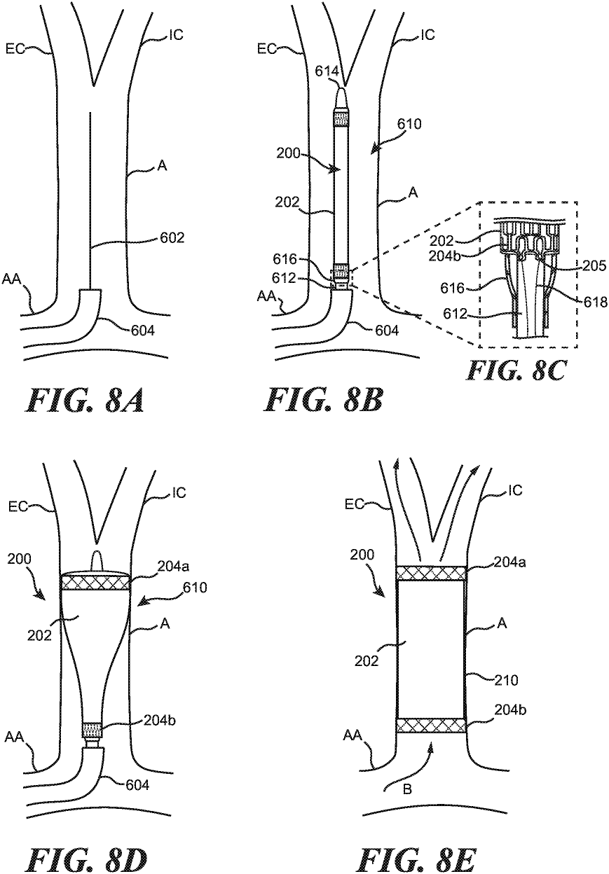

FIGS. 8A-8E illustrate a method for positioning a damping device of the present disclosure at a treatment location within an artery A (such as the left and/or right common carotid artery CA). Although FIGS. 8B-8E depict the damping device 200 shown in FIGS. 4A and 4B, the methods and systems described with respect to FIGS. 8A-8E can be utilized for any of the damping devices 100, 100′, 200, 200A, 200B, and 200C described with respect to FIGS. 2A-7 and FIGS. 9A-9F.

As shown in FIG. 8A, a guidewire 602 may first be advanced intravascularly to the treatment site from an access site, such as a femoral or a radial artery. A guide catheter 604 may then be advanced along the guidewire 602 until at least a distal portion of the guide catheter 604 is positioned at the treatment site. In these and other embodiments, a rapid-exchange technique may be utilized. In some embodiments, the guide catheter 604 may have a pre-shaped or steerable distal end portion to direct the guide catheter 604 through one or more bends in the vasculature. For example, the guide catheter 604 shown in FIGS. 8A-8E has a curved distal end portion configured to navigate through the ascending aorta AA and preferentially bend or flex at the left and/or right common carotid artery A to direct the guide catheter 604 into the artery A.

Image guidance, e.g., computed tomography (CT), fluoroscopy, angiography, intravascular ultrasound (IVUS), optical coherence tomography (OCT), or another suitable guidance modality, or combinations thereof, may be used to aid the clinician’s positioning and manipulation of the damping device 200. For example, a fluoroscopy system (e.g., including a flat-panel detector, x-ray, or c-arm) can be rotated to accurately visualize and identify the target treatment site. In other embodiments, the treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering the damping device 200. Further, in some embodiments, image guidance components (e.g., IVUS, OCT) may be integrated with the delivery catheter and/or run in parallel with the delivery catheter to provide image guidance during positioning of the damping device 200.

Once the guide catheter 604 is positioned at the treatment site, the guidewire 602 may be withdrawn. As shown in FIGS. 8B and 8C, a delivery assembly 610 carrying the damping device 200 may then be advanced distally through the guide catheter 604 to the treatment site. In some embodiments, the delivery assembly 610 includes an elongated shaft 612 having an atraumatic distal tip 614 (FIG. 8B) and an expandable member 616 (e.g., an inflatable balloon, an expandable cage, etc.) positioned around a distal portion of the elongated shaft 612. The damping device 200 can be positioned around the expandable member 616. As shown in FIG. 8D, expansion or inflation of the expandable member 616 forces at least a portion of the damping device 200 radially outwardly into contact with the arterial wall. In some embodiments, the delivery assembly 610 can include a distal expandable member for deploying a distal portion of the damping device 200, and a proximal expandable member for deploying a proximal portion of the damping device 200. In other embodiments, the entire length of the damping device 200 may be expanded at the same time by deploying one or more expandable members.

In some procedures the clinician may want to stretch or elongate the damping device 200 before deploying the proximal second anchoring member 204b against the arterial wall. To address this need, the delivery assembly 610 and/or damping device 200 can optionally include a tensioning mechanism for pulling or providing a tensile stress on the second anchoring member 204b, thereby increasing the length of the damping member 202 and/or a distance between the first and second anchoring members 204a, 204b. For example, as shown in FIG. 8C, the second anchoring member 204b can include one or more coupling portions 205 (e.g., one or more eyelets extending proximally from the anchoring frame) and one or more coupling members 618 (e.g., a suture, a thread, a filament, a tether, etc.) extending between the second anchoring member 204b and a proximal portion (not shown) of the delivery assembly 610 (e.g., a handle). The coupling members 618 are configured to releasably engage the coupling portions 205 to mechanically couple the second anchoring member 204b to a proximal portion of the delivery assembly 610. A clinician can apply a tensile force to the coupling member 618 to elongate the damping device 200 and/or damping member 202 and adjust the longitudinal position of the second anchoring member 204b. Once the second anchoring member 204b is positioned at a desired longitudinal position relative to the first anchoring member 204a and/or the local anatomy, the second anchoring member 204b can be expanded into contact with the arterial wall (e.g., via deployment of one or more expandable members). Before, during, and/or after expansion of the second anchoring member 204b, the coupling member(s) 618 may be disengaged from the second anchoring member 204b. For example, in some embodiments, the operator can force the coupling members 618 to break along their lengths by applying a tensile force that is less than a force that would be required to dislodge one or both of the first and second anchoring members 204a, 204b. Once disengaged from the second anchoring member 204b and/or the damping device 200, the coupling member(s) 618 can then be withdrawn from the treatment site through the guide catheter 604.

In other embodiments, other tensioning mechanisms may be utilized. For example, in some embodiments, the damping device 200 includes a releasable clasp, ring, or hook which is selectively releasable by the operator. The clasp, ring or hook may be any type that permits securement of the thread to the second anchoring member 204b, and which can be selectively opened or released to disengage the thread from the second anchoring member 204b. The releasing can be controlled by the clinician from an extracorporeal location. Although the tensioning mechanism is described herein with respect to the second anchoring member 204b, it will be appreciated that other portions of the damping device 200 and/or the delivery assembly 610 (such as the first anchoring member 204a) can be coupled to a tensioning mechanism.

In certain embodiments, the damping member 202 and/or individual anchoring members 204a, 204b may be self-expanding. For example, the delivery assembly 610 can include a delivery sheath (not shown) that surrounds and radially constrains the damping device 200 during delivery to the treatment site. Upon reaching the treatment site, the delivery sheath may be at least partially withdrawn or retracted to allow the damping member 202 and/or the individual anchoring members 204a, 204b to expand. In some embodiments, expansion of the anchoring members 204 may drive expansion of the damping member 202. For example, the anchoring members 204 may be fixedly attached to the damping member 202, and expansion of one or both anchoring 204 pulls or pushes (depending on the relative positioning of the damping member 202 and anchoring members 204) the damping member 202 radially outwardly.

As best shown in FIG. 8C, once the damping device 200 is positioned at the treatment site (e.g., in a left or right common carotid artery), oxygenated blood ejected from the left ventricle flows through the lumen 214 of the damping member 202. As the blood contacts the damping region 220 of the damping member 202, the damping region 220 deforms to absorb a portion of the pulsatile energy of the blood, which reduces a magnitude of a pulse pressure transmitted to the portions of the artery distal to the damping device 200 (such as the more-sensitive cerebral arteries). The damping region 202 acts a pressure limiter that distributes the pressure of the systolic phase of the cardiac cycle more evenly downstream from the damping device 200 without unduly compromising the volume of blood flow through the damping device 200. Accordingly, the damping device 200 reduces the pulsatile stress on downstream portions of the arterial network to prevent or at least partially reduce the manifestations of vascular dementia and/or age-related dementia.

In some procedures, it may be beneficial to deliver multiple damping devices 200 to multiple arterial locations. For example, after deploying a first damping device 200 at a first arterial location (e.g., the left or right common carotid artery, an internal or external carotid artery, the ascending aorta, etc.), the clinician may then position and deploy a second damping device 200 at a second arterial location different than the first arterial location (e.g., the left or right common carotid artery, an internal or external carotid artery, the ascending aorta etc.). In a particular application, a first damping device is deployed in the left common carotid artery and the second damping device is deployed in the right common carotid artery. In other embodiments, two or more damping devices 200 may be delivered simultaneously.

In some embodiments, an additional stent of larger diameter may be placed within the vessel prior to deployment of the damping device 200 to expand the diameter of the vessel in preparation for the device. Subsequently, the damping device 200 can be deployed within the larger stent. This may assist to reduce impact on the residual diameter of the vessel, and thereby reduce impact on blood flow rate.

FIGS. 9A-9F are schematic cross-sectional views of several embodiments of damping members in accordance with the present technology. Like reference numbers refer to similar or identical components in FIGS. 2A-9F. In the embodiment shown in FIG. 9A, the inner surface 212 of the damping member 202 is curved along its entire length. The distance between the outer surface 210 and the inner surface 212 gradually increases then decreases in a distal direction. As such, the damping region 220 extends the entire length of the damping member 202. FIGS. 9B and 9C illustrate embodiments of the damping member 202 in which the inner surface 212 has a series of damping regions 220 defined by undulations in the inner surface 212. In these embodiments, the distance t increases, then decreases, then increases, then decreases, etc. in a distal direction. In FIG. 9B, the damping regions 220 are generally linear, while in FIG. 9C, the damping regions 220 are generally curved. FIGS. 9D-9E illustrate embodiments of damping members 202 having damping regions 220 comprising an annular ring projecting radially inwardly into the lumen 214. One or more portions of the annular ring may flex in a longitudinal direction in response to blood flow. As shown in FIG. 9F, in some embodiments the damping member 220 can comprise two or more opposing leaflets 221.

### II. Selected Resection Embodiments of Damping Devices

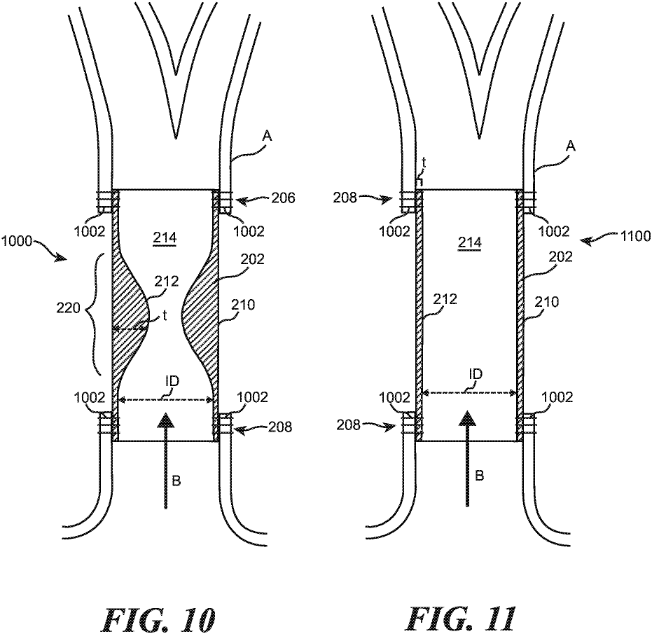

FIGS. 10 and 11 are schematic cross-sectional views of several embodiments of damping devices in accordance with the present technology. Like reference numbers refer to similar or identical components in FIGS. 2A-15. FIG. 10, for example, shows a damping device 1000 comprising only the damping member 202. A portion of the arterial wall A may be resected, and the damping member 202 may be coupled to the open ends of the resected artery (e.g., via sutures 1002) such that the damping member 202 spans the resected portion of the artery A. In some embodiments, the damping member 202 may have a generally cylindrical shape with a constant wall thickness, as shown in FIG. 11. In such embodiments, an inner diameter ID of the damping member 202 may be generally constant along the length of the damping member 202. In operation, the damping devices 1000 and 1100 shown in FIGS. 10 and 11 are highly flexible, elastic members that expand radially outward as the systolic pressure wave passes through the damping devices 1000 and 1100. Since the resected portions of the arterial wall A cannot limit the expansion of the damping devices 1000 and 1100, these devices can expand more than the native arterial wall A to absorb more energy from the blood flow.

### III. Selected Additional Embodiments of Damping Devices

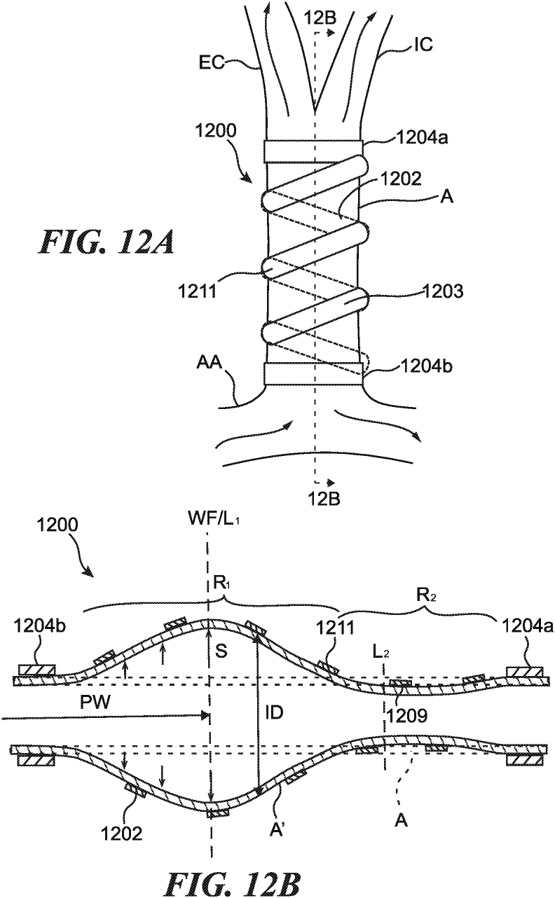

FIGS. 12A-19B illustrate additional embodiments of damping devices configured in accordance with the present technology. For example, FIG. 12A shows a damping device 1200 comprising a damping member 1202 coupled to anchoring members 1204a and 1204b at its proximal and distal end portions. The damping member 1202 comprises a strand 1203 having a pre-set helical configuration such that, in a deployed state, the strand 1203 forms a generally tubular structure defining a lumen extending therethrough. The tubular structure has an inner surface 1209 (FIG. 12B) and an outer surface 1211. The strand 1203 may be formed of any suitable biocompatible material such as one or more elastic polymers that are configured to stretch in response to the radially outward forces exerted by the pulse wave on the helical strand. In some embodiments, the strand 1203 may additionally or alternatively include one or more metals such as stainless steel and/or a superelastic and/or shape memory alloy, such as Nitinol. In a particular embodiment, the damping member 1202 may be fabricated from a recombinant human protein such as tropo-elastin or elastin.

The anchoring members 1204a and 1204b can be generally similar to the anchoring members 104a and 104b described with respect to FIGS. 2A-2C. In some embodiments, the damping device 1200 includes more or fewer than two anchoring members 1204 (one anchoring member, three anchoring members, etc.). In a particular embodiment, the damping device 1200 does not include anchoring members 1204.

In the deployed state, the damping member 1202 is configured to be wrapped along the circumference of an artery that supplies blood to the brain. For example, in the embodiment shown in FIG. 12A, the damping member 1202 is configured to be positioned around the exterior of the artery A such that the inner surface 1209 of the damping member 1202 contacts an outer surface of the artery A (see FIG. 12B). In other embodiments (not shown), the damping member 1202 is configured to be positioned around the lumen of the artery such that the outer surface 1211 of the damping member 1202 contacts an inner surface of the arterial wall.

FIG. 12B is a cross-sectional side view of the damping device 1200 during transmission of a pulse wave PW through the portion of the artery A surrounded by the damping device 1200. In FIG. 12B, the dashed lines A represent the artery during diastole, or when the artery is relaxed. The solid line A′ represents the artery in response to a pulse wave PW traveling through the artery during systole. As shown in FIG. 12B, as the wave front WF (or leading edge of the pulse wave PW) travels through the artery, the wave front dilates the artery A at an axial location Li corresponding to the wave front WF. The wave front WF pushes the arterial wall radially outwardly against the coil, thereby radially expanding the portion Ri of the coil axially aligned with the wave front WF. For example, in those embodiments where the strand 1203 is made of a stretchable material, such as an elastic polymer, the coil stretches along the portion Ri to expand and accommodate the pulse wave, thereby absorbing some of the energy transmitted with the pulse wave and reducing the stress on the arterial wall. In any of the above embodiments, the portions of the coil distal or proximal the wave-affected region are forced to contract (R₂), thereby causing the artery to narrow relative to its relaxed diameter. This narrowing of the artery creates a temporary impedance to the pulse wave which absorbs some of the energy. Once the pulse wave has passed, the arterial wall returns to its relaxed state.

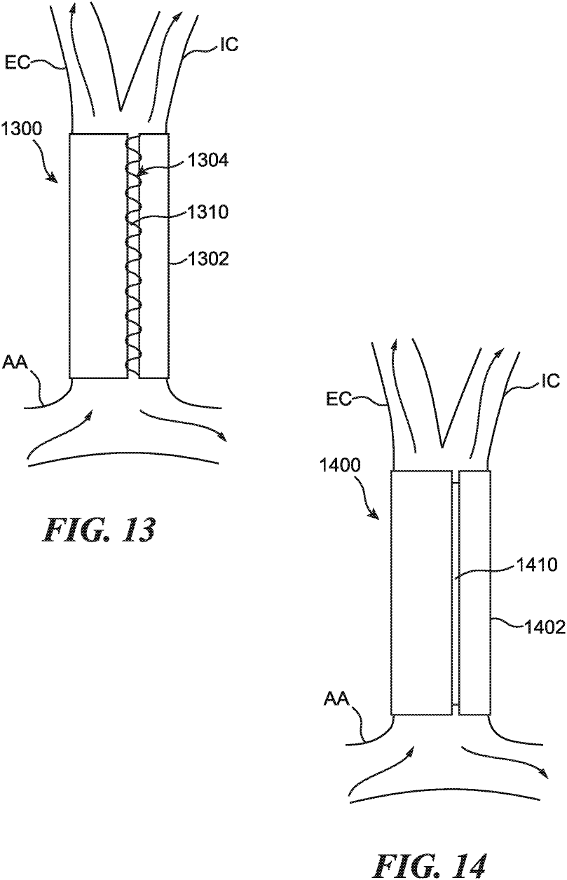

FIG. 13 illustrates another embodiment of a damping device 1300 in accordance with the present technology. As shown in FIG. 13, the damping device 1300 can include a damping member 1302 defined by an extravascular wrap. The damping member 1302 may be fabricated from a generally rectangular portion of a suitable bio-compatible and elastically deformable material which is configured to be wrapped around the blood vessel. Alternatively, the damping member 1302 may be initially provided having a cylindrical configuration including a longitudinal slit 1304 for receiving the vessel. The damping member 1302 may be fabricated from a synthetic such as an elastic polymer, a shape memory and/or superelastic material such as Nitinol (nickel titanium), a recombinant human protein such as tropo-elastin or elastin, and other suitable materials. As shown in FIG. 13, the damping member 1302 is configured to be secured around an artery (e.g., a carotid artery) between the aortic arch and the junction where the left common carotid artery divides into the internal (IC) and external (EC) carotid arteries. It will be appreciated by those skilled in the art that the damping member 1302 may alternatively or additionally be deployed around the brachiocephalic trunk (not shown) or the right common carotid artery (not shown), or any distal branch of the aforementioned arteries, or any proximal branch of the aforementioned arteries, such as the ascending aorta. Opposing edges of the damping member 1302 can be secured to each other with a coupling device such as stitching/sutures 1310, stapling, or another coupling device such that the external diameter of the artery is reduced. In some embodiments, the coupling device can be made from an elastic material so that it can stretch to accommodate the pulse wave and absorb its energy. The elastically deformable damping member 1302 is adapted to radially expand during the systole stage and radially contract during the diastole stage. The damping member 1302 is secured such that an internal diameter of the elastically deformable material is smaller than an initial, outer diameter of the artery during a systole stage, but not smaller than an outer diameter of the artery during a diastole stage.

FIG. 14 depicts another embodiment of a damping device 1400 for treating an arterial blood vessel. The device 1400 can be structurally similar to the damping device 1300 shown in FIG. 13, with the exception that the two opposing edges of the elastically deformable damping member 1402 of FIG. 14 are secured to each other using a zip-lock type coupling mechanism 1410.

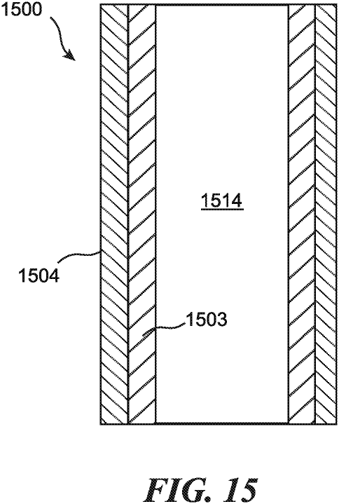

FIG. 15 shows another embodiment of a damping device 1500 configured in accordance with the present technology. The damping device 1500, includes a generally tubular anchoring member 1504 (e.g., a stent, a mesh, a braid, etc.) defining a lumen 1514 therethrough. The anchoring member may be made of a resilient, biocompatible material such as stainless steel, titanium, nitinol, etc. In some embodiments, the anchoring member 1504 is made of a shape memory and/or superelastic material. A radially outer surface of the anchoring member 1504 is configured to be positioned in apposition with an inner surface of an arterial wall. A radially inner surface of the anchoring member 1504 is lined or otherwise coated with an absorptive material 1503 (e.g., a cushioning material), such as an elastically deformable material, which is adapted to absorb shock. The lumen 1514 is configured to receive blood flow therethrough. The lumen 1514 is present when the anchoring member 1504 is radially expanded, but it may not be present in the initial, contracted configuration prior to deployment.

In some embodiments (not shown), the damping device can be a biocompatible gel which is injected around a portion of the left or right carotid artery or the brachiocephalic trunk. The gel increases the external pressure acting on the artery and thus reduces the external diameter of the artery. As blood pressure increases within the artery, the gel elastically deforms, such that the artery radially expands during the systole stage and radially contracts during the diastole stage.

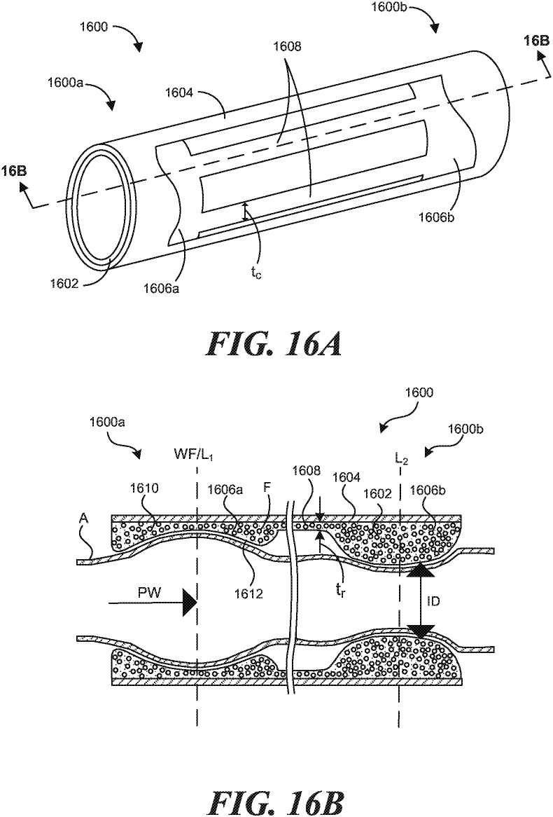

FIG. 16A is a perspective, cut-away view of a damping device 1600 in accordance with the present technology in a deployed, relaxed state. FIG. 16B is a cross-sectional view of the damping device 1600 positioned in an artery A during transmission of a pulse wave PW through the portion of the artery A surrounded by the damping device 1600. Referring to FIGS. 16A and 16B together, the damping device 1600 includes a damping member 1602 and a structural member 1604 coupled to the damping member 1602. In FIG. 16A, a middle portion of the structural member 1604 has been removed to show features of the structure of the damping member 1602. As shown in FIG. 16A, the damping device 1600 can have a generally cylindrical shape in the deployed, relaxed state. The damping device 1600 may be configured to wrap around the circumference of the artery with opposing longitudinal edges (not shown) secured to one another via sutures, staples, adhesive, and/or other suitable coupling devices. Alternatively, the damping device 1600 can have a longitudinal slit for receiving the artery therethrough. In either of the foregoing extravascular embodiments, the damping device 1600 is configured to be positioned around the circumference of the artery A so that the inner surface 1612 (FIG. 16B) is adjacent and/or in contact with the outer surface of the arterial wall. In other embodiments, the damping device 1600 can be configured to be positioned intravascularly (e.g., within the artery lumen) such that an outer surface of the damping device 1600 is adjacent and/or in contact with the inner surface of the arterial wall. In such intravascular embodiments, the inner surface 1612 of the damping member 1602 is adjacent or directly in contact with blood flowing through the artery A.

The structural member 1604 can be a generally cylindrical structure configured to expand from a low-profile state to a deployed state. The structural member 1604 is configured to provide structural support to secure the damping device 1600 to a selected region of the artery. In some embodiments, the structural member 1604 can be a stent formed from a laser cut metal, such as a superelastic and/or shape memory material (e.g., Nitinol) or stainless steel. All or a portion of the structural member 1604 can include a radiopaque coating to improve visualization of the device 1600 during delivery, and/or the structural member 1604 may include one or more radiopaque markers. In other embodiments, the structural member 1604 may comprise a mesh or woven (e.g., a braid) construction in addition to or in place of a laser cut stent. For example, the structural member 1604 can include a tube or braided mesh formed from a plurality of flexible wires or filaments arranged in a diamond pattern or other configuration. In some embodiments, all or a portion of the structural member 1604 can be covered by a graft material (such as Dacron) to promote sealing with the vessel wall. Additionally, all or a portion of the structural member 1604 can include one or more biomaterials.

In the embodiment shown in FIGS. 16A and 16B, the structural member 1604 is positioned radially outwardly of the damping member 1602 and extends along the entire length of the damping member 1602 (though a middle portion of the structural member 1604 is cut-away in FIG. 16A for illustrative purposes only). In other embodiments, the structural member 1604 and the damping member 1602 may have other suitable configurations. For example, the damping device 1600 can include more than one structural member 1604 (e.g., two structural members, three structural members, etc.). Additionally, in some embodiments the structural member(s) 1604 may extend along only a portion of the damping member 1602 such that a portion of the length of the damping member 1602 is not surrounded and/or axially aligned with any portion of the structural member 1604. Also, in some embodiments, all or a portion of the damping member 1602 may be positioned radially outwardly of all or a portion of the structural member 1604.

In the embodiment shown in FIGS. 16A and 16B, the damping member 1602 includes a proximal damping element 1606a and a distal damping element 1606b. The damping member 1602 may further include optional channels 1608 extending between the proximal and distal damping elements 1606a, 1606b. The channels 1608, for example, can extend in a longitudinal direction along the damping device 1600 and fluidly couple the proximal damping element 1606a to the distal damping element 1606b. The damping member 1602 may further include an abating substance 1610 configured to deform in response to fluid stress (such as blood flow), thereby absorbing at least a portion of the stress. For example, as best shown in FIG. 16B, in one embodiment the abating substance 1610 includes a plurality of fluid particles F (only one fluid particle labeled) contained in the proximal damping element 1606a, distal damping element 1606b, and channel(s) 1608. As used herein, the term “fluid” refers to liquids and/or gases, and “fluid particles” refers to liquid particles and/or gas particles. In some embodiments, the damping member 1602 is a gel, and the plurality of fluid particles F are dispersed within a network of solid particles. In other embodiments, the damping member 1602 may include only fluid particles F (e.g., only gas particles, only liquid particles, or only gas and liquid particles) contained within a flexible and/or elastic membrane that defines the proximal damping member 1606a, the distal damping member 1606b, and the channel(s) 1608. The viscosity and/or composition of the abating substance 1610 may be the same or may vary along the length and/or circumference of the damping member 1602.

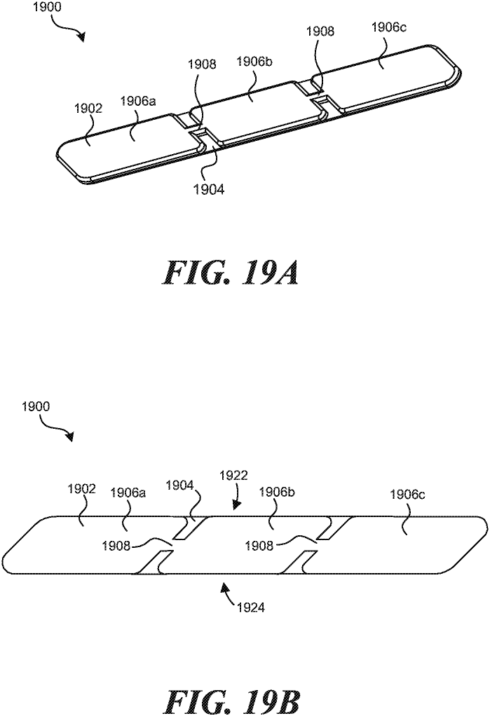

In the embodiment shown in FIGS. 16A and 16B, the channels 1608 have a resting radial thickness tᵣ and circumferential thickness t_{c} (FIG. 16A) that is less than the resting radial thickness tᵣ and circumferential thickness t_{c}, respectively, of the proximal and distal damping elements 1606a, 1606b. As best shown in FIG. 16A, in some embodiments the proximal and distal damping elements 1606a and 1606b may extend around the full circumference of the damping device 1600 and the channels 1608 may extend around only a portion of the circumference of the damping device 1600. In other embodiments, the channels 1608 can have a resting radial thickness tᵣ that is generally the same as that of the proximal and distal damping elements 1606a, 1606b (see damping elements 1906a-c and channels 1908 in FIGS. 19A and 19B) and/or a resting circumferential thickness t_{c} that is generally the same as that of the proximal and distal damping elements 1606a, 1606b.

Referring to FIG. 16B, when a pulse wave PW traveling through the artery A applies a stress at a first axial location Li along the length of the damping member 1602 (e.g., at wave front WF), at least a portion of the fluid particles move away from the first axial location Li to a second axial location L₂ along the length of the damping member 1602. As such, at least a portion of the fluid particles are redistributed along the length of the damping member 1602 such that the inner diameter ID of the damping member 1602 increases at the first axial location Li while the inner diameter ID decreases at another axial location (e.g., L₂). For example, as the wave front WF passes through the proximal portion 1600a of the device 1600, the portion of the artery A aligned with the wave front WF dilates, thereby applying a stress to the proximal damping element 1606a and forcing at least some of the fluid particles in the proximal damping element 1606a to move distally within the damping member 1602. At least some of the displaced fluid particles are forced through the channel(s) 1608 and into the distal damping element 1606b, thereby increasing the volume of the distal damping element 1606b and decreasing the inner diameter ID of the damping device 1600 at the distal portion 1600b. The decreased inner diameter ID of the damping device 1600 provides an impedance to the blood flow that absorbs at least a portion of the energy in the pulse wave when the blood flow reaches the distal damping member 1606b. As the wave front WF then passes through the distal portion 1600b of the device 1600, the portion of the artery A aligned with the wave front WF dilates, thereby applying a stress to the distal damping element 1606b and forcing at least some of the fluid particles currently in the distal damping element 1606b to move proximally within the damping member 1602. At least some of the displaced fluid particles are forced through the channel(s) 1608 and into the proximal damping element 1606a, thereby increasing the volume of the proximal damping element 1606a and decreasing the inner diameter ID of the device 1600 at the proximal portion 1600a. Movement of the fluid particles and/or deformation of the damping member 1602 in response to the pulse wave absorbs at least a portion of the energy carried by the pulse wave, thereby reducing the stress on the arterial wall distal to the device.

When the damping member 1602 deforms in response to the pulse wave, the shape of the structural member 1604 may remain generally unchanged, thereby providing the support to facilitate redistribution of the fluid particles within and along the damping member 1602. In other embodiments, the structural member 1604 may also deform in response to the local fluid stress.

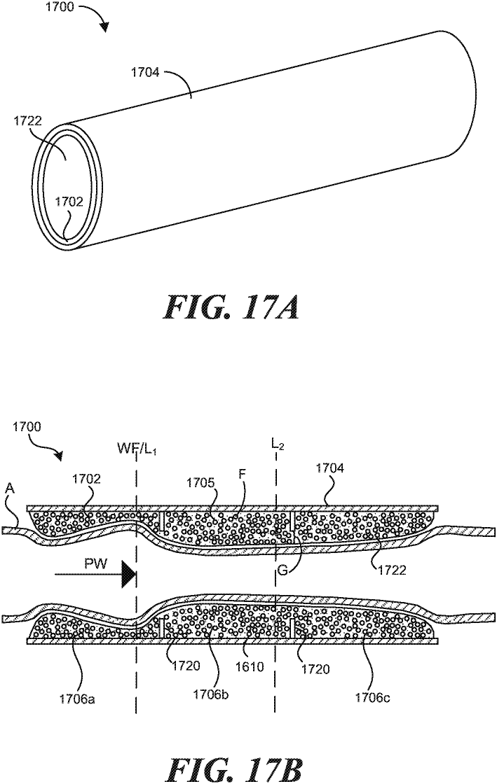

FIG. 17A is a perspective view of another embodiment of a damping device 1700 in accordance with the present technology. FIG. 17B is a cross-sectional view of the damping device 1700 positioned in an artery A during transmission of a pulse wave PW through the portion of the artery A surrounded by the damping device 1700. The damping device 1700 can include a structural member 1704 and a damping member 1702. The structural member 1704 can be generally similar to the structural member 1604 shown in FIGS. 16A and 16B. The damping member 1702 is defined by a single chamber 1705 including an abating substance 1610 and a plurality of baffles 1720 that separate the chamber 1705 into three fluidically-coupled compartments 1706a, 1706b, and 1706c. The baffles 1720 extend only a portion of the radial thickness of the damping member 1702, thereby leaving a gap G between the end of the baffles 1720 and an inner wall 1722 of the damping member 1702. In other embodiments, the damping device 1700 can include more or fewer compartments (e.g., a single, tubular compartment (no baffles), two compartments, four compartments, etc.). Moreover, the baffles 1720 may extend around all or a portion of the circumference of the damping member 1702.

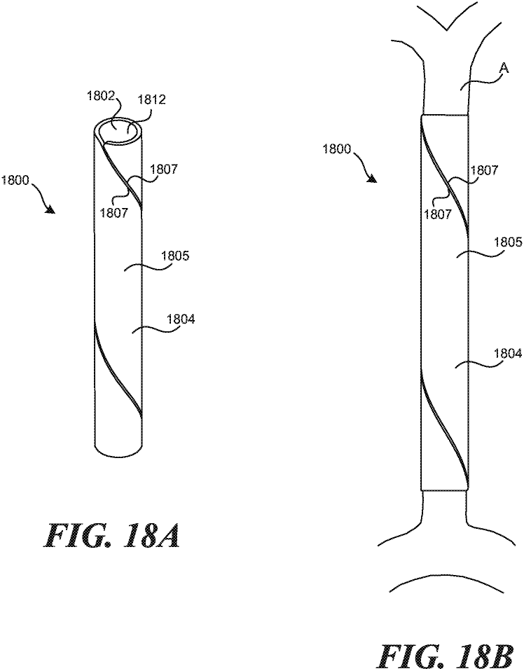

FIG. 18A is a perspective view of another embodiment of a damping device 1800 in accordance with the present technology, and FIG. 18B is a front view of the damping device 1800, shown in a deployed state positioned around an artery A. Referring to FIGS. 18A-18B together, the damping device 1800, in a deployed, relaxed state, includes a generally tubular sidewall 1805 that defines a lumen. The damping device 1800 can be formed of a generally parallelogram-shaped element that is wrapped around a mandrel in a helical configuration and heat set. In other embodiments, the damping device 1800 can have other suitable shapes and configurations in the unfurled, non-deployed state. As shown in FIG. 18B, in the deployed state, the damping device 1800 is configured to be wrapped helically along or around the circumference of an artery supplying blood to the brain. Opposing longitudinal edges 1807 of the damping device 1800 come together in the deployed state to form a helical path along the longitudinal axis of the artery A. The damping device 1800 can include any of the coupling devices described with respect to FIGS. 13-15 to secure all or a portion of the opposing longitudinal edges to one another.

As best shown in FIG. 18A, the sidewall 1805 of the damping device 1800 includes a structural member 1804 and a damping member 1802. The structural member 1804 can be generally similar to the structural member 1604 shown in FIGS. 16A and 16B, except the structural member 1804 of FIGS. 18A and 18B has a helical configuration in the deployed state. The damping member 1802 can be generally similar to any of the damping members described herein, especially those described with respect to FIGS. 13-17B and 19A and 19B. In the embodiment shown in FIGS. 18A and 18B, the damping member 1802 is positioned radially inwardly of the structural member 1804 when the damping device 1800 is in the deployed state. In other embodiments, the damping member 1802 may be positioned radially outwardly of the structural member 1804 when the damping device 1800 is in the deployed state.

The damping device 1800 may be configured to wrap around the circumference of the artery A so that the inner surface 1812 (FIG. 18A) is adjacent and/or in contact with the outer surface of the arterial wall. In other embodiments, the damping device 1800 can be configured to be positioned intravascularly (e.g., within the artery lumen) such that an outer surface of the damping device 1800 is adjacent and/or in contact with the inner surface of the arterial wall. In such intravascular embodiments, the inner surface 1812 of the damping member 1802 is adjacent or directly in contact with blood flowing through the artery A.

FIGS. 19A and 19B are perspective and top views, respectively, of a damping device 1900 that can define one embodiment of the damping device 1800 shown in FIGS. 18A and 18B. In FIGS. 19A and 19B, the damping device 1900 is shown in an unfurled, non-deployed state. The damping device 1900 includes a damping member 1902 having a plurality of chambers 1906a, 1906b, 1906c spaced apart along a longitudinal dimension of the damping device 1900 in the unfurled state. The chambers 1906a, 1906b, 1906c may be fluidly coupled by channels 1908 extending between adjacent chambers. The damping device 1900 can thus operate in a manner similar to the damping device 1600 where an abating substance (not shown in FIGS. 19A and 19B) in the chambers 1906a-c moves through the channels 1908 to inflate/deflate individual chambers in response to a pressure wave traveling through the blood vessel. The displacement of the abating substance within the chambers 1906a-c attenuates the energy of the pulse wave to reduce the impact of the pulse wave distally of the damping device 1900.

### IV. Selected Biologic Therapeutic Agents for Treating Neurological Conditions

In addition to providing the implantable damping device, the present technology includes providing biologic therapeutic agents for treating neurological disorders. One of ordinary skill in the art will understand that the biologic therapeutic agents discussed herein are illustrative of the type of biologic therapeutic agents in the present technology, and that the present technology is not limited to the biologic therapeutic agents explicitly discussed herein. For example, biologic therapeutic agents not explicitly described herein but that are within the classes of biologic therapeutic agents provided herein and/or treat the neurological conditions discussed herein are included in the present technology.

Biologic therapeutic agents for treating neurological conditions, such as neurocognitive and/or neurodegenerative disorders, include biologic therapeutic agents approved for use in human subjects by the Food and Drug Administration of the United States of America (“FDA”), biologic therapeutic agents currently in clinical trials to investigate their use in human subjects such as clinical trials governed by the FDA or other similar organizations in other countries, pre-clinical biologic therapeutic agents, and any other biologic therapeutic agent for treating a neurological condition, or intended to treat a neurological condition such as investigative biologic therapeutic agents, biologic therapeutic agents that are undergoing development or otherwise being considered for development, and biologic therapeutic agents that have been identified as potentially useful for treating or intending to treat the neurological condition. Examples of neurological conditions, such neurocognitive, neurodegenerative, or other neurological disorders include, but are not limited to, Alzheimer’s disease, mild Alzheimer’s disease, prodromal Alzheimer’s disease, mild cognitive impairment, cerebral amyloid angiopathy, frontotemporal dementia, vascular dementia, age-related dementia, amyloidosis, Lewy body disease, Parkinson’s disease, Huntington’s disease, multiple sclerosis, amyotrophic lateral sclerosis, Friedreich’s ataxia, and traumatic brain injury. In some embodiments, these biologic therapeutic agents represent more than one class of biologic therapeutic agents, more than one mechanism of action, more than one therapeutic target, and more than one therapeutic purposes.

The biologic therapeutic agents discussed herein have different therapeutic purposes, such as disease modifying biologic therapeutic agents, symptomatic cognitive enhancers, and/or symptomatic agents addressing neuropsychiatric and behavioral changes. Disease modifying biologic therapeutic agents, for example, alter the pathophysiology of the neurological condition. Symptomatic biologic therapeutic agents, for example, mitigate and/or alleviate symptoms associated with the neurological condition. In some embodiments, a biologic therapeutic agent is a disease modifying therapy and a symptomatic therapy. In some embodiments, a biologic therapeutic agent may include more than one biologic therapeutic agent.

In some embodiments, biologic therapeutic agents of the present technology are members of general classes of biologic therapeutic agents which include, but are not limited to, DNA-based biologic therapeutic agents, RNA-based biologic therapeutic agents, stem-cell biologic therapeutic agents, and natural biologic therapeutic agents. Each of these general classes of biologic therapeutic agents include subclasses having different mechanisms of action and therapeutic effects.

The biologic therapeutic agents discussed herein have different therapeutic targets, activities, and effects. For example, biologic therapeutic agents of the present technology include anti-amyloid biologic therapeutic agents, anti-tau biologic therapeutic agents, anti-inflammatory biologic therapeutic agents, neuroprotective biologic therapeutic agents, neurotransmitter-based biologic therapeutic agents, metabolic biologic therapeutic agents, antiviral biologic therapeutic agents, cell-based biologic therapeutic agents, gene-based biologic therapeutic agents, microbiome-based biologic therapeutic agents, and regenerative biologic therapeutic agents. Other types of biologic therapeutic agents include biologic neurotransmitter modulating agents, biologic membrane contact site modifier, and biologic neurotrophic factors. In some embodiments, biologic therapeutic agents have more than one therapeutic effect. For example, biologic therapeutic agents have one, two, three, four, five, or more different therapeutic effects. For example, in some embodiments, a biologic therapeutic agent is a neurotrophic therapy and an APOE therapy, or in some embodiments a biologic therapeutic agent is an NGF therapy and anti-inflammatory therapy, or in some embodiments a biologic therapeutic agent is an hTERT therapy and a neuroprotective therapy, or in some embodiments a biologic therapeutic agent is a neuroprotective therapy and an antiviral therapy, or in some embodiments a biologic therapeutic agent is a stem-cell therapy or a progenitor cell therapy and an exosome therapy (e.g., a stem cell exosome therapy and/or a progenitor cell exosome therapy), or in some embodiments a biologic therapeutic agent is one or more microbiome-based therapies, or any combination of the above.

In some embodiments, biologic therapeutic agents of the present technology have different mechanisms of action. In some embodiments, a biologic therapeutic agent is selected for administration to a subject in need thereof based on its mechanism of action. For example, some biologic therapeutic agents for treating neurological conditions such as Alzheimer’s disease prevent abnormal cleavage of amyloid precursor protein in a subject’s brain. In some embodiments, biologic therapeutic agents prevent expression and/or accumulation of β-amyloid protein (Aβ) in the subject’s brain. For example, some biologic therapeutic agents prevent expression and/or accumulation of tau protein in the subject’s brain. As another example, some biologic therapeutic agents for treating neurological conditions such as Alzheimer’s disease reduce systemic inflammation, blood brain barrier inflammation, systemic inflammation, blood brain barrier inflammation, or neuroinflammation in the subject. In some embodiments, biologic therapeutic agents increase a number of stem cells and/or progenitor cells in the subject systemically and/or in the subject’s brain. In some embodiments, biologic therapeutic agents reduce or otherwise inhibit the physiological impact an APOE variant (e.g., APOE4) in the subject systemically and/or in the subject’s brain, for example, by introducing a nucleic acid encoding APOE2 and/or removing at least a portion of a nucleic acid encoding APOE4 in the subject. Molecular approaches useful for removing at least a portion of any nucleic acids in the subject (such as in one or more astrocytes, pericytes, neurons, endothelial cells, etc.), include CRISPR (e.g., CRISPR/Cas9), TALENs, zinc-finger nucleases, and the like. In some embodiments, biologic therapeutic agents increase or otherwise prevent a decrease in expression of one or more neurotrophic factors in the subject systemically and/or in the subject’s brain. In some embodiments, biologic therapies (e.g. a microbiome transplant, probiotic) reduce or otherwise inhibit the presence of an abnormal biome in the subject. In some embodiments, biologic therapies at least partially restore or otherwise provide a normal biome in the subject. In some embodiments, biologic therapeutic agents treat, prevent, or delay Alzheimer’s disease, other neurological conditions, or cognitive decline by increasing neurotransmission, decreasing inflammation (e.g., blood brain barrier inflammation, neuroinflammation, systemic inflammation), decreasing reactive oxygen species (e.g., oxidative stress), decreasing ischemia, decreasing amyloid beta, decreasing tau, and/or decreasing insulin resistance. For example, inflammation can be decreased by directly or indirectly reducing a level of one or more cytokines, chemokines, and/or inflammatory markers in the subject (e.g., circulating or in the subject’s cerebral spinal fluid (CSF)), including VCAM-1, ICAM-1, TNFα, TGF-β, IL-6, IL-8, IL-1β, IL-12, and NF-_{K}B, and/or administering a peptide-based therapeutic to the subject, such as an IL-IRa derivative (e.g., Anakinra®). Reactive oxygen species can be increased in the subject relative to before the subject has the condition or compared to a subject who does not have the condition.

Any of the biologic therapeutic agents described herein, as well as other therapeutic agents which are members of the general classes of biologic therapeutic agents described herein, are administered to the subject in need thereof at a therapeutically effective dose before, during, or after positioning the implantable damping device within a subject, such as but not limited to a subject having elevated forward compression wave intensity (FCWI) or elevated pulse pressure. Non-limiting elevated pulse pressure values include top-quartile carotid FCWI (mmHg m/s³) of at least about 10,000, a systolic blood pressure (mmHg) of at least about 125 mmHg, a pulse pressure of at least about 50 mmHg, a diastolic blood pressure of at most about 70 mmHg, and/or a mean arterial pressure of at least about 88 mmHg. In some embodiments, a carotid FCWI range (mmHg m/s³) of at least about 10,000, a systolic blood pressure (mmHg) of at least about 135 mmHg, a pulse pressure of at least about 55 mmHg, a diastolic blood pressure of at most about 75 mmHg, and/or a mean arterial pressure of at least about 95 mmHg. Without intending to be bound by any particular dose or administration regimen, a therapeutically effective dose is an amount of the biologic therapeutic agent that, when administered to the subject in need thereof, treats or at least partially treats, reduces the effects of, or at least partially reduces the effects of, the subject’s condition (e.g., neurodegenerative condition, cognitive decline). The therapeutically effective dose for each biologic therapeutic agent is selected based upon a variety of factors, including but not limited to, one or more characteristics of the biologic therapeutic agent (e.g., bioactivity, pharmacokinetics, pharmacodynamics, and bioavailability), the physiological condition of the subject (e.g., age, sex, disease type and stage, general physical condition, responsiveness to a given dosage, and type of medication), and the route of administration.

### A. Cell-Based Biologic Therapeutic Agents

Cell-based biologic therapeutic agents enhance neuroplasticity, promote neurogenesis, regenerate neurons, promote angiogenesis, reduce inflammation, reduce immune responses, regenerate immune cells, and/or regenerate supporting tissue. In some embodiments, the cell-based biologic therapeutic agent includes cells produced using a therapeutic agent that stimulates stem cell proliferation, neurogenesis, neurovascular remodeling, or cerebral blood flow (e.g., IRL-1620 and other endothelin B (ETB) receptor agonists).

In other embodiments, cell-based biologic therapies include, but are not limited to, stem cell therapies, progenitor and/or precursor cell therapies, extracellular vesicle therapies, and genetically modified immune cell therapies. In some embodiments, cells useful in cell-based biologic therapies may be autologous cells or may be allogenic cells. In certain embodiments, cells useful in cell-based biologic therapies can be modified prior to delivery to a subject. Non-limiting examples of modifications include ex vivo propagation with one or more factors, such as a chemokine, a cytokine, a growth factor, or other agent, and genetic engineering. Cells can be genetically engineered to express a non-native peptide, overexpress a native peptide, or a combination thereof. Peptides that cells can be engineered to express include but are not limited to glial cell-derived neurotrophic factor (GDNF), nerve growth factor (NGF), and brain derived neurotrophic factor (BDNF). Cells can also, or instead, be genetically engineered to alter expression of a peptide by modifying one or more nucleic acids in the cell, such as by correcting a point mutation. Genetic engineering techniques include gene editing, gene targeting, CRISPR, TALENs, zinc-finger nucleases, or the like. Genetically engineered extracellular vesicles (e.g., exosomes) are vesicles derived from genetically engineered cells and are also an example of cell-based biologic agents.

### 1. Stem Cell Therapies

In some embodiments, stem cells include, but are not limited to, cells having self-renewing, totipotent, pluripotent, and/or multipotent potential. Examples of stem cells include, but are not limited to, embryonic stem cells, naive stem cells, somatic stem cells, adult stem cells, mesenchymal stem cells, hematopoietic stem cells, neural stem cells, induced pluripotent stem cells, and genetically modified stem cells. Markers (e.g., peptides) expressed by stem cells of interest in the present disclosure include, but are not limited to, SSEA-4, TRA-1-60, TRA-1-81, OCT4, SOX2, CD90, CD34, CD133, CD73, and CD105. Examples of stem cell therapies include single-infusion allogeneic human mesenchymal stem cells (hMSCs; Stemedica Cell Technologies, Inc.), multiple-infusion allogeneic hMSCs (University of Miami), hMSCs (Longeveron), autologous adipose-derived HB-adMSCs (Hope Biosciences), AstroStem® (autologous ad-MSCs; Nature Cell Co. Ltd.), Neurostem® (human umbilical cord blood derived mesenchymal stem cells; Medipost Co. Ltd.), and CB-AC-02 (placenta derived MSCs; CHA Biotech).

Stem cell therapies are administered at a dose effective to treat the subject’s neurological condition and/or prevent onset of a neurological condition. In some embodiments, the stem cell therapies are administered to a subject who has been identified as likely to develop the neurological condition (e.g., Alzheimer’s disease). For example, the identified subject may be identified as likely to develop the condition in one year, two years, five years, ten years, fifteen years, or more. Dosages can be administered in one or more administrations or by continuous infusion. Doses range from about 1 million to about 250 million stem cells. In some embodiments, the dose is about 10 million to about 200 million stem cells, about 15 million to about 150 million stem cells, or about 20 million to about 100 million stem cells.

### 2. Progenitor And/Or Precursor Cell Therapies

Progenitor cells or precursor cells are cells which are not fully differentiated, but also not totipotent or pluripotent. Examples of progenitor or precursor cells include, but are not limited to, endothelial progenitor cells and neural progenitor cells. Markers expressed by progenitor or precursor cells of interest in the present disclosure include, but are not limited to, CD34, VEGFR2, and CD133.

Progenitor cell or precursor cell therapies are administered at a dose effective to treat the subject’s neurological condition and/or prevent onset of a neurological condition. Dosages can be administered in one or more administrations or by continuous infusion. Doses range from about 1 million to about 250 million progenitor or precursor cells. In some embodiments, the dose is about 10 million to about 200 million progenitor or precursor cells, about 15 million to about 150 million progenitor or precursor cells, or about 20 million to about 100 million progenitor or precursor cells.

### 3. Extracellular Vesicle Therapies

Extracellular vesicles are lipid-bilayer delimited and released from any type of cell, such as from stem cells. Extracellular vesicles (e.g., microvesicles) can deliver a molecule from stem cells with lower potential of immune reactions compared to synthetic vesicles and/or vesicles derived from other sources. Additionally, microvesicles may be able to cross the blood brain barrier. Examples of extracellular vesicles include, but are not limited to, stem cell derived microvesicles, exosomes, and ectosomes. Extracellular vesicles can contain one or more molecules that can be delivered to a target, such as a second cell different from a first cell that released the extracellular vesicle. Non-limiting examples of molecules contained by an extracellular vesicle include a nucleic acid (e.g., RNA), a cytokine, a growth factor, a signaling lipid, or other therapeutic agent such as a biologic drug.

Extracellular vesicle therapies are administered at a dose effective to treat the subject’s neurological condition and/or prevent onset of a neurological condition. Dosages can be administered in one or more administrations or by continuous infusion. Doses range from about 1 million to about 250 million extracellular vesicles. In some embodiments, the dose is about 10 million to about 200 million extracellular vesicles, about 15 million to about 150 million extracellular vesicles, or about 20 million to about 100 million extracellular vesicles.

### 4. Immune Cell Therapies

Immune cells are white blood cells and, in some embodiments, can be genetically engineered to express a non-native peptide, such as a peptide not normally expressed by a white blood cell, including but not limited to wild-type peptides and polypeptides and synthetic peptides and polypeptides. Examples of genetically modified immune cells include, but are not limited to, T cells and/or phagocytes which target peptides associated with Alzheimer’s disease, such as Alzheimer’s biomarkers including, but not limited to, β-amyloid. Genetically modified cells include CAR-modified immune cells, such as T cells and NK cells (CAR-T cells and NK-cells), and TCR-modified cells, and immune cells modified to express one or more factors, such as BDNF. For example, CD4⁺ T cells engineered to express BDNF can migrate to a location within the subject having a β-amyloid deposit. In some embodiments, CD4⁺T cells are isolated following β-amyloid exposure, engineered to express BDNF, and then delivered to the subject. In some embodiments, immune cells that migrate to a location within the subject having the β-amyloid deposit, a Tau deposit, a fibrin deposit, a fibrinogen deposit, a heme deposit, or a combination thereof, can secrete an antibody that binds to β-amyloid, tau, fibrin, fibrinogen, or heme. In the embodiments, immune cells secreting the antibody can be genetically modified to express a CAR, can be identified and/or developed by one or more immunization methods.

Immune cell therapies are administered at a dose effective to treat the subject’s neurological condition and/or prevent onset of a neurological condition. Dosages can be administered in one or more administrations or by continuous infusion. Doses range from about 1 million to about 250 million immune cells. In some embodiments, the dose is about 10 million to about 200 million immune cells, about 15 million to about 150 million immune cells, or about 20 million to about 100 million immune cells.

### B. Gene-Based Therapeutic Agents

Gene-based therapeutic agents include agents formed of genetic material (e.g., DNA or RNA) suitable for transfer into a host. Expression of the transferred genetic material is induced within the host, and/or expression of target genetic material within the host is reduced, prevented, or otherwise eliminated following transfer of the genetic material. Gene-based therapeutic agents thereby augment expression or regulation of a gene, suppress expression of a gene, and/or edit at least a portion of the host’s genome (e.g., using TALENs, zinc-finger nucleases, CRISPR, or the like). In some embodiments, gene-based therapeutic agents include transgenes carried by viral vectors and non-viral vectors. Transgenes are nucleic acids that encode a non-native gene or disrupt expression of a wild-type gene that include, but are not limited to, nerve growth factor (NGF), telomerase reverse transcriptase (hTERT), apolipoprotein E2 (APOE2), β-arrestin-2, genes encoding at least a portion of an antibody, a neurotrophic factor, a TALEN, a zinc-finger nuclease, a CRISPR-Cas9 cassette, a microRNA, and an siRNA.

### 1. Viral Vector-Based Therapeutic Agents

A viral vector refers to a virus particle engineered to genetically modify a host (e.g., cell, such as a mammalian cell) in vivo or ex vivo. Types of viruses that viral vectors can be isolated from, or derived from, and otherwise modified include, but are not limited to, adeno-associated virus (AAV), adenovirus, lentivirus, and retrovirus. A non-exhaustive list of viral vector-based therapeutic agents includes AAVrh.10hAPOE2, serotype rh.10 AAV gene transfer (Weill Medical College of Cornell University), CERE-110 (AAV delivery of NGF, Sangamo Therapeutics), and AAV-hTERT (Libella Gene Therapeutics).

While viral vector-based therapeutic agents can be administered at any therapeutically effective dose that is effective to treat the subject in need thereof, doses range from about 0.0001 to about 500 mg/kg of body weight. For example, dosages are between about 0.1 mg/kg and about 500 mg/kg, between about 0.1 mg/kg and about 250 mg/kg, between about 0.1 mg/kg and about 100 mg/kg, between about 0.1 mg/kg and about 50 mg/kg, or between about 0.1 mg/kg and about 25 mg/kg. For example, dosages also include one or more doses of about 0.1 mg/kg, about 0.2 mg/kg, about 0.3 mg/kg, about 0.4 mg/kg, about 0.5 mg/kg, about 1.0 mg/kg, about 1.5 mg/kg, about 2.0 mg/kg, about 3.0 mg/kg, about 4.0 mg/kg, about 5.0 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 /mg/kg, about 100 mg/kg, about 200 mg/kg, about 300 mg/kg, about 400 mg/kg, or about 500 mg/kg (or any combination thereof). In some embodiments, the viral vector-based therapeutic agents are administered at a flat dose of about 1 mg, about 5 mg, about 10 mg, about 25 mg, about 50 mg, about 100 mg, about 200 mg, about 300 mg, about 500 mg, about 1000 mg, or higher. For example, the viral vector-based therapy is administered in one to fifty doses (e.g., the therapy may be delivered in a single dose, in two doses, in three doses, in four doses, in five doses, etc.). In some embodiments, the total dose administered is in the range of about 25 mg to about 5000 mg or higher, of about 50 mg to about 2500 mg, of about 50 mg to about 2000 mg, about 50 mg to about 1500 mg, about 50 mg to about 1000 mg, about 50 mg to about 500 mg, about 50 mg to about 100 mg, or any other range having a therapeutic effect on the subject’s condition. For example, the total dose administered can be about 25 mg, about 50 mg, about 100 mg, about 200 mg, about 300 mg, about 400 mg, about 500 mg, about 600 mg, about 700 mg, about 1000 mg, about 1200 mg, about 1500 mg, about 1800 mg, about 2000 mg, about 2500 mg, about 3000 mg, about 4000 mg, about 5000 mg, or higher. In some embodiments, the viral vector-based therapy is administered chronically. In some embodiments, dosages of viral vector-based therapy are administered in one or more separate administrations or by continuous infusion.

### 2. Non-Viral Vector-Based Therapeutic Agents

A non-viral vector refers to a nucleic acid that is bare and packaged using a delivery system rather than carried by a viral vector. Bare nucleic acids include DNA and RNA. Suitable delivery systems for packaging include (1) vesicles and nanoparticles, such as but not limited to, exosomes, lipoplexes, polyplexes, and gold nanoparticles; (2) carrier compounds, including but not limited to, DNA-binding carrier proteins, and (3) bactofection. Suitable routes of administration include those described herein, and in particular but not limited to, infusion (e.g., intravenous infusion) and injection (e.g., site-specific injection), such as subcutaneous, intravenous, intramuscular, and intracerebral.

While non-viral vector-based therapeutic agents can be administered at any therapeutically effective dose that is effective to treat the subject in need thereof, doses range from about 0.0001 to about 500 mg/kg of body weight. For example, dosages are between about 0.1 mg/kg and about 500 mg/kg, between about 0.1 mg/kg and about 250 mg/kg, between about 0.1 mg/kg and about 100 mg/kg, between about 0.1 mg/kg and about 50 mg/kg, or between about 0.1 mg/kg and about 25 mg/kg. For example, dosages also include one or more doses of about 0.1 mg/kg, about 0.2 mg/kg, about 0.3 mg/kg, about 0.4 mg/kg, about 0.5 mg/kg, about 1.0 mg/kg, about 1.5 mg/kg, about 2.0 mg/kg, about 3.0 mg/kg, about 4.0 mg/kg, about 5.0 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 /mg/kg, about 100 mg/kg, about 200 mg/kg, about 300 mg/kg, about 400 mg/kg, or about 500 mg/kg (or any combination thereof). In some embodiments, the non-viral vector-based therapeutic agents are administered at a flat dose of about 1 mg, about 5 mg, about 10 mg, about 25 mg, about 50 mg, about 100 mg, about 200 mg, about 300 mg, about 500 mg, about 1000 mg, or higher. For example, the non-viral vector-based therapy is administered in one to fifty doses (e.g., the therapy may be delivered in a single dose, in two doses, in three doses, in four doses, in five doses, etc.). In some embodiments, the total dose administered is in the range of about 25 mg to about 5000 mg or higher, of about 50 mg to about 2500 mg, of about 50 mg to about 2000 mg, about 50 mg to about 1500 mg, about 50 mg to about 1000 mg, about 50 mg to about 500 mg, about 50 mg to about 100 mg, or any other range having a therapeutic effect on the subject’s condition. For example, the total dose administered can be about 25 mg, about 50 mg, about 100 mg, about 200 mg, about 300 mg, about 400 mg, about 500 mg, about 600 mg, about 700 mg, about 1000 mg, about 1200 mg, about 1500 mg, about 1800 mg, about 2000 mg, about 2500 mg, about 3000 mg, about 4000 mg, about 5000 mg, or higher. In some embodiments, the non-viral vector-based therapy is administered chronically. In some embodiments, dosages of non-viral vector-based therapy are administered in one or more separate administrations or by continuous infusion.

### C. Microbiome-Based Therapeutic Agents

Microbiome agents include, but are not limited to, microbiome transplants, probiotics, isolated bacterial strain(s), phages, and therapeutic agents (e.g., drugs). Examples of microbiome-based biologic therapeutic agents include fecal microbiota transplant (University of Wisconsin, Madison), Omni-Biotic Stress Repair (Bifidobacterium bifidum W23, B. lactis W51, B. lactis W52, Lactobacillus acidophilus W22, L. casei W56, L. paracasei W20, L. plantarum W62, L. salivarius W24, Lactococcus lactis W19, 7.5 x 10⁹ Colony Forming Units/g twice daily dissolved in water; Medical University of Graz), and GV-971 (sodium oligo-mannurarate remodeling of gut microbiome, Green Valley Pharmaceutical Co).

While microbiome biologic therapeutic agents can be administered at any therapeutically effective dose that is effective to treat the subject in need thereof, doses range from about 0.0001 to about 500 mg/kg of body weight. For example, dosages are between about 0.1 mg/kg and about 500 mg/kg, between about 0.1 mg/kg and about 250 mg/kg, between about 0.1 mg/kg and about 100 mg/kg, between about 0.1 mg/kg and about 50 mg/kg, or between about 0.1 mg/kg and about 25 mg/kg. For example, dosages also include one or more doses of about 0.1 mg/kg, about 0.2 mg/kg, about 0.3 mg/kg, about 0.4 mg/kg, about 0.5 mg/kg, about 1.0 mg/kg, about 1.5 mg/kg, about 2.0 mg/kg, about 3.0 mg/kg, about 4.0 mg/kg, about 5.0 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 /mg/kg, about 100 mg/kg, about 200 mg/kg, about 300 mg/kg, about 400 mg/kg, or about 500 mg/kg (or any combination thereof). In some embodiments, the microbiome biologic immunotherapy is administered at a flat dose of about 1 mg, about 5 mg, about 10 mg, about 25 mg, about 50 mg, about 100 mg, about 200 mg, about 300 mg, about 500 mg, about 1000 mg, or higher. For example, the microbiome biologic therapy is administered in one to fifty doses (e.g., the therapy may be delivered in a single dose, in two doses, in three doses, in four doses, in five doses, etc.). In some embodiments, the total dose administered is in the range of about 25 mg to about 5000 mg or higher, of about 50 mg to about 2500 mg, of about 50 mg to about 2000 mg, about 50 mg to about 1500 mg, about 50 mg to about 1000 mg, about 50 mg to about 500 mg, about 50 mg to about 100 mg, or any other range having a therapeutic effect on the subject’s condition. For example, the total dose administered can be about 25 mg, about 50 mg, about 100 mg, about 200 mg, about 300 mg, about 400 mg, about 500 mg, about 600 mg, about 700 mg, about 1000 mg, about 1200 mg, about 1500 mg, about 1800 mg, about 2000 mg, about 2500 mg, about 3000 mg, about 4000 mg, about 5000 mg, or higher. In some embodiments, the microbiome biologic immunotherapy is administered chronically. In some embodiments, dosages of microbiome biologic therapy are administered in one or more separate administrations or by continuous infusion.

### D. Peptide-Based Therapeutic Agents

Peptide-based therapeutic agents include, but are not limited to, peptides, proteins, derivatives, and/or variants thereof, provided to the subject or otherwise produced by the subject, such as following administration of a biologic therapeutic agent described herein. The peptide-based therapeutic agents used in accordance with the embodiments described herein may be isolated from a biologic sample, recombinantly produced, or synthetically produced. Examples of peptide-based biologic therapeutic agents that can be used in accordance with the combination therapies described herein include, but are not limited to, peptides that have a protective effect on the lining of blood vessels in the brain and/or that repair damage to blood vessels caused by a disease or condition.

In some embodiments, the peptide-based biologic therapeutic agent is a peptide that stimulates stem cell proliferation, neurogenesis, neurovascular remodelling, or increased cerebral blood flow. In certain such embodiments, the peptide-based biologic therapeutic agent may be an endothelin B (ETB) receptor agonist such as IRL-1620 (a peptide also known as sovateltide, SPI-1620, and PMZ-1620). Activation of ETB receptors with IRL-1620 produces neurovascular repair and remodeling or neuroregeneration. There are dormant or hidden stem cells in the brain, which become active following injury to the brain. Similarly, there are neuronal progenitor cells in the adult brain that continue to form new neurons throughout life, often helping to repair and restore function in the case of disease or trauma. As such, activation of ETB receptors can promote proliferation and migration of endothelial cells and progenitors, which may repair damage to the blood brain barrier (BBB). Further, intravenous administration of IRL-1620 (sovateltide) augments the activity of neuronal progenitor cells in the brain to repair damage by formation of new mature neurons and blood vessels. In addition, IRL-1620 has anti-apoptotic activity and also increases cerebral blood flow. As such, IRL-1620 and other such agents act to co-stimulate the production of cell-based biologic therapeutic agents by acting to stimulate production of the cells that ultimately treat damage caused by a condition.

In other embodiments, the peptide-based biologic therapeutic agent is activated protein kinase C (APC), a portion thereof, a derivative thereof, and/or a variant thereof. In some embodiments, the APC, portion thereof, derivative thereof, and/or variant thereof is 3K3A-APC (ZZ Biotech, LLC). Protein Kinase C (PKC) is an important mediator of vascular permeability. Low-levels of PKC activation can lead to a substantial change in endothelial permeability, which often leads to inflammation and disease. Conversely, APC has cell-protecting, anti-inflammatory, and anti-coagulant properties that have an overall protective effect on the lining of blood vessels in the brain.

While peptide-based biologic therapeutic agents can be administered at any therapeutically effective dose that is effective to treat the subject in need thereof, doses range from about 0.0001 to about 500 mg/kg of body weight. For example, dosages are between about 0.1 mg/kg and about 500 mg/kg, between about 0.1 mg/kg and about 250 mg/kg, between about 0.1 mg/kg and about 100 mg/kg, between about 0.1 mg/kg and about 50 mg/kg, or between about 0.1 mg/kg and about 25 mg/kg. For example, dosages also include one or more doses of about 0.1 mg/kg, about 0.2 mg/kg, about 0.3 mg/kg, about 0.4 mg/kg, about 0.5 mg/kg, about 1.0 mg/kg, about 1.5 mg/kg, about 2.0 mg/kg, about 3.0 mg/kg, about 4.0 mg/kg, about 5.0 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 /mg/kg, about 100 mg/kg, about 200 mg/kg, about 300 mg/kg, about 400 mg/kg, or about 500 mg/kg (or any combination thereof). In some embodiments, the peptide-based biologic immunotherapy is administered at a flat dose of about 1 mg, about 5 mg, about 10 mg, about 25 mg, about 50 mg, about 100 mg, about 200 mg, about 300 mg, about 500 mg, about 1000 mg, or higher. For example, the peptide-based biologic therapy is administered in one to fifty doses (e.g., the therapy may be delivered in a single dose, in two doses, in three doses, in four doses, in five doses, etc.). In some embodiments, the total dose administered is in the range of about 25 mg to about 5000 mg or higher, of about 50 mg to about 2500 mg, of about 50 mg to about 2000 mg, about 50 mg to about 1500 mg, about 50 mg to about 1000 mg, about 50 mg to about 500 mg, about 50 mg to about 100 mg, or any other range having a therapeutic effect on the subject’s condition. For example, the total dose administered can be about 25 mg, about 50 mg, about 100 mg, about 200 mg, about 300 mg, about 400 mg, about 500 mg, about 600 mg, about 700 mg, about 1000 mg, about 1200 mg, about 1500 mg, about 1800 mg, about 2000 mg, about 2500 mg, about 3000 mg, about 4000 mg, about 5000 mg, or higher. In some embodiments, the peptide-based biologic immunotherapy is administered chronically. In some embodiments, dosages of peptide-based biologic therapy are administered in one or more separate administrations or by continuous infusion.

One skilled in the art will understand that the foregoing therapies and accompanying description is for illustrative purposes and does not limit the therapies that may be provided in certain embodiments of the present technology. Accordingly, any therapy useful in or designed to treat a neurological condition, such as a neurodegenerative condition, may be present in certain embodiments of the present technology.

### V. Selected Methods of Treating Neurological Conditions With a Combination Of An Implantable Damping Device and a Biologic Therapeutic Agent

Reducing a subject’s pulse pressure with the implantable damping devices has subsequent downstream impacts on other factors that contribute to onset, duration, and/or progression of the subject’s condition (e.g., neurological condition). Combining use of devices of the present disclosure to reduce the subject’s pulse pressure with biologic therapeutic agents that, when administered to the subject, enhance an effect of reduced pulse pressure on certain outcomes results in a greater reduction and prevention of onset, duration, and/or progression of the subject’s neurological condition. These outcomes are, for example but are not but not limited to, increased expression of sRAGE, decreased expression and/or accumulation of Tau protein, decreased levels of plasma Aβ, decreased presence of Aβ in vessel walls and in the subject’s brain, increased numbers and persistence of stem cells, progenitor cells, and precursor cells, increased expression and transmission of neurotrophic factors (e.g., neurotrophic cell signalling pathways), reduced or prevented expression of APOE4, and increased a normal biome and/or reduced or prevented an abnormal biome. These factors, in addition to others, contribute to inflammation (systemic inflammation, blood brain barrier inflammation, neuroinflammation), oxidative stress, ischemia, blood brain barrier dysfunction or permeability, detrimental neural exosomes, and aging which subsequently cause synaptic and/or neuronal dysfunction and impaired neurotransmission. Accordingly, reducing these outcomes results in reduced inflammation (e.g., neuroinflammation, systemic inflammation, blood brain barrier inflammation), decreasing reactive oxygen species (e.g., oxidative stress), reduced ischemia, restoring blood brain barrier integrity, reduced detrimental neural exosomes, and improving the impacts of aging which subsequently cause synaptic and/or neuronal dysfunction and impaired neurotransmission. This occurs in subjects suffering from conditions such as progressive cognitive dysfunction and dementia. Without intending to be limiting, subjects having a FCWI value in the top quartile are thought to benefit from a reduction in the FCWI, systolic acceleration time, systolic pressure, and/or pulse pressure value achieved with the implantable damping devices of the present disclosure.

Several biological pathways, for example such as those described herein, may contribute to a neurological condition (e.g., dementia). Without intending to be bound by any particular theory, it is thought that interfering (e.g., altering, effecting, impairing, inhibiting, reducing, or otherwise changing the function of) two or more biological pathways is more effective for treating, preventing, or otherwise reducing the subject’s neurological condition, and/or symptoms thereof, rather than interfering with a single biological pathway. In this way, the effects of combining the implantable damping device and at least one biologic therapeutic agent of the present technology may be complementary, additive or even synergistic when compared to an effect of the implantable damping device and the biologic therapeutic agent alone. Accordingly, combining the implantable damping devices with one or more biologic therapeutic agents that affect these other factors further treats and/or slows one or more effects of the condition.

As described above, combinatorial therapies of the present technology include an implantable damping device and a biologic therapeutic agent for treating and/or preventing the progression of the condition. Some embodiments of the present technology, for example, are directed to combinatorial therapies including the implantable damping devices described above under Headings I-III and one or more biologic therapeutic agents that target these factors. Some of these therapeutic agents are described above under Heading IV and include, but are not limited to, stem cell therapies, gene-based therapies, and microbiome-based therapies. When combined, the implantable damping devices and biologic therapeutic agents of the present technology have a greater effect on treating and/or preventing one or more aspects of the condition upon a subject when compared either to the effects of the implantable damping device or biologic therapeutic agent alone. For example, providing an implantable damping device that reduces the subject’s pulse pressure and a stem cell-based therapy that reduces systemic inflammation and promotes neurogenesis in the subject’s brain and blood vessel walls improves synaptic and/or neuronal function and neurotransmission, thereby treating and/or preventing progressive cognitive dysfunction and dementia. Subjects who have elevated pulse pressure or subjects that would otherwise benefit from having reduced pulse pressure may also have a blood brain barrier with increased permeability, one or more microbleeds, increased inflammation, increased oxidative stress, increased levels of circulating or CSF inflammatory cytokines and/or immune cells, increased levels of reactive oxygen species, decreased levels of circulating neurotrophic factors, expression of the APOE4 allele, or reduced levels of APOE2 or APOE3 protein, previously been provided one or more chemotherapeutic agents, or a combination thereof. The implantable damping device can be provided to these subjects having elevated pulse pressure or subjects that would otherwise benefit from having reduced pulse pressure before, after, or concurrently with a biologic therapeutic agent, such as stem cell therapies, gene-based therapies, microbiome-based therapies, and peptide-based therapies.

Methods of the present disclosure include methods for treating a patient having a condition including the steps of, (a) determining or having determined whether the patient has an elevated pulse pressure or elevated pulse wave intensity/FCWI and a history of blood brain barrier dysregulation or permeability, decreasing reactive oxygen species (e.g., oxidative stress), decreasing microbleeds, decreasing inflammation (e.g., systemic inflammation, blood brain barrier inflammation, neuroinflammation), decreasing the level of at least one circulating or cerebral spinal fluid (CSF) cytokine, or a combination thereof by, (i) obtaining or having obtained information which indicates that the patient has the elevated pulse pressure or elevated pulse wave intensity/FCWI and has previously had symptoms of blood brain barrier dysregulation or permeability, oxidative stress, microbleeds, systemic inflammation, blood brain barrier inflammation, neuroinflammation, increased level of at least one circulating or CSF cytokine, or a combination thereof and/or was previously diagnosed with a microbleed, systemic inflammation, blood brain barrier inflammation, neuroinflammation, increased level of at least one circulating or CSF cytokine, or a combination thereof; and/or (ii) monitoring or having monitored the subject for the elevated pulse pressure or elevated pulse wave intensity/FCWI and symptoms of blood brain barrier dysregulation or permeability, oxidative stress, a microbleed, systemic inflammation, blood brain barrier inflammation, neuroinflammation, increased level of at least one circulating or CSF cytokine, or a combination thereof. In some embodiments, the methods also include, (b) if the patient has previously had symptoms of blood brain barrier dysregulation or permeability, oxidative stress, a microbleed, systemic inflammation, blood brain barrier inflammation, neuroinflammation, increased level of at least one circulating or CSF cytokine, or a combination thereof, was previously diagnosed with a microbleed, systemic inflammation, blood brain barrier inflammation, neuroinflammation, increased level of at least one circulating or CSF cytokine, or a combination thereof, and/or symptoms of blood brain barrier dysregulation or permeability, oxidative stress, a microbleed, systemic inflammation, blood brain barrier inflammation, neuroinflammation, increased level of at least one circulating or CSF cytokine, or a combination thereof were monitored, then (i) providing a cell-based therapy, a gene-based therapy, a microbiome-based therapy, or a peptide-based therapy to the patient, and (ii) providing a device for treating and/or preventing one or more effects of the condition. In some embodiments, the methods further include, (c) if the patient has not had symptoms of blood brain barrier dysregulation or permeability, oxidative stress, a microbleed, systemic inflammation, blood brain barrier inflammation, neuroinflammation, increased level of at least one circulating or CSF cytokine, or a combination thereof, was not previously diagnosed with blood brain barrier dysregulation or permeability, oxidative stress, a microbleed, systemic inflammation, blood brain barrier inflammation, neuroinflammation, increased level of at least one circulating or CSF cytokine, or a combination thereof, then providing the device for treating and/or preventing one or more effects of the condition.

Additional methods of the present disclosure include methods for treating a patient having a condition including the steps of, (a) determining or having determined whether the patient has an elevated pulse pressure or pulse wave intensity/FCWI and a history of a reduced level of one or more circulating neurotrophic factors by (i) obtaining or having obtained information which indicates that the patient has the elevated pulse pressure or pulse wave intensity/FCWI and the reduced level of one or more circulating neurotrophic factors and/or was previously diagnosed with the reduced level of one or more circulating neurotrophic factors; and/or (ii) monitoring or having monitored the subject for arterial stiffening, the elevated pulse pressure, elevated pulse wave intensity/FCWI, and the reduced level of one or more circulating neurotrophic factors. In some embodiments, the methods also include, (b) if the patient has previously had a reduced level of one or more circulating neurotrophic factors, was previously diagnosed with a reduced level of one or more circulating neurotrophic factors, and/or a reduced level of one or more circulating neurotrophic factors was monitored, then (i) providing a cell-based therapy to the patient, and (ii) providing a device for treating and/or preventing one or more effects of the condition. In some embodiments, the methods further include, (c) if the patient has not had a reduced level of one or more circulating neurotrophic factors, was not previously diagnosed with a reduced level of one or more circulating neurotrophic factors, then providing the device for treating and/or preventing one or more effects of the condition.

Further methods of the present disclosure include methods for treating a patient having a condition including the steps of, (a) determining or having determined whether the patient has an elevated pulse pressure, elevated pulse wave intensity/FCWI, and/or the APOE4 allele by (i) obtaining or having obtained information which indicates that the patient has the elevated pulse pressure, elevated pulse wave intensity/FCWI, and has previously been positive for the APOE4 allele and/or was previously diagnosed with the expression of APOE4; and/or (ii) monitoring or having monitored the subj ect for the elevated pulse pressure and the expression of APOE4. In some embodiments, the methods also include, (b) if the patient has previously had the expression of APOE4, was previously diagnosed with the\expression of APOE4, and/or the increased level of APOE4 was monitored, then (i) providing a gene-based therapy to the patient, and (ii) providing a device for treating and/or preventing one or more effects of the condition. In some embodiments, the methods further include, (c) if the patient has not had symptoms of the APOE4 allele/variant, was not previously diagnosed with the APOE4 allele, then providing the device for treating and/or preventing one or more effects of the condition.

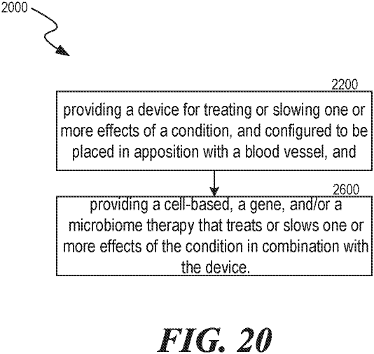

FIG. 20 is a flow chart illustrating method 2000 for treating and/or preventing one or more effects of a subject’s condition. At block 2200, the method 2000 provides a device for treating and/or preventing one or more effects of the condition. The device is the implantable damping devices of the present technology and is configured to be placed in apposition with the subject’s blood vessel. Similar to other devices of the present technology, the device provided in method 2000 includes the flexible damping member having both the inner surface formed of the sidewall having one or more at least partially deformable portions and the outer surface. In addition, the abating substance is disposed within the partially deformable portions and is configured to move longitudinally and/or radially therein in response to pulsatile blood flow within the blood vessel. At block 2600, the method 2000 provides a cell-based, a gene-based, a microbiome therapy, and/or a peptide-based therapy that treats or slows one or more effects of the condition in combination with the implantable damping device. In some embodiments, the cell-based, a gene-based, a microbiome therapy, and/or a peptide-based therapy is provided to the subject before the implantable damping device, up to about 24 hours, up to about 7 days, up to about 4 weeks, up to about 12 months, or up to about 5 years before the implantable damping device. In other embodiments, the implantable damping device is provided to the subject before the cell-based, a gene, and/or a microbiome therapy, up to about 24 hours, up to about 7 days, up to about 4 weeks, up to about 12 months, or up to about 5 years before the cell-based, a gene, and/or a microbiome therapy. For example, the cell-based, the gene-based, the microbiome therapy, the peptide-based therapy, and/or the implantable damping device is provided to the subject about 0 to about 24 hours, about 1 to about 20 hours, about 3 to about 12 hours, about 5 to about 10 hours, about 1 day to about 7 days, about 2 days to about 6 days, about 3 days to about 5 days, about 1 week to about 4 weeks, about 2 weeks to about 4 weeks, about 1 week to about 3 weeks, about 2 weeks to about 3 weeks, about 1 year to about 5 years, about 1 year to about 4 years, about 2 years to about 5 years, about 2 years to about 4 years, about 3 years to about 4 years, or about 4 years to about 5 years before the implantable damping device, the cell-based, the gene-based, the microbiome therapy, and/or the peptide-based therapy, respectively.

Steps of the foregoing method and additional methods disclosed herein can be performed in any order. For example, step (b) is performed after step (a) and before step (c). As another example, step (c) is performed after step (a) and before step (b).

As described above under Heading IV, the cell-based, a gene, and/or a microbiome therapy of the methods of the present technology is provided to the subject by administration.

Devices useful with the methods of the present technology include implantable damping devices of the present disclosure, such as devices comprising a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and an abating substance disposed within the one or more at least partially deformable portions of the sidewall configured to move longitudinally and/or radially within one partially deformable portion in response to pulsatile blood flow within the blood vessel.

When combined with the implantable damping devices of the present technology, the biologic therapeutic agents described herein are provided at a first dosage that is lower than a second dosage of the same biologic therapeutic agents provided in the absence of the implantable damping devices (e.g., subjects receiving only the biologic therapeutic agents rather than in combination with the implantable damping devices). For example, a subject having a neurodegenerative condition, such as dementia, is provided with a lower dose of Omni-Biotic before, during, or after being provided with the implantable damping device compared to a subject provided with a dose of Omni-Biotic without also being provided with the implantable damping device.

In some embodiments, when combined with the implantable damping devices of the present technology, the biologic therapeutic agents described herein are provided with a first dosing regimen which is less than a second dosing regimen of the same biologic therapeutic agents that is provided in the absence of the implantable damping devices. For example, a subject having a neurodegenerative condition, such as dementia, is provided with a first dosing regimen of Omni-Biotic before, during, or after being provided with the implantable damping device compared to a subject provided with a second dosing regimen of Omni-Biotic without also being provided with the implantable damping device.

In some embodiments, when combined with the implantable damping devices of the present technology, the biologic therapeutic agents described herein are provided with the biologic therapeutic agent by a first route which differs from a second route provided in the absence of the implantable damping devices. For example, a subject having a neurodegenerative condition, such as dementia, is provided with Omni-Biotic by the first route before, during, or after being provided with the implantable damping device compared to a subject provided with Omni-Biotic by the second route without also being provided with the implantable damping device. In some embodiments, the route of administration includes delivering the biologic therapeutic agent to the subject from the device, for example, by eluting the biologic therapeutic agent previously stored in at least a portion of the device.

### VI. Selected Systems for Treating Neurological Conditions With a Combination of An Implantable Damping Device and a Biologic Therapeutic Agent

In addition to the methods, damping devices, and biologic therapeutic agents described herein, the present technology also includes associated systems for treating and/or preventing one or more effects of the subject’s condition. Systems of the present technology include an effective amount of at least one therapy for treating and/or preventing one or more effects of the condition and a device for treating and/or preventing one or more effects of the condition. As explained above, devices of the present technology include at least a flexible damping member forming a generally tubular structure having an inner surface formed of a sidewall having one or more at least partially deformable portions, and an abating substance disposed within and configured to move longitudinally and/or radially within one partially deformable portion in response to pulsatile blood flow within the blood vessel. In some embodiments, the biologic therapy includes at least one or more biologic therapeutic agents that may be carried by the damping device. In these embodiments, the biologic therapeutic agent is disposed within and/or carried by at least one or more of the at least partially deformable portions of the damping device. When one or more of the at least partially deformable portions of the damping device are at least partially deformed, the effective amount of the therapeutic agent may be released from the device.

### VII. EXAMPLES

The following examples are illustrative of several embodiments of the present technology. The embodiments described herein are directed to treatment regimens that include a damping device (e.g., any of the devices described above in paragraphs [93] to [136]) and a biologic therapy (e.g., any biologic therapeutic agents discussed above in any of paragraphs [137]-[143]; cell-based therapy to include those discussed above in any of paragraphs [144] to [153]; any gene-based therapy to include those discussed above in any of paragraphs [154] to [158]; any microbiome therapy to include those discussed above in any of paragraphs [159] to [160]; and/or any peptide-based therapy to include those discussed above in any of paragraphs [161] to [165]). The treatment regimen is not limited to one biologic therapy and may include one or more additional biologic therapies. In accordance with some embodiments, the treatment regimen may be used in methods to treat or prevent a neurological condition using a combination therapy that includes steps of implanting or otherwise providing a damping device (e.g., any of the devices described above in paragraphs [93] to [136]) and administering or otherwise providing a biologic therapy (e.g., any biologic therapeutic agents discussed above in any of paragraphs [137]-[143]; cell-based therapy to include those discussed above in any of paragraphs [144] to [153]; any gene-based therapy to include those discussed above in any of paragraphs [154] to [158]; any microbiome therapy to include those discussed above in any of paragraphs [159] to [160]; and/or any peptide-based therapy to include those discussed above in any of paragraphs [161] to [165]). In such embodiments, the damping device may be implanted or provided before the biologic therapy, concurrently with the biologic therapy, or after the biologic therapy. One or more additional biologic therapies may also be administered at any time during the combination therapy. Non-limiting examples of the damping device, treatments using the damping device, combination treatments and treatment regimens—as well as studies and models that demonstrate the efficacy, benefits, and advantages of the treatments—are included in the examples below.

### A. Examples 1-16: Devices for Treating And/or Preventing the Progression Of Dementia

Example 1. A device for treating and/or preventing the progression of dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the arterial wall at the treatment site, thereby securing the damping member at the treatment site, and wherein the first and second anchoring members extend along only a portion of the length of the damping member such that at least a portion of the damping region is exposed between the first and second anchoring members and allowed to expand to a diameter greater than the deployed diameter.

Example 2. The device of example 1 wherein the damping member is configured to deform in response to a change in blood/pulse pressure or elevated pulse wave intensity/FCWI.

Example 3. The device of example 1 or example 2 wherein, at a location along the damping member coincident with a leading end of a pulse pressure wave, the distance between the inner surface and the outer surface of the damping member decreases in response to the pressure.

Example 4. The device of any one of examples 1-3 wherein the lumen of the damping member has an hourglass shape.

Example 5. The device of any one of examples 1-4 wherein the outer surface is generally cylindrical and the inner surface is undulating.

Example 6. The device of any one of examples 1-5 wherein each of the first and second anchoring members is an expandable stent.

Example 7. The device of any one of examples 1-5 wherein the each of the first and second anchoring members is an expandable mesh.

Example 8. The device of any one of examples 1-5 wherein each of the first and second anchoring members is at least one of an expandable stent and an expandable mesh.

Example 9. The device of any one of examples 1-8 wherein each of the first and second anchoring members is positioned around a circumference of the damping member.

Example 10. The device of any one of examples 1-8 wherein at least a portion of each of the first and second anchoring members is positioned within the damping member and extends through at least a portion of the thickness of the sidewall.

Example 11. The device of any one of examples 1-10 wherein the damping region is a first damping region, and wherein the damping member includes a plurality of damping regions between the first and second end portions.

Example 12. The device of any one of examples 1-11 wherein at least one of the first and second anchoring members comprise a plurality of fixation devices extending radially outwardly from the outer surface of the damping device.

Example 13. The device of any one of examples 1-12 wherein the device is configured to be positioned at a treatment site within the left common carotid artery.

Example 14. The device of any one of examples 1-13 wherein the device is configured to be positioned at a treatment site within the right common carotid artery.

Example 15. The device of any one of examples 1-14 wherein the device is configured to treat Alzheimer’s disease.

Example 16. The device of any one of examples 1-15 wherein the device is configured to reduce the occurrence of microbleeds in one or more branches of the artery downstream from the treatment site.

### B. Examples 17-31: Devices for Treating Dementia

Example 17. A device for treating dementia, comprising:
a damping member configured to be intravascularly positioned within an artery at a treatment site and having a lumen configured to direct blood flow to distal vasculature, the damping member being transformable between a low-profile state for delivery to the treatment site and an expanded state, wherein the damping member includes a damping region having a pressure limiter projecting laterally inwardly into the lumen to distribute pressure downstream from the damping member when a pulse pressure wave propagates along the damping member during systole; and
an anchoring member coupled to the damping member, wherein the anchoring member, in a deployed state, is configured to extend outwardly to a deployed diameter and contact a portion of the blood vessel wall at the treatment site, thereby securing the damping member at the treatment site, wherein the anchoring member extends along only a portion of the length of the damping member such that the damping region of the damping member is allowed to extend radially outward beyond the deployed diameter of the anchoring member.

Example 18. The device of example 17 wherein the damping member is configured to deform in response to a change in blood/pulse pressure or elevated pulse wave intensity/FCWI.

Example 19. The device of example 17 or example 18 wherein, at a location along the damping member coincident with a leading end of a pulse pressure wave, the distance between the inner surface and the outer surface of the damping member decreases in response to the pressure.

Example 20. The device of any one of examples 17-19 wherein the lumen of the damping member has an hourglass shape.

Example 21. The device of any one of examples 17-20 wherein the anchoring member is an expandable stent.

Example 22. The device of any one of examples 17-20 wherein the anchoring member is an expandable mesh.

Example 23. The device of any one of examples 17-20 wherein the anchoring member is at least one of an expandable stent and an expandable mesh.

Example 24. The device of any one of examples 17-23 wherein the anchoring member is positioned around a circumference of the damping member.

Example 25. The device of any one of examples 17-23 wherein at least a portion of the anchoring member is positioned within the damping member and extends through at least a portion of the thickness of the sidewall.

Example 26. The device of any one of examples 17-25 wherein the damping region is a first damping region, and wherein the damping member includes a plurality of damping regions between the first and second end portions.

Example 27. The device of any one of examples 17-26 wherein the anchoring member includes a plurality of fixation devices extending radially outwardly from the outer surface of the damping device.

Example 28. The device of any one of examples 17-27 wherein the device is configured to be positioned at a treatment site within the left common carotid artery.

Example 29. The device of any one of examples 17-28 wherein the device is configured to be positioned at a treatment site within the right common carotid artery.

Example 30. The device of any one of examples 17-29 wherein the device is configured to treat Alzheimer’s disease.

Example 31. The device of any one of examples 17-29 wherein the device is configured to reduce the occurrence of microbleeds in portions of the blood vessel downstream from the treatment site.

### C. Example 32: Devices for Treating Dementia

Example 32. A device for treating dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the blood vessel wall at the treatment site, thereby securing the damping member at the treatment site, and
wherein, when blood flows through the damping member during systole, the damping member absorbs a portion of the pulsatile energy of the blood, thereby reducing a magnitude of a pulse pressure transmitted to a portion of the blood vessel distal to the damping device.

### D. Examples 33-39: Devices for Treating a Blood Vessel

Example 33. A device for treating a blood vessel, comprising:
an anchoring system having a first portion and a second portion; and
a cushioning member located between the first and second portions of the anchoring system such that a portion of the cushioning member is not constrained by the anchoring system, and wherein the cushioning member is configured to absorb pulsatile energy transmitted by blood flowing with the vessel.

Example 34. The device of example 33 wherein the cushioning member is configured to expand in response to an increase of blood/pulse pressure or elevated pulse wave intensity/FCWI within the vessel, and relax as the blood/pulse pressure or elevated pulse wave intensity/FCWI within the vessel subsequently decreases.

Example 35. A device for treating a blood vessel, comprising:
an endovascular cushioning device having a proximal anchor and a distal anchor, each of the proximal and distal anchors being configured to abut against an inner wall of a major artery; and
an elastically deformable member extending between the proximal and distal anchors,
wherein the elastically deformable member is configured to expand in response to an increase of blood/pulse pressure or elevated pulse wave intensity/FCWI within the vessel, and relax as the blood/pulse pressure or elevated pulse wave intensity/FCWI within the vessel subsequently decreases.

Example 36. The device of example 35 wherein a portion of the elastically deformable membrane located longitudinally between the proximal and distal anchors defines a region of reduced internal cross-sectional area relative to the proximal and distal anchors when the elastically deformable membrane is radially relaxed.

Example 37. The device of example 35 or example 36 wherein the proximal and distal anchors are each radially expandable between a first diameter before deployment and a second diameter after deployment.

Example 38. The device of any one of examples 35-37, further comprising one or more threads secured to the proximal anchor.

Example 39. The device of example 38 wherein each thread is secured to an eyelet.

### E. Examples 40-43: Devices for Treating a Left Common Carotid Artery, a Right Common Carotid Artery, a Brachiocephalic Artery, the Ascending Aorta, An Internal Carotid Artery, Or An Abdominal Aorta

Example 40. A device for treating an artery selected from a left common carotid artery, a right common carotid artery, a brachiocephalic artery, the ascending aorta, an internal carotid artery, or an abdominal aorta, the device comprising:
a wrap fabricated from an elastically deformable material, and
an engagement formation adapted to secure two opposing edges of the wrap around the artery,
wherein the elastically deformable material is configured to radially expand during a systole stage and radially contract during a diastole stage.

Example 41. The device of example 40 wherein the engagement formation includes sutures and/or staples.

Example 42. The device of example 41 wherein the engagement formation includes a zip lock.

Example 43. A device for treating a left common carotid artery, a right common carotid artery, a brachiocephalic artery, or an ascending aorta, the device comprising:
a proximal anchor configured to be wrapped around the artery;
a distal anchor configured to be wrapped around the artery and longitudinally spaced relative to the proximal anchor; and
a helical band adapted to be wound around the artery, the helical band having a first end securable to the proximal anchor and an opposing second end securable to the distal anchor, wherein the helical band is adapted to radially expand during a systole stage and radially contract during a diastole stage.

### F. Examples 44-85: Devices for Treating And/or Preventing The Effects Of Dementia

Example 44. A device for treating and/or preventing the effects of dementia, comprising:

a damping member having a low-profile state and a deployed state, wherein, in the deployed state, the damping member comprises a deformable, generally tubular sidewall having an outer surface and an inner surface that is undulating in a longitudinal direction, and wherein the sidewall is configured to be positioned in apposition with a blood vessel wall to absorb pulsatile energy transmitted by blood flowing through the blood vessel.

Example 45. The device of example 44 wherein the damping member is configured to be positioned in apposition with at least one of a left common carotid artery, a right common carotid artery, and a brachiocephalic artery.

Example 46. The device of example 44 or example 45 wherein the damping member is configured to be positioned in apposition with an ascending aorta.

Example 47. The device of any one of examples 44-46 wherein the damping member is configured to be positioned in apposition with an inner surface of the blood vessel wall.

Example 48. The device of any one of examples 44-46 wherein the damping member is configured to be positioned in apposition with an outer surface of the blood vessel wall.

Example 49. The device of any one of examples 44-48 wherein the sidewall has an inner diameter, and, when the damping member is in a deployed state, the inner diameter increases then decreases in an axial direction.

Example 50. The device of any one of examples 44-49 wherein the cross-sectional area decreases then increases in longitudinal direction.

Example 51 The device of any one of examples 44-50 wherein the outer surface has a generally cylindrical shape.

Example 52. The device of any one of examples 44-50 wherein the outer surface has an undulating shape.

Example 53. The device of any one of examples 44-52, further comprising an anchoring member coupled to the damping member and axially aligned with only a portion of the damping member, wherein the anchoring member is configured to engage the blood vessel wall and secure the damping member to the blood vessel wall.

Example 54. The device of any one of examples 44-53 wherein the anchoring member is a first anchoring member and the device further comprises a second anchoring member coupled to the damping member, and wherein the second anchoring member:
is axially aligned with only a portion of the damping member, and
is spaced apart from the first anchoring member along the longitudinal axis of the damping member.

Example 55. The device of any one of examples 44-54 wherein, when the damping member is positioned adjacent the blood vessel wall, the damping member does not constrain the diameter of the blood vessel wall.

Example 56. A device for treating and/or preventing the effects of dementia, comprising:

an elastic member having a low-profile state for delivery to a treatment site at a blood vessel wall and a deployed state, wherein, in the deployed state, the elastic member is configured to abut an arterial wall and form a generally tubular structure having an inner diameter, an outer diameter, an outer surface, and an undulating inner surface, and wherein at least one of the outer diameter and the inner diameter increases and decreases in response to an increase and a decrease in pulse pressure within the blood vessel, respectively.

Example 57. The device of example 56 wherein the elastic member is configured to be positioned in apposition with at least one of a left common carotid artery, a right common carotid artery, and a brachiocephalic artery.

Example 58. The device of example 56 or example 57 wherein the elastic member is configured to be positioned in apposition with an ascending aorta.

Example 59. The device of any one of examples 56-58 wherein the elastic member is configured to be positioned in apposition with an inner surface of the blood vessel wall.

Example 60. The device of any one of examples 56-58 wherein the elastic member is configured to be positioned in apposition with an outer surface of the blood vessel wall.

Example 61. The device of any one of examples 56-60 wherein the sidewall has an inner diameter, and, when the elastic member is in a deployed state, the inner diameter increases then decreases in an axial direction.

Example 62. The device of any one of examples 56-61 wherein the cross-sectional area decreases then increases in longitudinal direction.

Example 63. The device of any one of examples 56-62 wherein the outer surface has a generally cylindrical shape.

Example 64. The device of any one of examples 56-62 wherein the outer surface has an undulating shape.

Example 65. The device of any one of examples 56-64, further comprising an anchoring member coupled to the elastic member and axially aligned with only a portion of the elastic member, wherein the anchoring member is configured to engage the blood vessel wall and secure the elastic member to the blood vessel wall.

Example 66. The device of example 65 wherein the anchoring member is a first anchoring member and the device further comprises a second anchoring member coupled to the elastic member, and wherein the second anchoring member:
is axially aligned with only a portion of the elastic member, and
is spaced apart from the first anchoring member along the longitudinal axis of the elastic member.

Example 67. The device of any one of examples 56-66 wherein, when the elastic member is positioned adjacent the blood vessel wall, the elastic member does not constrain the diameter of the blood vessel wall.

Example 68. A device for treating and/or preventing the effects of dementia, comprising:

a damping member including an abating substance, the damping member having a low-profile configuration and a deployed configuration, wherein, when the damping member is in the deployed configuration, the damping member forms a generally tubular structure configured to be positioned along the circumference of an artery such that, when a pulse wave traveling through the artery applies a stress at a first axial location along the length of the tubular structure, at least a portion of the abating substance moves away from the first location to a second axial location along the length of the tubular structure.

Example 69. The device of example 68, further comprising a structural element coupled to the damping member.

Example 70. The device of example 68 or example 69 wherein, in the deployed state, the damping member is configured to wrap around at least a portion of the circumference of the artery.

Example 71. The device of any one of examples 68-70 wherein, in the deployed state, the device has a pre-set helical configuration.

Example 72. The device of any one of examples 68-71 wherein the damping member includes a liquid.

Example 73. The device of any one of examples 68-72 wherein the damping member includes a gas.

Example 74. The device of any one of examples 68-73 wherein the damping member includes a gel.

Example 75. The device of any one of examples 68-74 wherein the damping member, in the deployed configuration, is configured to be positioned in apposition with an outer surface of the arterial wall.

Example 76. The device of any one of examples 68-74 wherein the damping member, in the deployed configuration, is configured to be positioned around the arterial wall such that an inner surface of the damping member is in contact with blood flowing through the artery.

Example 77. A device for treating and/or preventing the effects of dementia, comprising:
a damping member including a plurality of fluid particles, the damping member having a low-profile configuration and a deployed configuration, wherein, when the damping member is in the deployed configuration, the damping member is configured to be positioned along the circumference of an artery at a treatment site along a length of the artery,
wherein, when the damping member is in a deployed configuration and positioned at the treatment site, a wavefront traveling through the length of the artery redistributes at least a portion of the fluid particles along the length of the damping member such that the inner diameter of the damping member increases at the axial location along the damping member aligned with the wavefront while the inner diameter of the damping member at another axial location along the damping member decreases.

Example 78. The device of example 77, further comprising a structural element coupled to the damping member.

Example 79. The device of example 77 or example 78 wherein, in the deployed state, the damping member is configured to wrap around at least a portion of the circumference of the artery.

Example 80. The device of any one of examples 77-79 wherein, in the deployed state, the device has a pre-set helical configuration.

Example 81. The device of any one of examples 77-80 wherein the damping member includes a liquid.

Example 82. The device of any one of examples 77-81 wherein the damping member includes a gas.

Example 83. The device of any one of examples 77-82 wherein the damping member includes a gel.

Example 84. The device of any one of examples 77-83 wherein the damping member, in the deployed configuration, is configured to be positioned in apposition with an outer surface of the arterial wall.

Example 85. The device of any one of examples 77-84 wherein the damping member, in the deployed configuration, is configured to be positioned around the arterial wall such that an inner surface of the damping member is in contact with blood flowing through the artery.

### G. Examples 86-87: Methods for Treating And/Or Preventing the Effects of Dementia

Example 86. A method for treating and/or preventing the effects of dementia, comprising:

positioning a damping device in apposition with at least one of the brachiocephalic artery, the right common carotid artery, the left common carotid artery, the ascending aorta, and the aortic arch, the damping device comprising an elastic, generally tubular sidewall whereby the damping device absorbs pulsatile energy transmitted by blood flowing through the at least one of the brachiocephalic artery, the right common carotid artery, the left common carotid artery, the ascending aorta, and the aortic arch.

Example 87. A method for treating and/or preventing the effects of dementia, comprising:
positioning a damping device in apposition with the wall of an artery that delivers blood to the brain, the damping device comprising an elastic, generally tubular sidewall having an outer surface and an undulating inner surface; and
in response to a pulse pressure wave in blood flowing through the blood vessel, a contour of at least one of the inner surface and the outer surface changes.

### H. Example 88: Methods for Treating at Least One of the Brachiocephalic Artery, The Right Common Carotid Artery, The Left Common Carotid Artery, The Ascending Aorta, And The Aortic Arch

Example 88. A method for treating at least one of the brachiocephalic artery, the right common carotid artery, the left common carotid artery, the ascending aorta, and the aortic arch, the method comprising:
positioning a damping device in apposition with a blood vessel wall, the damping device comprising an elastic, generally tubular sidewall;
expanding at least one of the inner diameter and the outer diameter of the damping device in response to an increase in pulse pressure; and
contracting at least one of the inner diameter and the outer diameter of the damping device in response to a decrease in pulse pressure.

### I. Examples 89-96: Methods of Treating a Blood Vessel

Example 89. A method of treating a blood vessel, comprising:
inserting a catheter into a vessel and directing a tip of the catheter to a desired vascular location;
transferring a distal anchor from within the catheter tip into the vessel;
expanding the distal anchor such that a radially outer portion of the distal anchor engages with an inner wall of the vessel;
withdrawing the catheter slightly and transferring a proximal anchor from the tip of the catheter into the vessel;
longitudinally positioning the proximal anchor at a desired location;
expanding the proximal anchor such that a radially outer portion of the proximal anchor engages with an inner wall of the vessel, wherein an elastically deformable member extends longitudinally between the proximal and distal anchors.

Example 90. The method of example 89 wherein transferring the distal anchor includes advancing the distal anchor from the tip of the catheter.

Example 91. The method of example 89 or example 90 wherein transferring the distal anchor includes withdrawing the tip of the catheter whilst the distal anchor remains at a generally constant longitudinal position within the vessel, and exits from the tip of the catheter.

Example 92. The method of any one of examples 89-91 wherein longitudinally positioning the proximal anchor includes applying a first tensile force to one or more threads frangibly secured to the proximal anchor.

Example 93. The method of example 92, further including frangibly rupturing the thread(s) after expanding the proximal anchor by applying a second tensile force which is greater than the first tensile force.

Example 94. The method of example 92, further including disengaging a ring, latch or clasp secured to the thread(s) after expanding the proximal anchor in order to disengage the thread from the proximal anchor.

Example 95. The method of any one of examples 89-94, further including imaging to determine the location of the proximal and/or distal anchors.

Example 96. A method of treating a blood vessel selected from a left common carotid artery, a right common carotid artery or a brachiocephalic artery, a carotid artery, a branch of any of the foregoing, and an ascending aorta, the method comprising:
wrapping an elastically deformable material around the artery; and
attaching a first edge of the elastically deformable material to an opposing second edge of the elastically deformable material such that an internal diameter of the elastically deformable material is smaller than an initial outer diameter of the artery during a systole stage.

### J. Examples 97-113: Methods For Treating Dementia

Example 97. A method for treating dementia, comprising:
intravascularly positioning a damping device within an artery at a treatment site, wherein the damping device includes an anchoring member coupled to an elastic, tubular damping member defining a lumen therethrough;
expanding the anchoring member and the damping member from a low-profile state to an expanded state such that at least the anchoring member is in apposition with the arterial wall at the treatment site; and
changing a contour of the damping member in response to a pulse pressure wave in blood flow through the damping member.

Example 98. The method of example 97, further comprising reducing a magnitude of the pulse pressure transmitted to a portion of the blood vessel distal to the damping device.

Example 99. The method of example 98 wherein reducing a magnitude of the pulse pressure includes absorbing a portion of the pulsatile energy of blood flowing through the artery.

Example 100. The method of any one of examples 97-99 wherein changing a contour of the damping member includes increasing an inner diameter of the lumen damping member while an outer diameter of the damping member remains generally constant.

Example 101. The method of any one of examples 97-99 wherein changing a contour of the damping member includes increasing an inner diameter and an outer diameter of the lumen of the damping member.

Example 102. The method of any one of examples 97-99 wherein changing a contour of the damping member includes decreasing a distance between an inner surface of the damping member and an outer surface of the damping member.

Example 103. The method of example 97-102 wherein intravascularly positioning a damping device includes intravascularly positioning a damping device within a left common carotid artery at a treatment site.

Example 104. The method of any one of examples 97-103 wherein intravascularly positioning a damping device includes intravascularly positioning a damping device within a right common carotid artery at a treatment site.

Example 105. The method of any one of examples 97-104 wherein expanding the anchoring member and expanding the damping member occurs simultaneously.

Example 106. The method of any one of examples 97-105 wherein expanding the anchoring member includes expanding the anchoring member with a balloon.

Example 107. The method of any one of examples 97-105 wherein expanding the anchoring member includes withdrawing a sheath to expose the anchoring member to allow the anchoring member to self-expand.

Example 108. The method of any one of examples 97-107 wherein expanding the damping member includes expanding the damping member with a balloon.

Example 109. The method of any one of examples 97-107 wherein expanding the damping member includes withdrawing a sheath to expose the damping member to allow the anchoring member to self-expand.

Example 110. The method of any one of examples 97-109 wherein expanding the anchoring member forces the damping member to expand.

Example 111. The method of any one of examples 97-110 wherein:
the damping device is a first damping device,
the first damping device is intravascularly positioned at a first arterial location, and
the method further comprises intravascularly positioning a second damping device at a second arterial location different than the first arterial location.

Example 112. The method of example 111 wherein the first arterial location is one of a left common carotid artery, a right common carotid artery, an external carotid artery, an internal carotid artery, and an ascending aorta, and the second arterial location is one of a left common carotid artery, a right common carotid artery, an external carotid artery, an internal carotid artery, and an ascending aorta.

Example 113. The method of example 111 wherein the first arterial location is a left common carotid artery and the second arterial location is a right common carotid artery.

### K. Examples 114-115: Method for Treating And/Or Preventing the Effects of Dementia

Example 114. A method for treating and/or preventing the effects of dementia, comprising:
positioning a damping member along a length of an artery, the damping member including an abating substance; and
in response to a pulse wave traveling through blood in the artery, redistributing at least a portion of the abating compound along the length of the damping member, thereby attenuating at least a portion of the energy of the pulse wave in the blood.

Example 115. A method for treating and/or preventing the effects of dementia, comprising:
positioning a damping member along a length of an artery, the damping member including a plurality of fluid particles; and
moving a portion of the fluid particles away from an axial location along the damping member aligned a wavefront of a pulse wave, thereby increasing the inner diameter of the damping member.

### L. Examples 116-131: Devices for Treating And/Or Preventing the Progression Of Dementia

Example 116. A device for treating and/or preventing the progression of dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the arterial wall at the treatment site, thereby securing the damping member at the treatment site, and wherein the first and second anchoring members extend along only a portion of the length of the damping member such that at least a portion of the damping region is exposed between the first and second anchoring members and allowed to expand to a diameter greater than the deployed diameter.

Example 117. The device of example 116 wherein the damping member is elastically deformable, and is configured to deform in response to a change in blood/pulse pressure or elevated pulse wave intensity/FCWI.

Example 118. The device of example 116 or example 117 wherein, at a location along the damping member coincident with a leading end of a pulse pressure wave, the distance between the inner surface and the outer surface of the damping member decreases in response to the pressure.

Example 119. The device of any one of examples 116-118 wherein the lumen of the damping member has an hourglass shape.

Example 120. The device of any one of example 116-119 wherein the outer surface is generally cylindrical and the inner surface is undulating.

Example 121. The device of any one of examples 116-120 wherein each of the first and second anchoring members is an expandable stent.

Example 122. The device of any one of examples 116-120 wherein the each of the first and second anchoring members is an expandable mesh.

Example 123. The device of any one of examples 116-120 wherein each of the first and second anchoring members is at least one of an expandable stent and an expandable mesh.

Example 124. The device of any one of examples 116-123 wherein each of the first and second anchoring members is positioned around a circumference of the damping member.

Example 125. The device of any one of examples 116-124 wherein at least a portion of each of the first and second anchoring members is positioned within the damping member and extends through at least a portion of the thickness of the sidewall.

Example 126. The device of any one of examples 116-125 wherein the damping region is a first damping region, and wherein the damping member includes a plurality of damping regions between the first and second end portions.

Example 127. The device of any one of examples 116-126 wherein at least one of the first and second anchoring members comprise a plurality of fixation devices extending radially outwardly from the outer surface of the damping device.

Example 128. The device of any one of examples 116-127 wherein the device is configured to be positioned at a treatment site within the left common carotid artery.

Example 129. The device of any one of examples 116-127 wherein the device is configured to be positioned at a treatment site within the right common carotid artery.

Example 130. The device of any one of examples 116-129 wherein the device is configured to treat Alzheimer’s disease.

Example 131. The device of any one of examples 116-129 wherein the device is configured to reduce the occurrence of microbleeds in one or more branches of the artery downstream from the treatment site.

### M. Examples 132-147: Devices for Treating Dementia

Example 132. A device for treating dementia, comprising:
a damping member configured to be intravascularly positioned within an artery at a treatment site and having a lumen configured to direct blood flow to distal vasculature, the damping member being transformable between a low-profile state for delivery to the treatment site and an expanded state, wherein the damping member includes a damping region having a pressure limiter projecting laterally inwardly into the lumen to distribute pressure downstream from the damping member when a pulse pressure wave propagates along the damping member during systole; and
an anchoring member coupled to the damping member, wherein the anchoring member, in a deployed state, is configured to extend outwardly to a deployed diameter and contact a portion of the blood vessel wall at the treatment site, thereby securing the damping member at the treatment site, wherein the anchoring member extends along only a portion of the length of the damping member such that the damping region of the damping member is allowed to extend radially outward beyond the deployed diameter of the anchoring member.

Example 133. The device of example 132 wherein the damping member is elastically deformable, and is configured to deform in response to a change in blood/pulse pressure or elevated pulse wave intensity/FCWI.

Example 134. The device of example 132 or 133 wherein, at a location along the damping member coincident with a leading end of a pulse pressure wave, the distance between the inner surface and the outer surface of the damping member decreases in response to the pressure.

Example 135. The device of any one of examples 132-134 wherein the lumen of the damping member has an hourglass shape.

Example 136. The device of any one of examples 132-135 wherein the anchoring member is an expandable stent.

Example 137. The device of any one of examples 132-136 wherein the anchoring member is an expandable mesh.

Example 138. The device of any one of examples 132-137 wherein the anchoring member is at least one of an expandable stent and an expandable mesh.

Example 139. The device of any one of examples 132-138 wherein the anchoring member is positioned around a circumference of the damping member.

Example 140. The device of any one of examples 132-139 wherein at least a portion of the anchoring member is positioned within the damping member and extends through at least a portion of the thickness of the sidewall.

Example 141. The device of any one of examples 132-140 wherein the damping region is a first damping region, and wherein the damping member includes a plurality of damping regions between the first and second end portions.

Example 142. The device of any one of examples 132-141 wherein the anchoring member includes a plurality of fixation devices extending radially outwardly from the outer surface of the damping device.

Example 143. The device of any one of examples 132-142 wherein the device is configured to be positioned at a treatment site within the left common carotid artery.

Example 144. The device of any one of examples 132-142 wherein the device is configured to be positioned at a treatment site within the right common carotid artery.

Example 145. The device of any one of examples 132-144 wherein the device is configured to treat Alzheimer’s disease.

Example 146. The device of any one of examples 132-145 wherein the device is configured to reduce the occurrence of microbleeds in portions of the blood vessel downstream from the treatment site.

Example 147. A device for treating dementia, comprising:
a flexible, compliant damping member configured to be intravascularly positioned within an artery at a treatment site, the damping member being transformable between a low-profile state for delivery to the treatment site and an expanded state, wherein the damping member includes a generally tubular sidewall having (a) an outer surface, (b) an inner surface defining a lumen configured to direct blood flow, (c) a first end portion, (d) a second end portion opposite the first end portion along the length of the damping member, and (e) a damping region between the first and second end portions, wherein the inner surface and outer surface are spaced apart by a distance that is greater at the damping region than at either of the first or second end portions; and
a first anchoring member coupled to the first end portion of the damping member and a second anchoring member coupled to the second end portion of the damping member, wherein the first and second anchoring members, in a deployed state, extend radially to a deployed diameter configured to contact a portion of the blood vessel wall at the treatment site, thereby securing the damping member at the treatment site, and
wherein, when blood flows through the damping member during systole, the damping member absorbs a portion of the pulsatile energy of the blood, thereby reducing a magnitude of a pulse pressure transmitted to a portion of the blood vessel distal to the damping device.

### N. Examples 148-154: Devices for Treating a Blood Vessel

Example 148. A device for treating a blood vessel, comprising:
an anchoring system having a first portion and a second portion which is spaced apart from the first portion in a first direction; and
a cushioning member located between the first and second portions of the anchoring system such that movement of a portion of the cushioning member in a second direction, which is orthogonal to the first direction, is not constrained by the anchoring system, and wherein the cushioning member is configured to absorb pulsatile energy transmitted by blood flowing with the vessel.

Example 149. The device of example 148 wherein the cushioning member is elastically deformable and is configured to expand in response to an increase of blood/pulse pressure or elevated pulse wave intensity/FCWI within the vessel and relax as the blood/pulse pressure or elevated pulse wave intensity/FCWI within the vessel subsequently decreases.

Example 150. A device for treating a blood vessel, comprising:
an endovascular cushioning device having a proximal anchor and a distal anchor which is spaced apart from the proximal anchor, each of the proximal and distal anchors being configured to abut against an inner wall of a major artery; and
an elastically deformable member extending between the proximal and distal anchors,
wherein the elastically deformable member is configured to expand in response to an increase of blood/pulse pressure or elevated pulse wave intensity/FCWI within the vessel and relax as the blood/pulse pressure or elevated pulse wave intensity/FCWI within the vessel subsequently decreases.

Example 151. The device of example 150 wherein a portion of the elastically deformable membrane located longitudinally between the proximal and distal anchors defines a region of reduced internal cross-sectional area relative to the proximal and distal anchors when the elastically deformable membrane is radially relaxed.

Example 152. The device of example 150 or example 151 wherein the proximal and distal anchors are each radially expandable between a first diameter before deployment and a second diameter after deployment.

Example 153. The device of any one of examples 150-152, further comprising one or more threads secured to the proximal anchor.

Example 154. The device of example 153 wherein each thread is secured to an eyelet.

### O. Examples 155-160: Devices for Treating an Artery Selected From a Left Common Carotid Artery, a Right Common Carotid Artery, a Brachiocephalic Artery, The Ascending Aorta, an Internal Carotid Artery, Or An Abdominal Aorta

Example 155. A device for treating an artery selected from a left common carotid artery, a right common carotid artery, a brachiocephalic artery, the ascending aorta, an internal carotid artery, or an abdominal aorta, the device comprising:
a wrap fabricated from an elastically deformable material, and
an engagement formation adapted to secure two opposing edges of the wrap around the artery,
wherein the elastically deformable material is configured to radially expand during a systole stage and radially contract during a diastole stage.

Example 156. The device of example 155 wherein, when the wrap is in position around the artery, the wrap entirely or substantially entirely surrounds the artery over a portion of its length.

Example 157. The device of example 155 wherein the engagement formation includes sutures and/or staples.

Example 158. The device of example 155 wherein the engagement formation includes a zip lock.

Example 159. A device for treating a left common carotid artery, a right common carotid artery, a brachiocephalic artery, or an ascending aorta, the device comprising:
a proximal anchor configured to be wrapped around the artery;
a distal anchor configured to be wrapped around the artery and longitudinally spaced relative to the proximal anchor; and
a helical band adapted to be wound around the artery, the helical band having a first end securable to the proximal anchor and an opposing second end securable to the distal anchor, wherein the helical band is adapted to radially expand during a systole stage and radially contract during a diastole stage.

Example 160. The device of example 159 wherein the first end of the helical band is secured to the proximal anchor and the second end of the helical band is secured to the distal anchor.

### P. Examples 161-214: Devices for Treating And/or Preventing the Effects Of Dementia

Example 161. A device for treating and/or preventing the effects of dementia, comprising:

a damping member comprising a deformable, generally tubular sidewall having an outer surface and an inner surface that is undulating in a longitudinal direction, and wherein the sidewall is configured to be positioned in apposition with a blood vessel wall to absorb pulsatile energy transmitted by blood flowing through the blood vessel.

Example 162. The device of example 161 wherein the damping member is configured to be positioned in apposition with at least one of a left common carotid artery, a right common carotid artery, and a brachiocephalic artery.

Example 163. The device of example 161 wherein the damping member is configured to be positioned in apposition with an ascending aorta.

Example 164. The device of any one of examples 161-163 wherein the damping member is configured to be positioned in apposition with an inner surface of the blood vessel wall.

Example 165. The device of any one of examples 161-163 wherein the damping member is configured to be positioned in apposition with an outer surface of the blood vessel wall.

Example 166. The device of any one of examples 161-165 wherein the sidewall has an inner diameter, and, when the damping member is in a deployed state, the inner diameter increases then decreases in an axial direction.

Example 167. The device of any one of examples 161-166 wherein the cross-sectional area decreases then increases in longitudinal direction.

Example 168. The device of any one of examples 161-167 wherein the outer surface has a generally cylindrical shape.

Example 169. The device of any one of examples 161-167 wherein the outer surface has an undulating shape.

Example 170. The device of any one of examples 161-169, further comprising an anchoring member coupled to the damping member and axially aligned with only a portion of the damping member, wherein the anchoring member is configured to engage the blood vessel wall and secure the damping member to the blood vessel wall.

Example 171. The device of example 170 wherein the anchoring member is a first anchoring member and the device further comprises a second anchoring member coupled to the damping member, and wherein the second anchoring member:
is axially aligned with only a portion of the damping member, and
is spaced apart from the first anchoring member along the longitudinal axis of the damping member.

Example 172. The device of any one of examples 161-171 wherein, when the damping member is positioned adjacent the blood vessel wall, the damping member does not constrain the diameter of the blood vessel wall.

Example 173. A device for treating and/or preventing the effects of dementia, comprising:

an elastic member which is configured to abut an arterial wall and form a generally tubular structure having an inner diameter, an outer diameter, an outer surface, and an undulating inner surface, and wherein at least one of the outer diameter and the inner diameter increases and decreases in response to an increase and a decrease in pulse pressure within the blood vessel, respectively.

Example 174. The device of example 173 wherein the elastic member is configured to be positioned in apposition with at least one of a left common carotid artery, a right common carotid artery, and a brachiocephalic artery.

Example 175. The device of example 173 wherein the elastic member is configured to be positioned in apposition with an ascending aorta.

Example 176. The device of any one of examples 173-175 wherein the elastic member is configured to be positioned in apposition with an inner surface of the blood vessel wall.

Example 177. The device of any one of examples 173-175 wherein the elastic member is configured to be positioned in apposition with an outer surface of the blood vessel wall.

Example 178. The device of any one of examples 173-177 wherein the sidewall has an inner diameter, and, when the elastic member is in a deployed state, the inner diameter increases then decreases in an axial direction.

Example 179. The device of any one of examples 173-178 wherein the cross-sectional area decreases then increases in longitudinal direction.

Example 180. The device of any one of examples 173-179 wherein the outer surface has a generally cylindrical shape.

Example 181. The device of any one of examples 173-179 wherein the outer surface has an undulating shape.

Example 182. The device of any one of examples 173-181, further comprising an anchoring member coupled to the elastic member and axially aligned with only a portion of the elastic member, wherein the anchoring member is configured to engage the blood vessel wall and secure the elastic member to the blood vessel wall.

Example 183. The device of example 182 wherein the anchoring member is a first anchoring member and the device further comprises a second anchoring member coupled to the elastic member, and wherein the second anchoring member:
is axially aligned with only a portion of the elastic member, and
is spaced apart from the first anchoring member along the longitudinal axis of the elastic member.

Example 184. The device of any one of examples 173 to 183 wherein, when the elastic member is positioned adjacent the blood vessel wall, the elastic member does not constrain the diameter of the blood vessel wall.

Example 185. The device of any one of examples 173 to 184 wherein the damping member or elastic member has a low-profile state and a deployed state.

Example 186. The device of example 185 wherein the deployed state is for delivery to a treatment site at a blood vessel wall.

Example 187. The device of example 185 or 186 wherein the damping member or elastic member has a first, lesser outer diameter when in the low-profile state and a second, greater diameter when in the deployed state.

Example 188. A device for treating and/or preventing the effects of dementia, comprising:

a damping member including an abating substance, wherein the damping member forms a generally tubular structure having an axis, wherein the abating substance is able to move axially relative to the tubular structure, and wherein the damping member is configured to be positioned along the circumference of an artery such that, when a pulse wave traveling through the artery applies a stress at a first axial location along the length of the tubular structure, at least a portion of the abating substance moves away from the first location to a second axial location along the length of the tubular structure.

Example 189. The device of example 188, wherein the abating substance comprises a quantity of a fluid and/or gel comprising particles, contained within a flexible member, and the particles may move axially relative to the tubular structure within the flexible member.

Example 190. The device of example 189 wherein the flexible member may, at least some locations along the length of the tubular structure, be deformed radially with respect to the tubular structure.

Example 191. The device of any one of examples 188-190, further comprising a structural element coupled to the damping member.

Example 192. The device of any one of examples 188-191 wherein, in a deployed state, the damping member is configured to wrap around at least a portion of the circumference of the artery.

Example 193. The device of example 192 wherein the damping member includes a break along its length, to allow it to be fitted around the portion of the circumference of the artery.

Example 194. The device of example 193, further comprising cooperating sealing arrangements located on or near opposing edges of the break, to allow the edges to be joined together once the damping member has been fitted around the portion of the circumference of the artery.

Example 195. The device of any one of examples 188-194 wherein, in a deployed state, the device has a pre-set helical configuration.

Example 196. The device of any one of examples 188-195 wherein the damping member includes a liquid.

Example 197. The device of any one of examples 188-196 wherein the damping member includes a gas.

Example 198. The device of any one of examples 188-197 wherein the damping member includes a gel.

Example 199. The device of any one of examples 188-198 wherein the damping member, in a deployed configuration, is configured to be positioned in apposition with an outer surface of the arterial wall.

Example 200. The device of any one of examples 188-199 wherein the damping member, in a deployed configuration, is configured to be positioned around the arterial wall such that an inner surface of the damping member is in contact with blood flowing through the artery.

Example 201. A device for treating and/or preventing the effects of dementia, comprising:
wherein the fluid particles are able to move axially along at least a part of the length of the damping structure, the damping member being configured to be positioned along the circumference of an artery at a treatment site along a length of the artery,
wherein, when the damping member is in a deployed configuration and positioned at the treatment site, a wave front traveling through the length of the artery redistributes at least a portion of the fluid particles along the length of the damping member such that the inner diameter of the damping member increases at the axial location along the damping member aligned with the wave front while the inner diameter of the damping member at another axial location along the damping member decreases.

Example 202. The device of example 201 wherein the fluid particles are contained within a flexible member, and the particles may move along the length of the damping member within the flexible member.

Example 203. The device of example 202 wherein the flexible member may, at least some locations along the length of the damping member, be deformed radially with respect to the damping member.

Example 204. The device of any one of examples 201-203, further comprising a structural element coupled to the damping member.

Example 205. The device of any one of examples 201-204 wherein, in the deployed state, the damping member is configured to wrap around at least a portion of the circumference of the artery.

Example 206. The device of example 205 wherein the damping member includes a break along its length, to allow it to be fitted around the portion of the circumference of the artery.

Example 207. The device of example 206, further comprising cooperating sealing arrangements located on or near opposing edges of the break, to allow the edges to be joined together once the damping member has been fitted around the portion of the circumference of the artery.

Example 208. The device of any one of examples 201-207 wherein, in the deployed state, the device has a pre-set helical configuration.

Example 209. The device of any one of examples 201-208 wherein the damping member includes a liquid.

Example 210. The device of any one of examples 201-209 wherein the damping member includes a gas.

Example 211. The device of any one of examples 201-210 wherein the damping member includes a gel.

Example 212. The device of any one of examples 201-211 wherein the damping member, in the deployed configuration, is configured to be positioned in apposition with an outer surface of the arterial wall.

Example 213. The device of any one of examples 201-212 wherein the damping member, in the deployed configuration, is configured to be positioned around the arterial wall such that an inner surface of the damping member is in contact with blood flowing through the artery.

Example 214. The device of any one of examples 201-213 wherein the damping member has a low-profile configuration and a deployed configuration.

### Q. Examples 215-401: Methods for Treating And/Or Preventing One or More Effects Of a Condition

Example 215. A method for treating and/or preventing one or more effects of a condition in a subject in need thereof, the method comprising:
providing a device for treating and/or preventing one or more effects of the condition, and configured to be placed in apposition with a blood vessel, the device comprising —
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel; and
providing a biologic therapy that treats or slows one or more effects of the condition in combination with the device.

Example 216. The method of example 215, wherein the biologic therapy is a cell-based therapy, a gene-based therapy, a microbiome-based therapy, and/or a peptide-based therapy.

Example 217. The method of example 216, wherein the cell-based therapy is stem cell therapy or progenitor cell therapy.

Example 218. The method of example 217, wherein the stem cell therapy is allogenic or autologous human mesenchymal stem cell therapy.

Example 219. The method of example 217, wherein the stem cell therapy is autologous bone-marrow derived or adipose-derived human mesenchymal stem cell therapy.

Example 220. The method of example 217, wherein the stem cell therapy is autologous umbilical cord blood-derived or placenta-derived human mesenchymal stem cell therapy.

Example 221. The method of example 216, wherein the gene-based therapy includes at least one viral vector.

Example 222. The method of example 221, wherein the at least one viral vector is an adeno-associated viral (AAV) vector.

Example 223. The method of example 222, wherein the AAV vector includes a nucleic acid that reduces or prevents expression of APOE4.

Example 224. The method of example 222, wherein the AAV vector includes a nucleic acid that encodes NGF or hTERT.

Example 225. The method of example 216, wherein the microbiome-based therapy includes providing one or more fecal microbiota transplant therapeutic agents to the subj ect.

Example 226. The method of example 225, wherein the fecal microbiota transplant therapeutic agents are capsules containing at least one microbiota organism derived or isolated from feces.

Example 227. The method of example 216, wherein the microbiome-based therapy includes providing a probiotic to the subject.

Example 228. The method of example 227, wherein the probiotic is Omni-Biotic stress repair.

Example 229. The method of example 216, wherein the microbiome-based therapy includes providing a sodium oligo-mannurarate remodelling agent to the subject.

Example 230. The method of example 229, wherein the sodium oligo-mannurarate remodelling agent is GV-971.

Example 231. The method of example 216, wherein the peptide-based therapy includes an ETB receptor agonist (e.g., IRL-1620) or a peptide derivative or a variant of activated protein kinase C (APC).

Example 232. The method of example 231, wherein the peptide derivative or variant of APC includes an amino acid sequence which differs from an amino acid sequence of wild-type APC.

Example 233. The method of example 231 or example 232, wherein the peptide derivative or variant of APC is 3K3A-APC.

Example 234. The method of any one of examples 215 to 233, wherein the biologic therapy prevents and/or reduces expression of APOE4 or increases the expression of APOE2 or APOE3, promotes expression of one or more neurotrophic factors, prevents and/or reduces an abnormal biome from developing in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents dysregulation/damage and/or death of a neuron, prevent dysregulation and/or damage to the blood brain barrier, and/or reduces and/or prevents insulin resistance.

Example 235. The method of any one of examples 215 to 234, wherein the biologic therapy is provided at a first dosage which is lower than a second dosage that is provided in the absence of the device.

Example 236. The method of any one of examples 215 to 235, wherein the biologic therapy is provided at a first dosing regimen which is less than a second dosing regimen that is provided in the absence of the device.

Example 237. The method of any one of examples 215 to 236, wherein the biologic therapy is provided via a first route which is different than a second route that is provided in the absence of the device.

Example 238. The method of any one of examples 215 to 235, wherein the biologic therapy is provided by administering the biologic therapy to the subject in need thereof.

Example 239. The method of any one of examples 215 to 236, wherein the condition is neurodegeneration.

Example 240. The method of example 239, wherein neurodegeneration further comprises Alzheimer’s disease, dementia, and/or cognitive impairment.

Example 241. The method of any one of examples 215 to 240, wherein the device has a low-profile state and a deployed state, and when in the deployed state, the sidewall is generally tubular.

Example 242. The method of any one of examples 215 to 241, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 243. The method of example 242, wherein when positioned in apposition with the blood vessel and a pulse wave travels through the blood vessel, the flexible damping member applies a stress at the first location along a length of the tubular structure.

Example 244. The method of example 243, wherein, after stress is applied at the first location, at least the portion of the abating substance moves longitudinally and/or radially along a length of the tubular structure.

Example 245. The method of example 243, wherein, after stress is applied at the first location, at least a portion of the abating substance is configured to move longitudinally and/or radially from a first location within a first deformable portion to a second location within the first deformable portion of the flexible damping member.

Example 246. The method of example 243, wherein, after stress is applied at the first location, at least the portion of the abating substance is further configured to move longitudinally and/or radially from the first location to a third location within a second deformable portion of the flexible damping member.

Example 247. The method of any one of examples 215 to 246, wherein the inner surface and/or an outer surface has a generally cylindrical shape or an undulating shape that undulates in a longitudinal direction.

Example 248. The method of any one of examples 215 to 247, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 249. The method of any one of examples 215 to 248, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 250. The method of example 249, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 251. A method for treating and/or preventing one or more effects of a condition in a subject in need thereof, the method comprising:
providing a device for treating and/or preventing one or more effects of the condition and configured to be placed in apposition with a blood vessel, the device comprising
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel; and
wherein a biologic therapy that treats or slows one or more effects of the condition has previously been provided to the subject in need thereof.

Example 252. The method of example 251, wherein the biologic therapy is a cell-based therapy, a gene-based therapy, a microbiome-based therapy, and/or a peptide-based therapy.

Example 253. The method of example 252, wherein the cell-based therapy is stem cell therapy or progenitor cell therapy.

Example 254. The method of example 253, wherein the stem cell therapy is allogenic or autologous human mesenchymal stem cell therapy.

Example 255. The method of example 253, wherein the stem cell therapy is autologous bone-marrow derived or adipose-derived human mesenchymal stem cell therapy.

Example 256. The method of example 253, wherein the stem cell therapy is autologous umbilical cord blood-derived or placenta-derived human mesenchymal stem cell therapy.

Example 257. The method of example 252, wherein the gene-based therapy includes at least one viral vector.

Example 258. The method of example 257, wherein the at least one viral vector is an adeno-associated viral (AAV) vector.

Example 259. The method of example 258, wherein the AAV vector includes a nucleic acid that reduces or prevents expression of APOE4.

Example 260. The method of example 258, wherein the AAV vector includes a nucleic acid that encodes NGF or hTERT.

Example 261. The method of example 252, wherein the microbiome-based therapy includes providing one or more fecal microbiota transplant therapeutic agents to the subj ect.

Example 262. The method of example 261, wherein the fecal microbiota transplant therapeutic agents are capsules containing at least one microbiota organism derived or isolated from feces.

Example 263. The method of example 252, wherein the microbiome-based therapy includes providing a probiotic to the subject.

Example 264. The method of example 263, wherein the probiotic is Omni-Biotic stress repair.

Example 265. The method of example 252, wherein the microbiome-based therapy includes providing a sodium oligo-mannurarate remodelling agent to the subject.

Example 266. The method of example 265, wherein the sodium oligo-mannurarate remodelling agent is GV-971.

Example 267. The method of example 252, wherein the peptide-based therapy includes an ETB receptor agonist (e.g., IRL-1620) or a peptide derivative or variant of activated protein kinase C (APC).

Example 268. The method of example 267, wherein the peptide derivative or variant of APC includes an amino acid sequence which differs from an amino acid sequence of wild-type APC.

Example 269. The method of example 267 or example 268, wherein the peptide derivative or variant of APC is 3K3A-APC.

Example 270. The method of any one of examples 251 to 269, wherein the biologic therapy prevents and/or reduces expression of APOE4 or increases the expression of APOE2 or APOE3, promotes expression of one or more neurotrophic factors, prevents and/or reduces an abnormal biome in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, and/or reduces and/or prevents insulin resistance.

Example 271. The method of any one of examples 251 to 270, wherein the biologic therapy is provided at a first dosage which is lower than a second dosage that is provided in the absence of the device.

Example 272. The method of any one of examples 251 to 271, wherein the biologic therapy is provided at a first dosing regimen which is less than a second dosing regimen that is provided in the absence of the device.

Example 273. The method of any one of examples 251 to 272, wherein the biologic therapy is provided via a first route which is different than a second route that is provided in the absence of the device.

Example 274. The method of any one of examples 251 to 273, wherein the biologic therapy is provided by administering the biologic therapy to the subject in need thereof.

Example 275. The method of any one of examples 251 to 274, wherein the condition is neurodegeneration.

Example 276. The method of example 275, wherein neurodegeneration further comprises Alzheimer’s disease, dementia, and/or cognitive impairment.

Example 277. The method of any one of examples 251 to 276, wherein the device has a low-profile state and a deployed state, and when in the deployed state, the sidewall is generally tubular.

Example 278. The method of any one of examples 251 to 277, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 279. The method of example 278, wherein when positioned in apposition with the blood vessel and a pulse wave travels through the blood vessel, the flexible damping member applies a stress at the first location along a length of the tubular structure.

Example 280. The method of example 279, wherein, after stress is applied at the first location, at least the portion of the abating substance moves longitudinally and/or radially along a length of the tubular structure.

Example 281. The method of example 279, wherein, after stress is applied at the first location, at least a portion of the abating substance is configured to move longitudinally and/or radially from a first location within a first deformable portion to a second location within the first deformable portion of the flexible damping member.

Example 282. The method of example 279, wherein, after stress is applied at the first location, at least the portion of the abating substance is further configured to move longitudinally and/or radially from the first location to a third location within a second deformable portion of the flexible damping member.

Example 283. The method of any one of examples 251 to 282, wherein the inner surface and/or an outer surface has a generally cylindrical shape or an undulating shape that undulates in a longitudinal direction.

Example 284. The method of any one of examples 251 to 283, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 285. The method of any one of examples 251 to 284, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 286. The method of example 285, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 287. A method for treating and/or preventing one or more effects of a condition in a subject in need thereof, the method comprising:
providing a biologic therapy for treating and/or preventing one or more effects of the condition to the subject in need thereof, wherein the subject has previously been provided a device that treats or slows one or more effects of the condition and was placed in apposition with a blood vessel, the device comprising —
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel.

Example 288. The method of example 287, wherein the biologic therapy is a cell-based therapy, a gene-based therapy, a microbiome-based therapy, and/or a peptide-based therapy.

Example 289. The method of example 288, wherein the cell-based therapy is stem cell therapy or progenitor cell therapy.

Example 290. The method of example 289, wherein the stem cell therapy is allogenic or autologous human mesenchymal stem cell therapy.

Example 291. The method of example 289, wherein the stem cell therapy is autologous bone-marrow derived or adipose-derived human mesenchymal stem cell therapy.

Example 292. The method of example 289, wherein the stem cell therapy is autologous umbilical cord blood-derived or placenta-derived human mesenchymal stem cell therapy.

Example 293. The method of example 288, wherein the gene-based therapy includes at least one viral vector.

Example 294. The method of example 293, wherein the at least one viral vector is an adeno-associated viral (AAV) vector.

Example 295. The method of example 294, wherein the AAV vector includes a nucleic acid that reduces or prevents expression of APOE4.

Example 296. The method of example 294, wherein the AAV vector includes a nucleic acid that encodes NGF or hTERT.

Example 297. The method of example 288, wherein the microbiome-based therapy includes providing one or more fecal microbiota transplant therapeutic agents to the subj ect.

Example 298. The method of example 297, wherein the fecal microbiota transplant therapeutic agents are capsules containing at least one microbiota organism derived or isolated from feces.

Example 299. The method of example 288, wherein the microbiome-based therapy includes providing a probiotic to the subject.

Example 300. The method of example 299, wherein the probiotic is Omni-Biotic stress repair.

Example 301. The method of example 288, wherein the microbiome-based therapy includes providing a sodium oligo-mannurarate remodelling agent to the subject.

Example 302. The method of example 301 wherein the sodium oligo-mannurarate remodelling agent is GV-971.

Example 303. The method of example 287, wherein the peptide-based therapy includes an ETB receptor agonist (e.g., IRL-1620) or a peptide derivative or variant of activated protein kinase C (APC).

Example 304. The method of example 303, wherein peptide derivative or variant of APC includes an amino acid sequence which differs from an amino acid sequence of wild-type APC.

Example 305. The method of example 303 or example 304, wherein the peptide derivative or variant of APC is 3K3A-APC.

Example 306. The method of any one of examples 287 to 305, wherein the biologic therapy prevents and/or reduces expression of APOE4 or increases the expression of APOE2 or APOE3, promotes expression of one or more neurotrophic factors, prevents and/or reduces an abnormal biome in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, and/or reduces and/or prevents insulin resistance.

Example 307. The method of any one of examples 287 to 306, wherein the biologic therapy is provided at a first dosage which is lower than a second dosage that is provided in the absence of the device.

Example 308. The method of any one of examples 287 to 307, wherein the biologic therapy is provided at a first dosing regimen which is less than a second dosing regimen that is provided in the absence of the device.

Example 309. The method of any one of examples 287 to 308, wherein the biologic therapy is provided via a first route which is different than a second route that is provided in the absence of the device.

Example 310. The method of any one of examples 287 to 309, wherein the biologic therapy is provided by administering the biologic therapy to the subject in need thereof.

Example 311. The method of any one of examples 287 to 310, wherein the condition is neurodegeneration.

Example 312. The method of example 311, wherein neurodegeneration further comprises Alzheimer’s disease, dementia, and/or cognitive impairment.

Example 313. The method of any one of examples 287 to 312, wherein the device has a low-profile state and a deployed state, and when in the deployed state, the sidewall is generally tubular.

Example 314. The method of any one of examples 287 to 313, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 315. The method of example 314, wherein when positioned in apposition with the blood vessel and a pulse wave travels through the blood vessel, the flexible damping member applies a stress at the first location along a length of the tubular structure.

Example 316. The method of example 315, wherein, after stress is applied at the first location, at least the portion of the abating substance moves longitudinally and/or radially along a length of the tubular structure.

Example 317. The method of example 315, wherein, after stress is applied at the first location, at least a portion of the abating substance is configured to move longitudinally and/or radially from a first location within a first deformable portion to a second location within the first deformable portion of the flexible damping member.

Example 318. The method of example 315, wherein, after stress is applied at the first location, at least the portion of the abating substance is further configured to move longitudinally and/or radially from the first location to a third location within a second deformable portion of the flexible damping member.

Example 319. The method of any one of examples 287 to 318, wherein the inner surface and/or an outer surface has a generally cylindrical shape or an undulating shape that undulates in a longitudinal direction.

Example 320. The method of any one of examples 287 to 319, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 321. The method of any one of examples 287 to 320, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 322. The method of example 321, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 323. A system for treating and/or preventing one or more effects of a condition in a subject in need thereof, the system comprising:
an effective amount of a biologic therapy for treating and/or preventing one or more effects of the condition
a device for treating and/or preventing one or more effects of the condition,
the device comprising —
   a flexible damping member forming a generally tubular structure having an inner surface and an outer surface, the inner surface formed of a sidewall having one or more at least partially deformable portions, and
   an abating substance disposed within the one or more at least partially deformable portions of the sidewall configured to move longitudinally and/or radially within one partially deformable portion in response to pulsatile blood flow within the blood vessel.

Example 324. The system of example 323, wherein the biologic therapy is a cell-based therapy, a gene-based therapy, a microbiome-based therapy, and/or a peptide-based therapy.

Example 325. The system of example 324, wherein the cell-based therapy is stem cell therapy or progenitor cell therapy.

Example 326. The system of example 325, wherein the stem cell therapy is allogenic or autologous human mesenchymal stem cell therapy.

Example 327. The system of example 325, wherein the stem cell therapy is autologous bone-marrow derived or adipose-derived human mesenchymal stem cell therapy.

Example 328. The system of example 325, wherein the stem cell therapy is autologous umbilical cord blood-derived or placenta-derived human mesenchymal stem cell therapy.

Example 329. The system of example 324, wherein the gene-based therapy includes at least one viral vector.

Example 330. The system of example 329, wherein the at least one viral vector is an adeno-associated viral (AAV) vector.

Example 331. The system of example 330, wherein the AAV vector includes a nucleic acid that reduces or prevents expression of APOE4.

Example 332. The system of example 331, wherein the AAV vector includes a nucleic acid that encodes NGF or hTERT.

Example 333. The system of example 324, wherein the microbiome-based therapy includes providing one or more fecal microbiota transplant therapeutic agents to the subj ect.

Example 334. The system of example 333, wherein the fecal microbiota transplant therapeutic agents are capsules containing at least one microbiota organism derived or isolated from feces.

Example 335. The system of example 324, wherein the microbiome-based therapy includes providing a probiotic to the subject.

Example 336. The system of example 335, wherein the probiotic is Omni-Biotic stress repair.

Example 337. The system of example 324, wherein the microbiome-based therapy includes providing a sodium oligo-mannurarate remodelling agent to the subject.

Example 338. The system of example 337 wherein the sodium oligo-mannurarate remodelling agent is GV-971.

Example 339. The method of example 323, wherein the peptide-based therapy includes an ETB receptor agonist (e.g., IRL-1620) or a peptide derivative or variant of activated protein kinase C (APC).

Example 340. The method of example 339, wherein the peptide derivative or variant of APC includes an amino acid sequence which differs from an amino acid sequence of wild-type APC.

Example 341. The method of example 339 or example 340, wherein the peptide derivative or variant of APC is 3K3A-APC.

Example 342. The method of any one of examples 323 to 341, wherein the biologic therapy prevents and/or reduces expression of APOE4 or increases the expression of APOE2 or APOE3, promotes expression of one or more neurotrophic factors, prevents and/or reduces an abnormal biome from developing in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, and/or reduces and/or prevents insulin resistance.

Example 343. The system of any one of examples 323 to 342, wherein the biologic therapy is provided at a first dosage which is lower than a second dosage that is provided in the absence of the device.

Example 344. The system of any one of examples 323 to 343, wherein the biologic therapy is provided at a first dosing regimen which is less than a second dosing regimen that is provided in the absence of the device.

Example 345. The system of any one of examples 323 to 344, wherein the biologic therapy is provided via a first route which is different than a second route that is provided in the absence of the device.

Example 346. The system of any one of examples 323 to 345, wherein the biologic therapy is provided by administering the biologic therapy to the subject in need thereof.

Example 347. The system of any one of examples 323 to 346, wherein the condition is neurodegeneration.

Example 348. The system of example 347, wherein neurodegeneration further comprises Alzheimer’s disease, dementia, and/or cognitive impairment.

Example 349. The system of any one of examples 323 to 348, wherein the inner surface and/or an outer surface has a generally cylindrical shape or an undulating shape that undulates in a longitudinal direction.

Example 350. The system of any one of examples 323 to 349, wherein the device has a low-profile state and a deployed state, and when in the deployed state, the sidewall is generally tubular.

Example 351. The system of any one of examples 323 to 350, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 352. The system of example 351, wherein when positioned in apposition with the blood vessel and a pulse wave travels through the blood vessel, the flexible damping member applies a stress at the first location along a length of the tubular structure.

Example 353. The system of example 352, wherein, after stress is applied at the first location, at least the portion of the abating substance moves longitudinally and/or radially along a length of the tubular structure.

Example 354. The system of example 352, wherein, after stress is applied at the first location, at least a portion of the abating substance is configured to move longitudinally and/or radially from a first location within a first deformable portion to a second location within the first deformable portion of the flexible damping member.

Example 355. The system of example 352, wherein, after stress is applied at the first location, at least the portion of the abating substance is further configured to move longitudinally and/or radially from the first location to a third location within a second deformable portion of the flexible damping member.

Example 356. The system of any one of examples 323 to 355, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 357. The system of any one of examples 323 to 356, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 358. The system of example 357, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

Example 359. A system for treating and/or preventing one or more effects of a condition in a subject in need thereof, the system comprising:
an effective amount of a biologic therapy for treating and/or preventing one or more effects of the condition
a device for treating and/or preventing one or more effects of the condition,
the device comprising —
   a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
   an abating substance disposed within the one or more at least partially deformable portions of the sidewall configured to move longitudinally and/or radially within one partially deformable portion in response to pulsatile blood flow within the blood vessel;
wherein, the effective amount of the biologic therapy for treating and/or preventing one or more effects of the condition is carried by biologic or more of the at least partially deformable portions of the device, and
wherein, when the one or more at least partially deformable portions are at least partially deformed, the effective amount of biologic therapy for treating and/or preventing one or more effects of the condition is released from the device.

Example 360. The system of example 359, wherein the effective amount of the biologic therapy further comprises a first effective amount of the biologic therapy and a second effective amount of the biologic therapy.

Example 361. The system of example 360, wherein the second effective amount of the biologic therapy is greater than the first effective amount of the biologic therapy.

Example 362. The system of example 360, wherein, in response to a first pulsatile blood flow within the blood vessel, the one or more at least partially deformable portions are at least partially deformed to a first degree of deformation.

Example 363. The system of example 362, wherein, in response to a second pulsatile blood flow within the blood vessel, the one or more at least partially deformable portions are at least partially deformed to a second degree of deformation.

Example 364. The system of example 363, wherein the second degree of deformation is greater than the first degree of deformation.

Example 365. The system of example 364, wherein the first effective amount of the biologic therapy is released from the one or more at least partially deformable portions in response to the first degree of deformation.

Example 366. The system of example 365, wherein the second effective amount of the biologic therapy is released from the one or more at least partially deformable portions in response to the second degree of deformation.

Example 367. The system of any one of examples 359 to 366, wherein the biologic therapy is a cell-based therapy, a gene-based therapy, a microbiome-based therapy, and/or a peptide-based therapy.

Example 368. The system of example 367, wherein the cell-based therapy is stem cell therapy or progenitor cell therapy.

Example 369. The system of example 368, wherein the stem cell therapy is allogenic or autologous human mesenchymal stem cell therapy.

Example 370. The system of example 368, wherein the stem cell therapy is autologous bone-marrow derived or adipose-derived human mesenchymal stem cell therapy.

Example 371. The system of example 368, wherein the stem cell therapy is autologous umbilical cord blood-derived or placenta-derived human mesenchymal stem cell therapy.

Example 372. The system of example 367, wherein the gene-based therapy includes at least one viral vector.

Example 373. The system of example 372, wherein at least one viral vector is an adeno-associated viral (AAV) vector.

Example 374. The system of example 373, wherein the AAV vector includes a nucleic acid that reduces or prevents expression of APOE4.

Example 375. The system of example 373, wherein the AAV vector includes a nucleic acid that encodes NGF or hTERT.

Example 376. The system of example 367, wherein the microbiome-based therapy includes providing one or more fecal microbiota transplant therapeutic agents to the subj ect.

Example 377. The system of example 376, wherein the fecal microbiota transplant therapeutic agents are capsules containing at least one microbiota organism derived or isolated from feces.

Example 378. The system of example 367, wherein the microbiome-based therapy includes providing a probiotic to the subject.

Example 379. The system of example 378, wherein the probiotic is Omni-Biotic stress repair.

Example 380. The system of example 367, wherein the microbiome-based therapy includes providing a sodium oligo-mannurarate remodelling agent to the subject.

Example 381. The system of example 380 wherein the sodium oligo-mannurarate remodelling agent is GV-971.

Example 382. The method of example 359, wherein the peptide-based therapy includes an ETB receptor agonist (e.g., IRL-1620) or a peptide derivative or variant of activated protein kinase C (APC).

Example 383. The method of example 382, wherein the peptide derivative or variant of APC includes an amino acid sequence which differs from an amino acid sequence of wild-type APC.

Example 384. The method of example 382 or example 383, wherein the peptide derivative or variant of APC is 3K3A-APC.

Example 385. The method of any one of examples 359 to 384, wherein the biologic therapy prevents and/or reduces expression of APOE4 or increases the expression of APOE2 or APOE3, promotes expression of one or more neurotrophic factors, prevents and/or reduces an abnormal biome from developing in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, and/or reduces and/or prevents insulin resistance.

Example 386. The system of any one of examples 359 to 385, wherein the therapy is provided at a first dosage which is lower than a second dosage that is provided in the absence of the device.

Example 387. The system of any one of examples 359 to 386, wherein the therapy is provided at a first dosing regimen which is less than a second dosing regimen that is provided in the absence of the device.

Example 388. The system of any one of examples 359 to 387, wherein the therapy is provided via a first route which is different than a second route that is provided in the absence of the device.

Example 389. The system of any one of examples 359 to 388, wherein the therapy is provided by administering the therapy to the subject in need thereof.

Example 390. The system of any one of examples 359 to 389, wherein the condition is neurodegeneration.

Example 391. The system of example 390, wherein neurodegeneration further comprises Alzheimer’s disease, dementia, and/or cognitive impairment.

Example 392. The system of any one of examples 359 to 391, wherein the inner surface and/or an outer surface has a generally cylindrical shape or an undulating shape that undulates in a longitudinal direction.

Example 393. The system of any one of examples 359 to 392, wherein the device has a low-profile state and a deployed state, and when in the deployed state, the sidewall is generally tubular.

Example 394. The system of any one of examples 359 to 393, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 395. The system of example 394, wherein when positioned in apposition with the blood vessel and a pulse wave travels through the blood vessel, the flexible damping member applies a stress at the first location along a length of the tubular structure.

Example 396. The system of example 395 wherein, after stress is applied at the first location, at least the portion of the abating substance moves longitudinally and/or radially along a length of the tubular structure.

Example 397. The system of example 395, wherein, after stress is applied at the first location, at least a portion of the abating substance is configured to move longitudinally and/or radially from a first location within a first deformable portion to a second location within the first deformable portion of the flexible damping member.

Example 398. The system of example 395, wherein, after stress is applied at the first location, at least the portion of the abating substance is further configured to move longitudinally and/or radially from the first location to a third location within a second deformable portion of the flexible damping member.

Example 399. The system of any one of examples 359 to 398, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

Example 400. The system of any one of examples 359 to 399, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

Example 401. The system of example 400, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

### R. Examples 402-473: Methods for Treating a Patient Having a Condition

Example 402. A method for treating a patient having a condition comprising:
(a) determining or having determined whether the patient has an elevated pulse pressure, elevated pulse wave intensity/FCWI, and a history of a microbleed, blood brain barrier dysregulation and/or increased permeability, systemic inflammation, blood brain barrier inflammation, neuroinflammation, increased level of at least one circulating or cerebral spinal fluid (CSF) cytokine, increased level of reactive oxygen species, or a combination thereof by:
   (i) obtaining or having obtained information which indicates that the patient has the elevated pulse pressure, elevated pulse wave intensity/FCWI, and has previously had symptoms of a microbleed, blood brain barrier dysregulation and/or increased permeability, systemic inflammation, blood brain barrier inflammation, neuroinflammation, increased level of at least one circulating or CSF cytokine, reactive oxygen species, or a combination thereof and/or was previously diagnosed with a microbleed, blood brain barrier dysregulation and/or increased permeability, systemic inflammation, blood brain barrier inflammation, neuroinflammation, increased level of at least one circulating or CSF cytokine, increased reactive oxygen species, or a combination thereof; and/or
   (ii) monitoring or having monitored the subject for the elevated pulse pressure, elevated pulse wave intensity/FCWI, and symptoms of a microbleed, blood brain barrier dysregulation and/or increased permeability, systemic inflammation, blood brain barrier inflammation, neuroinflammation, increased level of at least one circulating cytokine, increased reactive oxygen species, or a combination thereof; and
(b) if the patient has previously had symptoms of a microbleed, blood brain barrier dysregulation and/or increased permeability, systemic inflammation, blood brain barrier inflammation, neuroinflammation, increased level of at least one circulating cytokine, increased reactive oxygen species, or a combination thereof, was previously diagnosed with a microbleed, blood brain barrier dysregulation and/or increased permeability, systemic inflammation, blood brain barrier inflammation, neuroinflammation, increased level of at least one circulating cytokine, increased reactive oxygen species, or a combination thereof, and/or symptoms of a microbleed, systemic inflammation, blood brain barrier inflammation, neuroinflammation, increased level of at least one circulating cytokine, or a combination thereof were monitored, then
   (i) providing a cell-based therapy, a gene-based therapy, a microbiome-based therapy, or a peptide-based therapy to the patient, and
   (ii) providing a device for treating and/or preventing one or more effects of the condition,
the device comprising —
   a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
   an abating substance disposed within the one or more at least partially deformable portions of the sidewall configured to move longitudinally and/or radially within one partially deformable portion in response to pulsatile blood flow within the blood vessel;
(c) if the patient has not had symptoms of a microbleed, blood brain barrier dysregulation and/or increased permeability, systemic inflammation, blood brain barrier inflammation, neuroinflammation, increased level of at least one circulating cytokine, increased reactive oxygen species, or a combination thereof, was not previously diagnosed with a microbleed, blood brain barrier dysregulation and/or increased permeability, systemic inflammation, blood brain barrier inflammation, neuroinflammation, increased level of at least one circulating cytokine, increased reactive oxygen species or a combination thereof, then providing the device for treating and/or preventing one or more effects of the condition.

Example 403. The method of example 402, wherein the elevated pulse pressure is a pulse pressure of at least 50 mmHg.

Example 404. The method of example 402 or example 403, wherein the increased level of at least one circulating cytokine in (b) is a level at least about 5% greater than the level in (c).

Example 405. The method of any one of examples 402 to 404, wherein the at least one cytokine is selected from the group consisting of VCAM-1, ICAM-1, TNFa, TGF-β, IL-6, IL-8, IL-1β, IL-12, and NF-_{K}B.

Example 406. The method of example 402, wherein the cell-based therapy is stem cell therapy or progenitor cell therapy.

Example 407. The method of example 406, wherein the stem cell therapy is allogenic or autologous human mesenchymal stem cell therapy.

Example 408. The method of example 406, wherein the stem cell therapy is autologous bone-marrow derived or adipose-derived human mesenchymal stem cell therapy.

Example 409. The method of example 406, wherein the stem cell therapy is autologous umbilical cord blood-derived or placenta-derived human mesenchymal stem cell therapy.

Example 410. The method of example 402, wherein the gene-based therapy includes at least one viral vector.

Example 411. The method of example 410, wherein at least one viral vector is an adeno-associated viral (AAV) vector.

Example 412. The method of example 411, wherein the AAV vector includes a nucleic acid that reduces or prevents expression of APOE4.

Example 413. The method of example 411, wherein the AAV vector includes a nucleic acid that encodes NGF or hTERT.

Example 414. The method of example 402, wherein the microbiome-based therapy includes providing one or more fecal microbiota transplant therapeutic agents to the subj ect.

Example 415. The method of example 414, wherein the fecal microbiota transplant therapeutic agents are capsules containing at least one microbiota organism derived or isolated from feces.

Example 416. The method of example 402, wherein the microbiome-based therapy includes providing a probiotic to the subject.

Example 417. The method of example 416, wherein the probiotic is Omni-Biotic stress repair.

Example 418. The method of example 402, wherein the microbiome-based therapy includes providing a sodium oligo-mannurarate remodelling agent to the subject.

Example 419. The method of example 418 wherein the sodium oligo-mannurarate remodelling agent is GV-971.

Example 420. The method of example 402, wherein the peptide-based therapy includes an ETB receptor agonist (e.g., IRL-1620) or a peptide derivative or variant of APC.

Example 421. The method of example 420, wherein the peptide derivative or variant of APC includes an amino acid sequence which differs from an amino acid sequence of wild-type APC.

Example 422. The method of example 420 or example 421, wherein the peptide derivative or variant of APC is 3K3A-APC.

Example 423. The method of any one of examples 402 to 422, wherein the therapy prevents and/or reduces expression of APOE4 or increases the expression of APOE2 or APOE3, promotes expression of one or more neurotrophic factors, prevents and/or reduces an abnormal biome from developing in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents or reduces dysfunction/damage and/or death of a neuron, prevents or reduces blood brain barrier dysregulation and/or increase permeability, and/or reduces and/or prevents insulin resistance.

Example 424. The method of any one of examples 402 to 423, wherein the therapy is provided at a first dosage which is lower than a second dosage that is provided in the absence of the device.

Example 425. The method of any one of examples 402 to 424, wherein the therapy is provided at a first dosing regimen which is less than a second dosing regimen that is provided in the absence of the device.

Example 426. The method of any one of examples 402 to 425, wherein the therapy is provided via a first route which is different than a second route that is provided in the absence of the device.

Example 427. The method of any one of examples 402 to 426, wherein the therapy is provided by administering the therapy to the subject in need thereof.

Example 428. The method of any one of examples 402 to 427, wherein step (b) is performed after step (a) and before step (c).

Example 429. The method of any one of examples 402 to 428, wherein step (c) is performed after step (a) and before step (b).

Example 430. The method of any one of examples 402 to 429, wherein the condition is neurodegeneration.

Example 431. The method of example 430, wherein neurodegeneration further comprises Alzheimer’s disease, dementia, and/or cognitive impairment.

Example 432. The method of any one of examples 402 to 431, wherein the inner surface and/or an outer surface has a generally cylindrical shape or an undulating shape that undulates in a longitudinal direction.

Example 433. The method of any one of examples 402 to 432, wherein the device has a low-profile state and a deployed state, and when in the deployed state, the sidewall is generally tubular.

Example 434. The method of any one of examples 402 to 433, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 435. A method for treating a patient having a condition comprising:
(a) determining or having determined whether the patient has an elevated pulse pressure or elevated pulse wave intensity/FCWI and a history of a reduced level of one or more circulating neurotrophic factors:
   (i) obtaining or having obtained information which indicates that the patient has the elevated pulse pressure and the reduced level of one or more circulating neurotrophic factors and/or was previously diagnosed with the reduced level of one or more circulating neurotrophic factors; and/or
   (ii) monitoring or having monitored the subject for the elevated pulse pressure or elevated pulse wave intensity/FCWI and the reduced level of one or more circulating neurotrophic factors; and
(b) if the patient has previously had the reduced level of one or more circulating neurotrophic factors, was previously diagnosed with the reduced level of one or more circulating neurotrophic factors, and/or the reduced level of one or more circulating neurotrophic factors was monitored, then
   (i) providing a cell-based therapy to the patient, and
   (ii) providing a device for treating and/or preventing one or more effects of the condition,
the device comprising —
   a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
   an abating substance disposed within the one or more at least partially deformable portions of the sidewall configured to move longitudinally and/or radially within one partially deformable portion in response to pulsatile blood flow within the blood vessel;
(c) if the patient has not had the reduced level of one or more circulating neurotrophic factors, was not previously diagnosed with the reduced level of one or more circulating neurotrophic factors, then providing the device for treating and/or preventing one or more effects of the condition.

Example 436. The method of example 435, wherein the elevated pulse pressure is a pulse pressure of at least 50 mmHg.

Example 437. The method of example 435 or example 436, wherein the reduced level of one or more circulating neurotrophic factors in (b) is a level at least about 5% greater than the level in (c).

Example 438. The method of any one of examples 435 to 437, wherein the one or more circulating neurotrophic factors is selected from the group consisting of NGF and BDNF.

Example 439. The method of example 435, wherein the cell-based therapy is stem cell therapy or progenitor cell therapy.

Example 440. The method of example 439, wherein the stem cell therapy is allogenic or autologous human mesenchymal stem cell therapy.

Example 441. The method of example 439, wherein the stem cell therapy is autologous bone-marrow derived or adipose-derived human mesenchymal stem cell therapy.

Example 442. The method of example 439, wherein the stem cell therapy is autologous umbilical cord blood-derived or placenta-derived human mesenchymal stem cell therapy.

Example 443. The method of any one of examples 435 to 442, wherein the cell-based therapy promotes expression of one or more neurotrophic factors.

Example 444. The method of any one of examples 435 to 443, wherein the cell-based therapy is provided at a first dosage which is lower than a second dosage that is provided in the absence of the device.

Example 445. The method of any one of examples 435 to 444, wherein the cell-based therapy is provided at a first dosing regimen which is less than a second dosing regimen that is provided in the absence of the device.

Example 446. The method of any one of examples 435 to 445, wherein the cell-based therapy is provided via a first route which is different than a second route that is provided in the absence of the device.

Example 447. The method of any one of examples 435 to 446, wherein the cell-based therapy is provided by administering the cell-based therapy to the subject in need thereof.

Example 448. The method of any one of examples 435 to 447, wherein step (b) is performed after step (a) and before step (c).

Example 449. The method of any one of examples 435 to 448, wherein step (c) is performed after step (a) and before step (b).

Example 450. The method of any one of examples 435 to 449, wherein the condition is neurodegeneration.

Example 451. The method of example 450, wherein neurodegeneration further comprises Alzheimer’s disease, dementia, and/or cognitive impairment.

Example 452. The method of any one of examples 435 to 451, wherein the inner surface and/or an outer surface has a generally cylindrical shape or an undulating shape that undulates in a longitudinal direction.

Example 453. The method of any one of examples 435 to 452, wherein the device has a low-profile state and a deployed state, and when in the deployed state, the sidewall is generally tubular.

Example 454. The method of any one of examples 435 to 453, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

Example 455. A method for treating a patient having a condition comprising:
(a) determining or having determined whether the patient has an elevated pulse pressure or elevated pulse wave intensity/FCWI, and an expression of APOE4 by:
   (i) obtaining or having obtained information which indicates that the patient has the elevated pulse pressure or elevated pulse wave intensity/FCWI and has previously had the expression of APOE4 and/or was previously diagnosed with the expression of APOE4; and/or
   (ii) monitoring or having monitored the subject for the elevated pulse pressure or elevated pulse wave intensity/FCWI and the expression of APOE4; and
(b) if the patient has previously had the expression of APOE4, was previously diagnosed with the expression of APOE4, and/or the expression of APOE4 was monitored, then
   (i) providing a gene-based therapy to the patient, and
   (ii) providing a device for treating and/or preventing one or more effects of the condition,
the device comprising —
   a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
   an abating substance disposed within the one or more at least partially deformable portions of the sidewall configured to move longitudinally and/or radially within one partially deformable portion in response to pulsatile blood flow within the blood vessel;
(c) if the patient has not had symptoms of the expression of APOE4, was not previously diagnosed with the expression of APOE4, then providing the device for treating and/or preventing one or more effects of the condition.

Example 456. The method of example 455, wherein the elevated pulse pressure is a pulse pressure of at least 50 mmHg.

Example 457. The method of example 455 or example 456, wherein the expression of APOE4 in (b) is a level at least about 5% greater than the level in (c).

Example 458. The method of example 455, wherein the gene-based therapy includes at least one viral vector.

Example 459. The method of example 458, wherein at least one viral vector is an adeno-associated viral (AAV) vector.

Example 460. The method of example 459, wherein the AAV vector includes a nucleic acid that reduces or prevents expression of APOE4.

Example 461. The method of example 455, wherein the peptide derivative of APC includes an amino acid sequence which differs from an amino acid sequence of wild-type APC.

Example 462. The method of any one of examples 455 to 461 wherein the gene-based therapy prevents and/or reduces expression of APOE4 or increases the expression of APOE2 or APOE3.

Example 463. The method of any one of examples 455 to 462, wherein the gene-based therapy is provided at a first dosage which is lower than a second dosage that is provided in the absence of the device.

Example 464. The method of any one of examples 455 to 463, wherein the gene-based therapy is provided at a first dosing regimen which is less than a second dosing regimen that is provided in the absence of the device.

Example 465. The method of any one of examples 455 to 464, wherein the gene-based therapy is provided via a first route which is different than a second route that is provided in the absence of the device.

Example 466. The method of any one of examples 455 to 465, wherein the gene-based therapy is provided by administering the therapy to the subject in need thereof.

Example 467. The method of any one of examples 455 to 466, wherein step (b) is performed after step (a) and before step (c).

Example 468. The method of any one of examples 455 to 467, wherein step (c) is performed after step (a) and before step (b).

Example 469. The method of any one of examples 455 to 468, wherein the condition is neurodegeneration.

Example 470. The method of example 469, wherein neurodegeneration further comprises Alzheimer’s disease, dementia, and/or cognitive impairment.

Example 471. The method of any one of examples 455 to 470, wherein the inner surface and/or an outer surface has a generally cylindrical shape or an undulating shape that undulates in a longitudinal direction.

Example 472. The method of any one of examples 455 to 471, wherein the device has a low-profile state and a deployed state, and when in the deployed state, the sidewall is generally tubular.

Example 473. The method of any one of examples 455 to 472, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

In addition, the following prophetic examples are illustrative of several embodiments of the present technology.

### S. Example 474: Clinical Study Design (Prophetic Example)

Implantable devices will be positioned at, near, around, within, or in place of at least a portion of a subject’s artery in accordance with the present technology. After the implantable devices have been positioned, subjects who received the implantable device will be randomized into one of at least two groups: Group A - placebo, and Group B - biologic therapeutic agent. The placebo will be an experimentally appropriate placebo useful for distinguishing any specific effects of the drug, such as the pharmaceutically acceptable carrier for the active component in the biologic therapeutic agent. The dose of the placebo will be comparable to the amount of pharmaceutically acceptable carrier that subjects in Group B receive. Group B can include two or more subgroups, with subjects being randomly assigned to each subgroup. While the subjects in each of these Group B subgroups each ultimately receive the same biologic therapeutic agent, the dose, route of administration, dosing regimen, or other parameters associated with a therapeutic protocol can be altered.

### T. Example 475: Clinical Study Design (Prophetic Example)

A biologic therapeutic agent will be delivered to a subject at a pre-specified dose, route of administration, frequency, and duration. After the biologic therapeutic agent has been delivered to the subject, subjects will be randomized into one of at least two groups: Group A -sham, and Group B - implantable device. For those subjects in Group B, implantable devices will be positioned at, near, around, within, or in place of at least a portion of a subject’s artery in accordance with the present technology. The sham treatment for Group A includes the delivery methods associated with delivery of the implantable device used for Group B, although the implantable device will not be delivered to the subjects in Group A.

In an alternative study, subjects will be randomized into one of at least two groups: Group A - sham, and Group B - implantable device. For those subjects in Group B, implantable devices will be positioned at, near, around, within, or in place of at least a portion of a subject’s artery in accordance with the present technology. The sham treatment for Group A includes the delivery methods associated with delivery of the implantable device used for Group B, although the implantable device will not be delivered to the subjects in Group A. After Groups A and B have been selected and implantable devices (Group B) or sham treatments (Group A) have been provided, a biologic therapeutic agent will be delivered to a subject at a pre-specified dose, route of administration, frequency, and duration.

In another alternative study, subjects will be randomized into one of at least two groups: Group A - sham, and Group B - implantable device. For those subjects in Group B, implantable devices will be positioned at, near, around, within, or in place of at least a portion of a subject’s artery in accordance with the present technology, and a biologic therapeutic agent will be delivered to the subjects at a pre-specified dose, route of administration, frequency, and duration at the time of implantation. The sham treatment for Group A includes the delivery methods associated with delivery of the implantable device used for Group B, although the implantable device will not be delivered to the subjects in Group A. Group A will also be delivered a biologic therapeutic agent at a pre-specified dose, route of administration, frequency, and duration at the time of sham treatment.

### U. Example 476: Stem And/Or Progenitor Cell Study (Prophetic Example)

Co-Culture Response to Reduced Pulsatility: Immortalized human cerebral microvascular endothelial cells, such as HCMEC-SV40 cells, will be cultured. Additionally, stem or progenitor cells, in this example human bone marrow derived mesenchymal stem cells, will be obtained and cultured. The co-culture compatibility of cerebral microvascular endothelial cells and human bone marrow derived mesenchymal stem cells will be confirmed and a medium that suits both cell types will be identified.

Cerebral microvascular endothelial cells will be cultured in the determined co-culture medium and cyclically stretched -15% (to simulate elevated pulse pressure), as described in Gangoda et al., 2018. Separately, the stem cells will be cultured in the co-culture medium without stretch and pre-stained with PKH26 (excitation 551 nm, emission 567 nm), a fluorescent membrane-labelling dye with half-life elution from cells of at least 100 days, so they can be visually differentiated from the cerebral microvascular endothelial cells.

After the cerebral endothelial cells have been cyclically stretched, half of the cerebral endothelial cell cultures will be reduced to ~5% stretch (to simulate normal pulse pressure) while the other half of the cerebral endothelial cell cultures will remain at ~ 15% stretch (elevated pulse pressure). This will represent +/- application of the device of the present disclosure, respectively. Next, the PKH26-stained stem cells will be added to the 5% stretch cerebral endothelial cell cultures and to the 15% stretch cerebral microvascular endothelial cell cultures.

The time point for co-culture culture analysis may then be determined based on comparative co-culture morphological appearances, comparative co-culture secreted protein levels, or other related method to assess characteristics of the high and normal pulse pressure cocultures.

Once the final co-culture time point is determined, each co-culture will be analysed as follows:

(I) Conditioned medium will be collected and ELISAs will be used to measure the secreted expression of inflammatory and amyloid pathway proteins.

(II) Cells will be counted (final time point secreted protein levels will be normalized to this) then sorted based on PKH26 signal to determine abundance and potential proliferation of stem cells and cerebral microvascular endothelial cells.

(III) Once sorted, stem cells and cerebral microvascular endothelial cells may be separately assessed for viability using a lactate dehydrogenase release (LDH) assay.

(IV) Western blots of cerebral microvascular endothelial cells can be run to quantify cellular content of inflammatory and amyloid pathway proteins.

(V) Western blots of stem cells can be run to quantify cellular content of inflammatory and differentiation proteins.

### V. Example 477: Stem And/Or Progenitor Cell Study (Prophetic Example)

Wound Healing Response to Reduced Pulsatility: Cultures of cerebral microvascular endothelial cells, such as HCMEC-SV40 cells, in the co-culture medium will be grown to confluence and cyclically stretched ~15%, as described in Gangoda et al., 2018. After the cerebral endothelial cells have been cyclically stretched, a scrape/wound will be made in each cerebral microvascular endothelial cell culture. Next, half of the cerebral endothelial cell cultures will be reduced to ~5% stretch, while the other half will remain at -15% stretch. PKH26-stained stem/progenitor cells will be added to the 5% stretch cerebral endothelial cell cultures and to the 15% stretch cerebral microvascular endothelial cell cultures. Fluorescence microscopy (time course) will be used to visualize wound healing. PKH26 fluorescence signal will determine if stem/progenitor cells migrate to close the wound, if the wound is instead closed by cerebral microvascular endothelial cell proliferation, or if the wound remains unhealed under each stretch condition. Cells may then be sorted based on PKH26 signal, and western blots of stem/progenitor cells will be run to quantify cellular content of inflammatory and differentiation markers.

### W. Example 478: Gene Therapy Study (Prophetic Example)

Influence of Reduced Pulsatility on endothelial gene therapy: Cerebral microvascular endothelial cells will be cultured and cyclically stretched ~15%, as described in Gangoda et al., 2018. After the cerebral endothelial cells have been cyclically stretched, confluence will be determined, and samples of conditioned media will be collected. Half of the cerebral endothelial cell cultures will be reduced to ~5% stretch, while the other half of the cerebral endothelial cell cultures will remain at -15% stretch. This will represent +/- application of the device of the present disclosure, respectively. Then, a viral vector containing a reporter gene (e.g., fluorescent protein) will be added to both the 5% stretch and 15% stretch endothelial cell cultures. Alternatively, a viral vector containing a gene for a neurotrophic factor may be used, and transgene secretion levels could be measured. After incubation with virus, cells will be washed and continue to be cultured under 5% or 15% stretch. Cells will then be assessed for transduction efficiency, as well as fluorescence intensity.

Alternatively, for a secretory transgene, conditioned media from the 5% and 15% stretch endothelial cell cultures will be collected, normalized for cell density, and quantified via western blot, ELISA, or Luminex bead-based assay to determine relative transgene protein product concentration.

### X. Example 479: Gene Therapy Study (Prophetic Example)

Influence of ReducedPulsatility on Neuronal Gene Therapy: Immortalized human cerebral microvascular endothelial cells, such as HCMEC-SV40 cells, will be tested for their compatibility with modified Dulbecco’s modified Eagle’s medium. If successful, cerebral microvascular endothelial cells will be equally seeded in this medium to make 12 cultures. Half of these cultures will be cyclically stretched 5% and the other half will be cyclically stretched 15%, as described in Gangoda et al, 2018. Separately, human cortical neurons will be cultured in the same medium without stretch. Additionally, AAV-GFP serotype 9 (adeno-associated virus type 9 with a gene fluorescent protein transgene) viral particles will be generated and purified.

After the cerebral endothelial cells have been stretched, the conditioned media from the 5% stretch cultures will be collected and pooled, and the media from the 15% stretch cultures will be collected and pooled.

Media from human cortical neuron cultures (no stretch) will then be aspirated and replaced by the pooled conditioned media from the cerebral microvascular endothelial cells:
(I) Human cortical neuron cultures (no stretch) + 5% stretch conditioned medium
(II) Human cortical neuron cultures (no stretch) + 15% stretch conditioned medium

After a period of incubation in conditioned media, AAV-GFP viral particles will added to the human cortical neuron cultures (no stretch) at a titer of ~5 x 10¹⁰ GC/ml and incubated:
(I) Human cortical neuron cultures + 5% stretch conditioned medium + AAV-GFP
(II) Human cortical neuron cultures + 5% stretch conditioned medium (AAV negative control)
(III) Human cortical neuron cultures + 15% stretch conditioned medium + AAV-GFP
(IV) Human cortical neuron cultures + 15% stretch conditioned medium (AAV negative control)

Conditioned media will then be aspirated, human cortical neuron cells will be washed with PBS, and 5% or 15% stretch conditioned medium (without virus) will be given to the respective human cortical neuron cultures (no stretch). After 1 week, fluorescence microscopy will be used to quantify GFP transfection efficiency and GFP signal intensity. Cell viability can be quantified using an LDH assay. Western blots will be used to quantify GFP protein expression.

### Y. Example 480: Microbiome Systemic Inflammation Reduction Study (Prophetic Example)

Influence of Reduced Pulsatility on microbiome therapy efficacy: Cerebral microvascular endothelial cells will be cultured and cyclically stretched ~15%, as described in Gangoda et al., 2018. Cultures will either be grown in standard media (no systemic inflammation) or will be grown with media supplemented with IL-1β and TNFa (simulating systemic inflammation). After the cerebral endothelial cells have been cyclically stretched (with and without supplemental IL-1β and TNFa), confluence will be determined. Half of the cerebral endothelial cell cultures will be reduced to ~5% stretch while the other half of the cerebral endothelial cell cultures will remain at -15% stretch. This will represent +/- application of the device of the present disclosure, respectively. An overview of this scheme is summarized in Table 1 below.

Endothelial cells will be assessed for viability and proliferation to determine if the combination therapy culture (italics) is healthiest over time.

TABLE 1

Device and Microbiome Therapeutic Agent Combinations Modelled in Example 480

| | | | | |
|---|---|---|---|---|
| Pressure/stretch | normal/~5% | normal/~5% | elevated/~15% | elevated/~15% |
| media | standard | +IL-6 and TNFα | standard | +IL-6 and TNFα |
| represents | device + reduced systemic inflammation | device alone | reduced systemic inflammation alone | untreated (no therapy) |

### Z. Example 481: Peptide-Based Therapy Study (Prophetic Example)

Influence of Reduced Pulsatility on Peptide Therapy (e.g., APC or IRL-1620): Immortalized human cerebral microvascular endothelial cells, such as HCMEC-SV40 cells, will be cultured and cyclically stretched ~15%, as described in Gangoda et al, 2018. After the cerebral endothelial cells have been cyclically stretched, confluence will be noted, and samples of conditioned media will be collected. Half of the cerebral endothelial cell cultures will be reduced to ~5% stretch, while the other half will remain at 15% stretch. This will represent +/application of the device described herein, respectively.

Next, combination therapy will be administered as follows:
(I) Cerebral endothelial cells reduced to 5% stretch + peptide drug
(II) Cerebral endothelial cells maintained at 15% stretch + peptide drug.

At a predetermined time after drug dosing, the following assays will be performed for each condition:

(I) Conditioned medium will be collected, and ELISAs will be used to measure the expression of inflammatory and amyloid pathway proteins (normalized to confluence).

(II) Cell viability may be quantified using an LDH assay.

(III) Western blots of cerebral microvascular endothelial cells will be used to quantify cellular content of inflammatory and amyloid pathway proteins.

### AA. Example 482: In Vivo Surgically Induced Animal Model Study Design (Prophetic Example)

In Vivo Model of Device. Male WT mice (e.g., C57B⅙ and BALB/c) and/or Alzheimer disease model mice will be acclimatized for 2 weeks prior to the experiment. Animals will be kept under a standard condition with room temperature at 21-23° C., 30-70% relative humidity, and a 12:12 h light:dark cycle. Chow and water are available ad libitum.

After 2 weeks of acclimatization, mice will be weighed (weekly throughout the full study). Right carotid and left carotid blood flow and pulse pressure will be measured using any standard method in the art, e.g., a non-invasive Doppler Flow Velocity System (Scintica Instrumentation, Inc.), or invasive pressure catheter (Millar). Mice will then split into groups with comparable average weights. Mice will undergo transverse aortic constriction (TAC) surgery, which involves placing a constriction around the transverse aorta, limiting left ventricular (LV) outflow—thus creating a pressure overload in the LV and inducing an elevated pulse pressure. The TAC surgery will be performed using either (i) a nylon suture to induce a sustained (or permanent) elevated pulse pressure, or (ii) an absorbable suture (e.g., Polyglactin 910, which lasts 14-21 days at maximum strength) to induce a transient (or temporary) elevated pulse pressure, which will gradually dissipate as the suture dissolves, representing the reduction of elevated pulse pressure and associated cerebral microbleeds that occurs over time over time. The specific absorption rate of the selected absorbable suture and its effect on pulse pressure over time in mice will be confirmed. The TAC mice receiving the absorbable suture models the damping device.

Next, the following groups of mice will be examined to confirm that less Alzheimer-related pathology/more blood brain barrier (BBB) integrity is seen in the absorbable suture mice as compared to the nylon suture TAC mice, and to support the damping device’s mechanism of action (reducing elevated pulse pressure).
(I) WT mice, TAC with nylon suture
(II) WT mice, TAC with absorbable suture
(III) Alzheimer mice, TAC with nylon suture
(IV) Alzheimer mice, TAC with absorbable suture

The time point for group comparisons after the TAC procedure may be determined based on when optimal, contrasting pulse pressures are achieved with nylon versus absorbable sutures. In certain aspects, the experiment start point may be approximately 6 weeks post-TAC.

At the time point for group comparisons, mice will be weighed, and blood flow and pulse pressure will be recorded. Mice will then be injected with Evans blue dye (excitation 470/540 nm, emission 680 nm) or any other fluorescent tracer known in the art, undergo perfusion, then sacrificed. Whole brains will be extracted, and brain tissue will immediately be fixed (or snap frozen depending on what the tissue will be used for) and sectioned. Mouse hearts may be collected and weighed. Fixed ipsilateral and contralateral histological brain sections will be stained with Prussian Blue to quantify microbleeds as described in Supplement 1 of Montgolfier et al. (see above) or confocal imaged for fluorescence distribution. Immunohistochemistry to visualize expression of BBB markers such as tight junction proteins may also be considered as an indicator of BBB integrity. MMP (a marker for increased BBB permeability) and NF-kB (an inflammatory marker associated with Alzheimer’s disease) protein expression may also be measured in brain tissue. Amyloid-beta deposition in neural tissue may be quantified in Alzheimer’s model mice as well. Finally, immunohistochemistry of fibrin in brain sections may be conducted to visualize clotting of blood components that have leaked through the BBB into the neuropil.

In Vivo Model of Combination Therapy. Following successful completion of the proof-of-concept experiments described above, the benefit of reducing elevated pulse pressure on the efficacy of drug or cell therapies will be studied in vivo. First, as detailed previously, WT or Alzheimer’s model mice will undergo TAC surgery with either a nylon suture or absorbable suture.

At the time point for group comparison after the TAC procedure discussed above, the mice will be dosed (via intracarotid or tail vein) with fluorescently labeled stem/progenitor cells or a drug described herein (e.g., a “biologic therapeutic agent”, “biologic agent”, “biologic therapy”, cell-based therapeutic agent, gene-based therapeutic agent or a gene product produced therefrom, a peptide-based therapeutic agent, and/or a microbiome-based therapy); or a therapeutic agent such as those described or disclosed in International Application Publication WO2020117560, which is hereby incorporated by reference as if fully support therein.

For Example, the mice may be dosed with a therapeutic agent (cell therapy or drug) as shown below:
(I) TAC with nylon suture + therapeutic agent
(II) TAC with absorbable suture + therapeutic agent

Exemplary therapies that may be tested according to this example include, but are not limited to, stem cells, progenitor cells, IRL-1620, etodolac, elenbecestat, lanabecestat, solanezumab, CNP520, and crenezumab.

At a time point for group comparisons, mice will be weighed, and blood flow and pulse pressure will be recorded. Mice will then be injected with Evans blue dye/fluorescent tracer, undergo perfusion, then sacrificed. Whole brains will be extracted, and brain tissue will immediately be fixed (or snap frozen depending on what the tissue will be used for) and sectioned. Mouse hearts will be collected and weighed. Fixed ipsilateral and contralateral histological brain sections will be stained with Prussian Blue to quantify microbleeds, see Supplement 1 of Montgolfier et al. (see above), or confocal imaged for fluorescence distribution. The PKH26/CFDA-SE (fluorescence) signal may reveal abundance of recruited fluorescence-labeled stem or progenitor cells in cerebral microvasculature. Evans blue/fluorescent tracer signal may show BBB leakage. Immunohistochemistry to visualize expression of BBB markers such as tight junction proteins could also be considered as an indicator of BBB integrity. MMP (a marker for increased BBB permeability) and NF-kB (an inflammatory marker associated with Alzheimer’s disease and stem cell dysfunction) protein expression could also be measured in brain tissue. Finally, immunohistochemistry of fibrin in brain sections could be conducted to visualize clotting of blood components that have leaked through the BBB into the neuropil.

The in vivo model described herein may be used to test any therapy administered in combination with reduction of elevated pulse pressure (effect of the damping device), including biologic and non-biologic therapies. These combination therapy experiments will confirm whether the absorbable suture TAC mice + therapeutics show less disrupted BBB than the nylon suture TAC mice + therapeutics. If so, this will support the potential for the device to synergistically enhance the efficacy of such therapeutics.

### BB. Example 482: In Vivo Chemically Induced Pulse Pressure Study Design (Prophetic Example)

As an alternative to the TAC surgically induced model of elevated pulse pressure discussed above, a chemically induced animal model of elevated pulse pressure may also be used to test biological therapeutics described herein (e.g., a “biologic therapeutic agent”, “biologic agent”, “biologic therapy”, cell-based therapeutic agent, gene-based therapeutic agent or a gene product produced therefrom, a peptide-based therapeutic agent, and/or a microbiome-based therapy) or other therapeutic agents such as those described or disclosed in International Application Publication WO2020117560, which is hereby incorporated by reference as if fully support therein. Such models can be generated by treating the animals (e.g., rats) with warfarin/vitamin K1 (WVK) in accordance with Essalihi et al., A new model of isolated systolic hypertension induced by chronic warfarin and vitamin K1 treatment, Am J Hypertens. 2003 Feb;16(2): 103-10 (doi: 10.1016/s0895-7061(02)03204-1). The WVKtreatment induces isolated systolic hypertension and thus, elevated pulse pressure. This elevated pulse pressure can be reduced with subsequent treatment with darusentan (DAR) and/or acetazolamide (ACTZ) treatment in accordance with Essalihi et al., Regression of medial elastocalcinosis in rat aorta: a new vascular function for carbonic anhydrase, Circulation. 2005 Sep 13;112(11): 1628-35 (doi: 10.1161/CIRCULATIONAHA.104.528984).

To compare cerebral vascular health animals experiencing elevated pulse pressure compared to when that pulse pressure is reduced, animals will be treated in one or more of the groups that follow:

| | | |
|---|---|---|
| Treatment Group | Treatment | Duration |
| Maintained elevated pulse pressure arm | WVK treatment | 8 weeks (or other suitable predetermined duration) |
| Reduced elevated pulse pressure arm | WVK treatment DAR and/or ACTZ treatment | 4 weeks (or other suitable predetermined duration) 4 weeks (or other suitable predetermined duration) |
| Maintained elevated pulse pressure + Cell therapy arm | WVK treatment cell therapy treatment | 8 weeks (or other suitable predetermined duration) 4 weeks (or other suitable predetermined duration) |
| Maintained elevated pulse pressure +Drug arm | WVK treatment drug treatment | 8 weeks (or other suitable predetermined duration) 4 weeks (or other suitable predetermined duration) |
| Reduced elevated pulse pressure + Cell therapy arm | WVK treatment DAR and/or ACTZ treatment + cell therapy treatment | 4 weeks (or other suitable predetermined duration) 4 weeks (or other suitable predetermined duration) |
| Reduced elevated pulse pressure + Drug arm | WVK treatment DAR and/or ACTZ treatment + drug treatment | 4 weeks (or other suitable predetermined duration) 4 weeks (or other suitable predetermined duration) |

After the treatment period, cognition, microbleeds, BBB permeability, neuroinflammation, neurodegeneration, and expression of BBB tight junctions could be quantified and compared for each group of rats detailed in the table above

The study design described herein may be used to test any therapy intended to be administered in combination with reduction of elevated pulse pressure (effect of the damping device), including biologic and non-biologic therapies described herein and in International Application Publication WO2020117560, which is hereby incorporated by reference as if fully support therein. Exemplary cell therapy or drug treatments that may be tested using this study design include, but are not limited to, stem cells, progenitor cells, IRL-1620, etodolac, elenbecestat, lanabecestat, solanezumab, CNP520, and crenezumab.

### VIII. Conclusion

Although many of the embodiments are described above with respect to systems, devices, and methods for treating and/or slowing the progression of vascular and/or age-related neurological conditions (e.g., dementia) via combinatorial therapeutic agents (e.g., drugs) and intravascular methods, the technology is applicable to other applications and/or other approaches, such as surgical implantation of one or more damping devices and/or treatment of blood vessels other than arterial blood vessels supplying blood to the brain, such as the abdominal aorta, in combination with one or more drugs. Any appropriate site within a blood vessel may be treated including, for example, the ascending aorta, the aortic arch, the brachiocephalic artery, the right subclavian artery, the left subclavian artery, the left common carotid artery, the right common carotid artery, the internal and external carotid arteries, and/or branches of any of the foregoing. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIG. 2A-20.

The above detailed descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for treating and/or preventing one or more effects of a condition in a subject in need thereof, the method comprising:
providing a device for treating and/or preventing one or more effects of the condition, and configured to be placed in apposition with a blood vessel, the device comprising —
a flexible damping member forming a generally tubular structure having an inner surface and an outer surface, the inner surface formed of a sidewall having one or more at least partially deformable portions, and
an abating substance disposed within the one or more at least partially deformable portions of the sidewall configured to move longitudinally and/or radially within one partially deformable portion in response to pulsatile blood flow within the blood vessel; and
providing a biologic therapy that treats or slows one or more effects of the condition in combination with the device.

2. The method of claim 1, wherein the biologic therapy is a cell-based therapy, a gene-based therapy, a microbiome-based therapy, and/or a peptide-based therapy.

3. The method of claim 2, wherein the cell-based therapy is stem cell therapy or progenitor cell therapy.

4. The method of claim 3, wherein the stem cell therapy is allogenic or autologous human mesenchymal stem cell therapy.

5. The method of claim 3, wherein the stem cell therapy is autologous bone-marrow derived or adipose-derived human mesenchymal stem cell therapy.

6. The method of claim 3, wherein the stem cell therapy is autologous umbilical cord blood-derived or placenta-derived human mesenchymal stem cell therapy.

7. The method of claim 2, wherein the gene-based therapy includes at least one viral vector.

8. The method of claim 7, wherein the at least one viral vector is an adeno-associated viral (AAV) vector.

9. The method of claim 8, wherein the AAV vector includes a nucleic acid that reduces or prevents expression of APOE4.

10. The method of claim 8, wherein the AAV vector includes a nucleic acid that encodes NGF or hTERT.

11. The method of claim 2, wherein the microbiome-based therapy includes providing one or more fecal microbiota transplant therapeutic agents to the subject.

12. The method of claim 11, wherein the fecal microbiota transplant therapeutic agents are capsules containing at least one microbiota organism derived or isolated from feces.

13. The method of claim 2, wherein the microbiome-based therapy includes providing a probiotic to the subject.

14. The method of claim 13, wherein the probiotic is Omni-Biotic stress repair.

15. The method of claim 2, wherein the microbiome-based therapy includes providing a sodium oligo-mannurarate remodelling agent to the subject.

16. The method of claim 15, wherein the sodium oligo-mannurarate remodelling agent is GV-971.

17. The method of claim 2, wherein the peptide-based therapy includes IRL-1620 or a peptide derivative or a variant of activated protein kinase C (APC).

18. The method of claim 17, wherein the peptide derivative or variant of APC includes an amino acid sequence which differs from an amino acid sequence of wild-type APC.

19. The method of claim 17 or claim 18, wherein the peptide derivative or variant of APC is 3K3A-APC.

20. The method of any one of claims 2 to 19, wherein the biologic therapy prevents and/or reduces expression of APOE4 or increases the expression of APOE2 or APOE3, promotes expression of one or more neurotrophic factors, prevents and/or reduces an abnormal biome from developing in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents dysregulation/damage and/or death of a neuron, prevent dysregulation and/or damage to the blood brain barrier, and/or reduces and/or prevents insulin resistance.

21. The method of any one of claims 1 to 17, wherein the biologic therapy is provided at a first dosage which is lower than a second dosage that is provided in the absence of the device.

22. The method of any one of claims 1 to 21, wherein the biologic therapy is provided at a first dosing regimen which is less than a second dosing regimen that is provided in the absence of the device.

23. The method of any one of claims 1 to 22, wherein the biologic therapy is provided via a first route which is different than a second route that is provided in the absence of the device.

24. The method of any one of claims 1 to 23, wherein the biologic therapy is provided by administering the biologic therapy to the subject in need thereof.

25. The method of any one of claims 1 to 24, wherein the condition is neurodegeneration.

26. The method of claim 25, wherein neurodegeneration further comprises Alzheimer’s disease, dementia, and/or cognitive impairment.

27. The method of any one of claims 1 to 26, wherein the device has a low-profile state and a deployed state, and when in the deployed state, the sidewall is generally tubular.

28. The method of any one of claims 1 to 27, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

29. The method of claim 28, wherein when positioned in apposition with the blood vessel and a pulse wave travels through the blood vessel, the flexible damping member applies a stress at the first location along a length of the tubular structure.

30. The method of claim 29, wherein, after stress is applied at the first location, at least the portion of the abating substance moves longitudinally and/or radially along a length of the tubular structure.

31. The method of claim 29, wherein, after stress is applied at the first location, at least a portion of the abating substance is configured to move longitudinally and/or radially from a first location within a first deformable portion to a second location within the first deformable portion of the flexible damping member.

32. The method of claim 29, wherein, after stress is applied at the first location, at least the portion of the abating substance is further configured to move longitudinally and/or radially from the first location to a third location within a second deformable portion of the flexible damping member.

33. The method of any one of claims 1 to 32, wherein the inner surface and/or an outer surface has a generally cylindrical shape or an undulating shape that undulates in a longitudinal direction.

34. The method of any one of claims 1 to 33, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

35. The method of any one of claims 1 to 34, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

36. The method of claim 35, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

37. A method for treating and/or preventing one or more effects of a condition in a subject in need thereof, the method comprising:
providing a device for treating and/or preventing one or more effects of the condition and configured to be placed in apposition with a blood vessel, the device comprising
a flexible damping member forming a generally tubular structure having an inner surface and an outer surface, the inner surface formed of a sidewall having one or more at least partially deformable portions, and
an abating substance disposed within the one or more at least partially deformable portions of the sidewall configured to move longitudinally and/or radially within one partially deformable portion in response to pulsatile blood flow within the blood vessel; and
wherein a biologic therapy that treats or slows one or more effects of the condition has previously been provided to the subject in need thereof.

38. The method of claim 37, wherein the biologic therapy is a cell-based therapy, a gene-based therapy, a microbiome-based therapy, and/or a peptide-based therapy.

39. The method of claim 38, wherein the cell-based therapy is stem cell therapy or progenitor cell therapy.

40. The method of claim 39, wherein the stem cell therapy is allogenic or autologous human mesenchymal stem cell therapy.

41. The method of claim 39, wherein the stem cell therapy is autologous bone-marrow derived or adipose-derived human mesenchymal stem cell therapy.

42. The method of claim 39, wherein the stem cell therapy is autologous umbilical cord blood-derived or placenta-derived human mesenchymal stem cell therapy.

43. The method of claim 38, wherein the gene-based therapy includes at least one viral vector.

44. The method of claim 43, wherein the at least one viral vector is an adeno-associated viral (AAV) vector.

45. The method of claim 44, wherein the AAV vector includes a nucleic acid that reduces or prevents expression of APOE4.

46. The method of claim 44, wherein the AAV vector includes a nucleic acid that encodes NGF or hTERT.

47. The method of claim 38, wherein the microbiome-based therapy includes providing one or more fecal microbiota transplant therapeutic agents to the subject.

48. The method of claim 47, wherein the fecal microbiota transplant therapeutic agents are capsules containing at least one microbiota organism derived or isolated from feces.

49. The method of claim 48, wherein the microbiome-based therapy includes providing a probiotic to the subject.

50. The method of claim 49, wherein the probiotic is Omni-Biotic stress repair.

51. The method of claim 38, wherein the microbiome-based therapy includes providing a sodium oligo-mannurarate remodelling agent to the subject.

52. The method of claim 51, wherein the sodium oligo-mannurarate remodelling agent is GV-971.

53. The method of claim 38, wherein the peptide-based therapy includes IRL-1620 or a peptide derivative or a variant of activated protein kinase C (APC).

54. The method of claim 53, wherein the peptide derivative or variant of APC includes an amino acid sequence which differs from an amino acid sequence of wild-type APC.

55. The method of claim 53 or claim 54, wherein the peptide derivative or variant of APC is 3K3A-APC.

56. The method of any one of claims 38 to 55, wherein the biologic therapy prevents and/or reduces expression of APOE4 or increases the expression of APOE2 or APOE3, promotes expression of one or more neurotrophic factors, prevents and/or reduces an abnormal biome from developing in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents dysregulation/damage and/or death of a neuron, prevent dysregulation and/or damage to the blood brain barrier, and/or reduces and/or prevents insulin resistance.

57. The method of any one of claims 37 to 56, wherein the biologic therapy is provided at a first dosage which is lower than a second dosage that is provided in the absence of the device.

58. The method of any one of claims 37 to 57, wherein the biologic therapy is provided at a first dosing regimen which is less than a second dosing regimen that is provided in the absence of the device.

59. The method of any one of claims 37 to 58, wherein the biologic therapy is provided via a first route which is different than a second route that is provided in the absence of the device.

60. The method of any one of claims 37 to 59, wherein the biologic therapy is provided by administering the biologic therapy to the subject in need thereof.

61. The method of any one of claims 37 to 60, wherein the condition is neurodegeneration.

62. The method of claim 61, wherein neurodegeneration further comprises Alzheimer’s disease, dementia, and/or cognitive impairment.

63. The method of any one of claims 37 to 62, wherein the device has a low-profile state and a deployed state, and when in the deployed state, the sidewall is generally tubular.

64. The method of any one of claims 37 to 63, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

65. The method of claim 64, wherein when positioned in apposition with the blood vessel and a pulse wave travels through the blood vessel, the flexible damping member applies a stress at the first location along a length of the tubular structure.

66. The method of claim 65, wherein, after stress is applied at the first location, at least the portion of the abating substance moves longitudinally and/or radially along a length of the tubular structure.

67. The method of claim 65, wherein, after stress is applied at the first location, at least a portion of the abating substance is configured to move longitudinally and/or radially from a first location within a first deformable portion to a second location within the first deformable portion of the flexible damping member.

68. The method of claim 65, wherein, after stress is applied at the first location, at least the portion of the abating substance is further configured to move longitudinally and/or radially from the first location to a third location within a second deformable portion of the flexible damping member.

69. The method of any one of claims 37 to 68, wherein the inner surface and/or an outer surface has a generally cylindrical shape or an undulating shape that undulates in a longitudinal direction.

70. The method of any one of claims 37 to 69, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

71. The method of any one of claims 37 to 70, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

72. The method of claim 71, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

73. A method for treating and/or preventing one or more effects of a condition in a subject in need thereof, the method comprising:
providing a biologic therapy for treating and/or preventing one or more effects of the condition to the subject in need thereof, wherein the subject has previously been provided a device that treats or slows one or more effects of the condition and was placed in apposition with a blood vessel, the device comprising —
a flexible damping member forming a generally tubular structure having an inner surface and an outer surface, the inner surface formed of a sidewall having one or more at least partially deformable portions, and
an abating substance disposed within the one or more at least partially deformable portions of the sidewall configured to move longitudinally and/or radially within one partially deformable portion in response to pulsatile blood flow within the blood vessel.

74. The method of claim 73, wherein the biologic therapy is a cell-based therapy, a gene-based therapy, a microbiome-based therapy, and/or a peptide-based therapy.

75. The method of claim 74, wherein the cell-based therapy is stem cell therapy or progenitor cell therapy.

76. The method of claim 75, wherein the stem cell therapy is allogenic or autologous human mesenchymal stem cell therapy.

77. The method of claim 75, wherein the stem cell therapy is autologous bone-marrow derived or adipose-derived human mesenchymal stem cell therapy.

78. The method of claim 75, wherein the stem cell therapy is autologous umbilical cord blood-derived or placenta-derived human mesenchymal stem cell therapy.

79. The method of claim 74, wherein the gene-based therapy includes at least one viral vector.

80. The method of claim 79, wherein the at least one viral vector is an adeno-associated viral (AAV) vector.

81. The method of claim 80, wherein the AAV vector includes a nucleic acid that reduces or prevents expression of APOE4.

82. The method of claim 80, wherein the AAV vector includes a nucleic acid that encodes NGF or hTERT.

83. The method of claim 74, wherein the microbiome-based therapy includes providing one or more fecal microbiota transplant therapeutic agents to the subject.

84. The method of claim 83, wherein the fecal microbiota transplant therapeutic agents are capsules containing at least one microbiota organism derived or isolated from feces.

85. The method of claim 74, wherein the microbiome-based therapy includes providing a probiotic to the subject.

86. The method of claim 85, wherein the probiotic is Omni-Biotic stress repair.

87. The method of claim 74, wherein the microbiome-based therapy includes providing a sodium oligo-mannurarate remodelling agent to the subject.

88. The method of claim 87 wherein the sodium oligo-mannurarate remodelling agent is GV-971.

89. The method of claim 74, wherein the peptide-based therapy includes IRL-1620 or a peptide derivative or variant of activated protein kinase C (APC).

90. The method of claim 89, wherein peptide derivative or variant of APC includes an amino acid sequence which differs from an amino acid sequence of wild-type APC.

91. The method of claim 89 or claim 90, wherein the peptide derivative or variant of APC is 3K3A-APC.

92. The method of any one of claims 74 to 91, wherein the biologic therapy prevents and/or reduces expression of APOE4 or increases the expression of APOE2 or APOE3, promotes expression of one or more neurotrophic factors, prevents and/or reduces an abnormal biome in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents or reduces dysregulation/damage and/or death of a neuron, prevents or reduces blood brain barrier dysregulation or permeability/damage, and/or reduces and/or prevents insulin resistance.

93. The method of any one of claims 73 to 89, wherein the biologic therapy is provided at a first dosage which is lower than a second dosage that is provided in the absence of the device.

94. The method of any one of claims 73 to 93, wherein the biologic therapy is provided at a first dosing regimen which is less than a second dosing regimen that is provided in the absence of the device.

95. The method of any one of claims 73 to 94, wherein the biologic therapy is provided via a first route which is different than a second route that is provided in the absence of the device.

96. The method of any one of claims 73 to 95, wherein the biologic therapy is provided by administering the biologic therapy to the subject in need thereof.

97. The method of any one of claims 73 to 96, wherein the condition is neurodegeneration.

98. The method of claim 97, wherein neurodegeneration further comprises Alzheimer’s disease, dementia, and/or cognitive impairment.

99. The method of any one of claims 73 to 98, wherein the device has a low-profile state and a deployed state, and when in the deployed state, the sidewall is generally tubular.

100. The method of any one of claims 73 to 99, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

101. The method of claim 100, wherein when positioned in apposition with the blood vessel and a pulse wave travels through the blood vessel, the flexible damping member applies a stress at the first location along a length of the tubular structure.

102. The method of claim 101, wherein, after stress is applied at the first location, at least the portion of the abating substance moves longitudinally and/or radially along a length of the tubular structure.

103. The method of claim 101, wherein, after stress is applied at the first location, at least a portion of the abating substance is configured to move longitudinally and/or radially from a first location within a first deformable portion to a second location within the first deformable portion of the flexible damping member.

104. The method of claim 101, wherein, after stress is applied at the first location, at least the portion of the abating substance is further configured to move longitudinally and/or radially from the first location to a third location within a second deformable portion of the flexible damping member.

105. The method of any one of claims 73 to 104, wherein the inner surface and/or an outer surface has a generally cylindrical shape or an undulating shape that undulates in a longitudinal direction.

106. The method of any one of claims 73 to 105, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

107. The method of any one of claims 73 to 106, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

108. The method of claim 107, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

109. A system for treating and/or preventing one or more effects of a condition in a subject in need thereof, the system comprising:
an effective amount of a biologic therapy for treating and/or preventing one or more effects of the condition
a device for treating and/or preventing one or more effects of the condition,
the device comprising —
a flexible damping member forming a generally tubular structure having an inner surface and an outer surface, the inner surface formed of a sidewall having one or more at least partially deformable portions, and
an abating substance disposed within the one or more at least partially deformable portions of the sidewall configured to move longitudinally and/or radially within one partially deformable portion in response to pulsatile blood flow within the blood vessel.

110. The system of claim 109, wherein the biologic therapy is a cell-based therapy, a gene-based therapy, a microbiome-based therapy, and/or a peptide-based therapy.

111. The system of claim 110, wherein the cell-based therapy is stem cell therapy or progenitor cell therapy.

112. The system of claim 111, wherein the stem cell therapy is allogenic or autologous human mesenchymal stem cell therapy.

113. The system of claim 111, wherein the stem cell therapy is autologous bone-marrow derived or adipose-derived human mesenchymal stem cell therapy.

114. The system of claim 111, wherein the stem cell therapy is autologous umbilical cord blood-derived or placenta-derived human mesenchymal stem cell therapy.

115. The system of claim 110, wherein the gene-based therapy includes at least one viral vector.

116. The system of claim 115, wherein the at least one viral vector is an adeno-associated viral (AAV) vector.

117. The system of claim 116, wherein the AAV vector includes a nucleic acid that reduces or prevents expression of APOE4.

118. The system of claim 116, wherein the AAV vector includes a nucleic acid that encodes NGF or hTERT.

119. The system of claim 110, wherein the microbiome-based therapy includes providing one or more fecal microbiota transplant therapeutic agents to the subject.

120. The system of claim 119, wherein the fecal microbiota transplant therapeutic agents are capsules containing at least one microbiota organism derived or isolated from feces.

121. The system of claim 110, wherein the microbiome-based therapy includes providing a probiotic to the subject.

122. The system of claim 121, wherein the probiotic is Omni-Biotic stress repair.

123. The system of claim 110, wherein the microbiome-based therapy includes providing a sodium oligo-mannurarate remodelling agent to the subject.

124. The system of claim 123 wherein the sodium oligo-mannurarate remodelling agent is GV-971.

125. The system of claim 110, wherein the peptide-based therapy includes IRL-1620 or a peptide derivative or variant of activated protein kinase C (APC).

126. The system of claim 125, wherein peptide derivative or variant of APC includes an amino acid sequence which differs from an amino acid sequence of wild-type APC.

127. The system of claim 125 or claim 126, wherein the peptide derivative or variant of APC is 3K3A-APC.

128. The system of any one of claims 110 to 127, wherein the biologic therapy prevents and/or reduces expression of APOE4, increases expression of APOE2 or APOE3, promotes expression of one or more neurotrophic factors, prevents and/or reduces an abnormal biome in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation (systemic/blood brain barrier/neuro), reduces and/or prevents oxidative stress, reduces and/or prevents ischemia, prevents or reduces dysregulation/damage and/or death of a neuron, prevents or reduces blood brain barrier dysregulation or permeability/damage, and/or reduces and/or prevents insulin resistance.

129. The system of any one of claims 109 to 125, wherein the biologic therapy is provided at a first dosage which is lower than a second dosage that is provided in the absence of the device.

130. The system of any one of claims 109 to 129, wherein the biologic therapy is provided at a first dosing regimen which is less than a second dosing regimen that is provided in the absence of the device.

131. The system of any one of claims 109 to 130, wherein the biologic therapy is provided via a first route which is different than a second route that is provided in the absence of the device.

132. The system of any one of claims 109 to 131, wherein the biologic therapy is provided by administering the biologic therapy to the subject in need thereof.

133. The system of any one of claims 109 to 132, wherein the condition is neurodegeneration.

134. The system of claim 133, wherein neurodegeneration further comprises Alzheimer’s disease, dementia, and/or cognitive impairment.

135. The system of any one of claims 109 to 134, wherein the inner surface and/or an outer surface has a generally cylindrical shape or an undulating shape that undulates in a longitudinal direction.

136. The system of any one of claims 109 to 135, wherein the device has a low-profile state and a deployed state, and when in the deployed state, the sidewall is generally tubular.

137. The system of any one of claims 109 to 136, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

138. The system of claim 137, wherein when positioned in apposition with the blood vessel and a pulse wave travels through the blood vessel, the flexible damping member applies a stress at the first location along a length of the tubular structure.

139. The system of claim 138, wherein, after stress is applied at the first location, at least the portion of the abating substance moves longitudinally and/or radially along a length of the tubular structure.

140. The system of claim 138, wherein, after stress is applied at the first location, at least a portion of the abating substance is configured to move longitudinally and/or radially from a first location within a first deformable portion to a second location within the first deformable portion of the flexible damping member.

141. The system of claim 138, wherein, after stress is applied at the first location, at least the portion of the abating substance is further configured to move longitudinally and/or radially from the first location to a third location within a second deformable portion of the flexible damping member.

142. The system of any one of claims 109 to 141, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

143. The system of any one of claims 109 to 142, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

144. The system of claim 143, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

145. A system for treating and/or preventing one or more effects of a condition in a subject in need thereof, the system comprising:
an effective amount of a biologic therapy for treating and/or preventing one or more effects of the condition
a device for treating and/or preventing one or more effects of the condition,
the device comprising —
a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
an abating substance disposed within the one or more at least partially deformable portions of the sidewall configured to move longitudinally and/or radially within one partially deformable portion in response to pulsatile blood flow within the blood vessel;
wherein, the effective amount of the biologic therapy for treating and/or preventing one or more effects of the condition is carried by biologic or more of the at least partially deformable portions of the device, and
wherein, when the one or more at least partially deformable portions are at least partially deformed, the effective amount of biologic therapy for treating and/or preventing one or more effects of the condition is released from the device.

146. The system of claim 145, wherein the effective amount of the biologic therapy further comprises a first effective amount of the biologic therapy and a second effective amount of the biologic therapy.

147. The system of claim 146, wherein the second effective amount of the biologic therapy is greater than the first effective amount of the biologic therapy.

148. The system of claim 145, wherein, in response to a first pulsatile blood flow within the blood vessel, the one or more at least partially deformable portions are at least partially deformed to a first degree of deformation.

149. The system of claim 148, wherein, in response to a second pulsatile blood flow within the blood vessel, the one or more at least partially deformable portions are at least partially deformed to a second degree of deformation.

150. The system of claim 149, wherein the second degree of deformation is greater than the first degree of deformation.

151. The system of claim 150, wherein the first effective amount of the biologic therapy is released from the one or more at least partially deformable portions in response to the first degree of deformation.

152. The system of claim 150, wherein the second effective amount of the biologic therapy is released from the one or more at least partially deformable portions in response to the second degree of deformation.

153. The system of any one of claims 145 to 152, wherein the biologic therapy is a cell-based therapy, a gene-based therapy, a microbiome-based therapy, and/or a peptide-based therapy.

154. The system of claim 153, wherein the cell-based therapy is stem cell therapy or progenitor cell therapy.

155. The system of claim 154, wherein the stem cell therapy is allogenic or autologous human mesenchymal stem cell therapy.

156. The system of claim 154, wherein the stem cell therapy is autologous bone-marrow derived or adipose-derived human mesenchymal stem cell therapy.

157. The system of claim 154, wherein the stem cell therapy is autologous umbilical cord blood-derived or placenta-derived human mesenchymal stem cell therapy.

158. The system of claim 153, wherein the gene-based therapy includes at least one viral vector.

159. The system of claim 158, wherein the at least one viral vector is an adeno-associated viral (AAV) vector.

160. The system of claim 159, wherein the AAV vector includes a nucleic acid that reduces or prevents expression of APOE4.

161. The system of claim 159, wherein the AAV vector includes a nucleic acid that encodes NGF or hTERT.

162. The system of claim 153, wherein the microbiome-based therapy includes providing one or more fecal microbiota transplant therapeutic agents to the subject.

163. The system of claim 162, wherein the fecal microbiota transplant therapeutic agents are capsules containing at least one microbiota organism derived or isolated from feces.

164. The system of claim 153, wherein the microbiome-based therapy includes providing a probiotic to the subject.

165. The system of claim 164, wherein the probiotic is Omni-Biotic stress repair.

166. The system of claim 153, wherein the microbiome-based therapy includes providing a sodium oligo-mannurarate remodelling agent to the subject.

167. The system of claim 166 wherein the sodium oligo-mannurarate remodelling agent is GV-971.

168. The system of claim 153, wherein the peptide-based therapy includes IRL-1620 or a peptide derivative or variant of activated protein kinase C (APC).

169. The system of claim 168, wherein peptide derivative or variant of APC includes an amino acid sequence which differs from an amino acid sequence of wild-type APC.

170. The system of claim 168 or claim 169, wherein the peptide derivative or variant of APC is 3K3A-APC.

171. The system of any one of claims 153 to 170, wherein the biologic therapy prevents and/or reduces expression of APOE4, increases expression of APOE2 or APOE3, promotes expression of one or more neurotrophic factors, prevents and/or reduces an abnormal biome in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation (systemic/blood brain barrier/neuro), reduces and/or prevents oxidative stress, reduces and/or prevents ischemia, prevents or reduces dysregulation/damage and/or death of a neuron, prevents or reduces blood brain barrier dysregulation or permeability/damage, and/or reduces and/or prevents insulin resistance.

172. The system of any one of claims 145 to 168, wherein the therapy is provided at a first dosage which is lower than a second dosage that is provided in the absence of the device.

173. The system of any one of claims 145 to 172, wherein the therapy is provided at a first dosing regimen which is less than a second dosing regimen that is provided in the absence of the device.

174. The system of any one of claims 145 to 173, wherein the therapy is provided via a first route which is different than a second route that is provided in the absence of the device.

175. The system of any one of claims 145 to 174, wherein the therapy is provided by administering the therapy to the subject in need thereof.

176. The system of any one of claims 145 to 175, wherein the condition is neurodegeneration.

177. The system of claim 176, wherein neurodegeneration further comprises Alzheimer’s disease, dementia, and/or cognitive impairment.

178. The system of any one of claims 145 to 177, wherein the inner surface and/or an outer surface has a generally cylindrical shape or an undulating shape that undulates in a longitudinal direction.

179. The system of any one of claims 145 to 173, wherein the device has a low-profile state and a deployed state, and when in the deployed state, the sidewall is generally tubular.

180. The system of any one of claims 145 to 179, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

181. The system of claim 180, wherein when positioned in apposition with the blood vessel and a pulse wave travels through the blood vessel, the flexible damping member applies a stress at the first location along a length of the tubular structure.

182. The system of claim 181, wherein, after stress is applied at the first location, at least the portion of the abating substance moves longitudinally and/or radially along a length of the tubular structure.

183. The system of claim 181, wherein, after stress is applied at the first location, at least a portion of the abating substance is configured to move longitudinally and/or radially from a first location within a first deformable portion to a second location within the first deformable portion of the flexible damping member.

184. The system of claim 181, wherein, after stress is applied at the first location, at least the portion of the abating substance is further configured to move longitudinally and/or radially from the first location to a third location within a second deformable portion of the flexible damping member.

185. The system of any one of claims 145 to 184, wherein the flexible damping member is further configured to be positioned around at least a portion of a circumference of a wall of the blood vessel and a pulse wave traveling through the blood vessel applies a stress at a first region of the damping member, at least a portion of the abating substance moves away from the first region to a second region of the damping member such that the damping member absorbs at least a portion of the energy of the pulse wave, thereby reducing the stress on the blood vessel wall distal to the device.

186. The system of any one of claims 145 to 185, wherein the device is further configured to be deployed within a lumen of the blood vessel such that an outer surface of an anchoring member is in apposition with a lumen of the blood vessel wall and the outer surface of the sidewall is in contact with blood flowing through the blood vessel lumen.

187. The system of claim 186, wherein when the device is deployed within the blood vessel lumen and a pulse wave traveling through the blood vessel applies a stress at a third location of the damping member, at least a portion of the abating substance moves away from the third location to a fourth location of the damping member such that the damping member absorbs at least a portion of the energy of the pulse wave, thereby reducing the stress on the blood vessel wall distal to the device.

188. A method for treating a patient having a condition comprising:
(a) determining or having determined whether the patient has an elevated pulse pressure or elevated pulse wave intensity/FCWI and a history of a microbleed, blood brain barrier dysfunction or permeability, systemic inflammation, blood brain barrier inflammation, neuroinflammation, increased level of at least one circulating or CSF cytokine, increased reactive oxygen species, or a combination thereof by:
(i) obtaining or having obtained information which indicates that the patient has the elevated pulse pressure or elevated pulse wave intensity/FCWI and has previously had symptoms of a microbleed, blood brain barrier dysfunction or permeability, systemic inflammation, blood brain barrier inflammation, neuroinflammation, increased level of at least one circulating cytokine, increased reactive oxygen species, or a combination thereof and/or was previously diagnosed with a microbleed, blood brain barrier dysfunction or permeability, systemic inflammation, blood brain barrier inflammation, neuroinflammation, increased level of at least one circulating cytokine, increased reactive oxygen species or a combination thereof; and/or
(ii) monitoring or having monitored the subject for the elevated pulse pressure and symptoms of a microbleed, blood brain barrier dysfunction or permeability, systemic inflammation, blood brain barrier inflammation, neuroinflammation, increased level of at least one circulating cytokine, increased reactive oxygen species or a combination thereof; and
(b) if the patient has previously had symptoms of a microbleed, blood brain barrier dysfunction or permeability, systemic inflammation, blood brain barrier inflammation, neuroinflammation, increased level of at least one circulating or CSF cytokine, increased reactive oxygen species, or a combination thereof, was previously diagnosed with a microbleed, blood brain barrier dysfunction or permeability, systemic inflammation, blood brain barrier inflammation, neuroinflammation, increased level of at least one circulating cytokine, increased reactive oxygen species, or a combination thereof, and/or symptoms of a microbleed, blood brain barrier dysfunction or permeability, systemic inflammation, blood brain barrier inflammation, neuroinflammation, increased level of at least one circulating cytokine, increased reactive oxygen species, or a combination thereof were monitored, then
(i) providing a cell-based therapy, a gene-based therapy, a microbiome-based therapy, or a peptide-based therapy to the patient, and
(ii) providing a device for treating and/or preventing one or more effects of the condition, the device comprising —
a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
an abating substance disposed within the one or more at least partially deformable portions of the sidewall configured to move longitudinally and/or radially within one partially deformable portion in response to pulsatile blood flow within the blood vessel;
(c) if the patient has not had symptoms of a microbleed, APOE4 expression, blood brain barrier dysfunction or permeability, systemic inflammation, blood brain barrier inflammation, neuroinflammation, increased level of at least one circulating or CSF cytokine, increased reactive oxygen species, or a combination thereof, was not previously diagnosed with a microbleed, APOE4 expression, blood brain barrier dysfunction or permeability, systemic inflammation, blood brain barrier inflammation, neuroinflammation, increased level of at least one circulating or CSF cytokine, increased reactive oxygen species, or a combination thereof, then providing the device for treating and/or preventing one or more effects of the condition.

189. The method of claim 188, wherein the elevated pulse pressure is a pulse pressure of at least 50 mmHg.

190. The method of claim 188 or claim 189, wherein the increased level of at least one circulating cytokine (b) is a level at least about 5% greater than the level in (c).

191. The method of any one of claims 188 to 190, wherein the at least one cytokine is selected from the group consisting of VCAM-1, ICAM-1, TNFα, TGF-β, IL-6, IL-8, IL-10, IL-12, and NF-κB.

192. The method of claim 188, wherein the cell-based therapy is stem cell therapy or progenitor cell therapy.

193. The method of claim 192, wherein the stem cell therapy is allogenic or autologous human mesenchymal stem cell therapy.

194. The method of claim 192, wherein the stem cell therapy is autologous bone-marrow derived or adipose-derived human mesenchymal stem cell therapy.

195. The method of claim 192, wherein the stem cell therapy is autologous umbilical cord blood-derived or placenta-derived human mesenchymal stem cell therapy.

196. The method of claim 188, wherein the gene-based therapy includes at least one viral vector.

197. The method of claim 196, wherein at least one viral vector is an adeno-associated viral (AAV) vector.

198. The method of claim 197, wherein the AAV vector includes a nucleic acid that reduces or prevents expression of APOE4.

199. The method of claim 197, wherein the AAV vector includes a nucleic acid that encodes NGF or hTERT.

200. The method of claim 188, wherein the microbiome-based therapy includes providing one or more fecal microbiota transplant therapeutic agents to the subject.

201. The method of claim 200, wherein the fecal microbiota transplant therapeutic agents are capsules containing at least one microbiota organism derived or isolated from feces.

202. The method of claim 188, wherein the microbiome-based therapy includes providing a probiotic, to the subject.

203. The method of claim 202, wherein the probiotic is Omni-Biotic stress repair.

204. The method of claim 188, wherein the microbiome-based therapy includes providing a sodium oligo-mannurarate remodelling agent to the subject.

205. The method of claim 204 wherein the sodium oligo-mannurarate remodelling agent is GV-971.

206. The method of claim 188 wherein the peptide-based therapy includes IRL-1620 or a peptide derivative of APC.

207. The method of claim 206, wherein the peptide derivative or variant of APC includes an amino acid sequence which differs from an amino acid sequence of wild-type APC.

208. The method of claim 206 or claim 207, wherein the peptide derivative or variant of APC is 3K3A-APC.

209. The method of any one of claims 188 to 208, wherein the therapy prevents and/or reduces expression of APOE4, increases expression of APOE2 or APOE3, promotes expression of one or more neurotrophic factors, prevents and/or reduces an abnormal biome from developing in the subject, prevents and/or reduces abnormal cleavage of amyloid precursor protein in the subject’s brain, prevents and/or reduces expression and/or accumulation of β-amyloid protein in the subject’s brain, prevents and/or reduces expression and/or accumulation of tau protein in the subject’s brain, increases neurotransmission, decreases inflammation, reduces and/or prevents oxidative stress, reduces and/or prevents ischemia, prevents damage and/or death of a neuron, and/or reduces and/or prevents insulin resistance.

210. The method of any one of claims 188 to 209, wherein the therapy is provided at a first dosage which is lower than a second dosage that is provided in the absence of the device.

211. The method of any one of claims 188 to 210, wherein the therapy is provided at a first dosing regimen which is less than a second dosing regimen that is provided in the absence of the device.

212. The method of any one of claims 188 to 211, wherein the therapy is provided via a first route which is different than a second route that is provided in the absence of the device.

213. The method of any one of claims 188 to 212, wherein the therapy is provided by administering the therapy to the subject in need thereof.

214. The method of any one of claims 188 to 213, wherein step (b) is performed after step (a) and before step (c).

215. The method of any one of claims 188 to 214, wherein step (c) is performed after step (a) and before step (b).

216. The method of any one of claims 188 to 213, wherein the condition is neurodegeneration.

217. The method of claim 216, wherein neurodegeneration further comprises Alzheimer’s disease, dementia, and/or cognitive impairment.

218. The method of any one of claims 188 to 217, wherein the inner surface and/or an outer surface has a generally cylindrical shape or an undulating shape that undulates in a longitudinal direction.

219. The method of any one of claims 188 to 218, wherein the device has a low-profile state and a deployed state, and when in the deployed state, the sidewall is generally tubular.

220. The method of any one of claims 188 to 219, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

221. A method for treating a patient having a condition comprising:
(a) determining or having determined whether the patient has an elevated pulse pressure or elevated pulse wave intensity/FCWI and a history of a reduced level of one or more circulating neurotrophic factors:
(i) obtaining or having obtained information which indicates that the patient has the elevated pulse pressure or elevated pulse wave intensity/FCWI and the reduced level of one or more circulating neurotrophic factors and/or was previously diagnosed with the reduced level of one or more circulating neurotrophic factors; and/or
(ii) monitoring or having monitored the subject for the elevated pulse pressure or elevated pulse wave intensity/FCWI and the reduced level of one or more circulating neurotrophic factors; and
(b) if the patient has previously had the reduced level of one or more circulating neurotrophic factors, was previously diagnosed with the reduced level of one or more circulating neurotrophic factors, and/or the reduced level of one or more circulating neurotrophic factors was monitored, then
(i) providing a cell-based therapy to the patient, and
(ii) providing a device for treating and/or preventing one or more effects of the condition, the device comprising —
a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
an abating substance disposed within the one or more at least partially deformable portions of the sidewall configured to move longitudinally and/or radially within one partially deformable portion in response to pulsatile blood flow within the blood vessel;
(c) if the patient has not had the reduced level of one or more circulating neurotrophic factors, was not previously diagnosed with the reduced level of one or more circulating neurotrophic factors, then providing the device for treating and/or preventing one or more effects of the condition.

222. The method of claim 221, wherein the elevated pulse pressure is a pulse pressure of at least 50 mmHg.

223. The method of claim 221 or claim 222, wherein the reduced level of one or more circulating neurotrophic factors in (b) is a level of at least about 5% greater than the level in (c).

224. The method of any one of claims 221 to 223, wherein the one or more circulating neurotrophic factors is selected from the group consisting of NGF and BDNF.

225. The method of claim 221, wherein the cell-based therapy is stem cell therapy or progenitor cell therapy.

226. The method of claim 225, wherein the stem cell therapy is allogenic or autologous human mesenchymal stem cell therapy.

227. The method of claim 225, wherein the stem cell therapy is autologous bone-marrow derived or adipose-derived human mesenchymal stem cell therapy.

228. The method of claim 225, wherein the stem cell therapy is autologous umbilical cord blood-derived or placenta-derived human mesenchymal stem cell therapy.

229. The method of any one of claims 221 to 228, wherein the cell-based therapy promotes expression of one or more neurotrophic factors.

230. The method of any one of claims 221 to 229, wherein the cell-based therapy is provided at a first dosage which is lower than a second dosage that is provided in the absence of the device.

231. The method of any one of claims 221 to 230, wherein the cell-based therapy is provided at a first dosing regimen which is less than a second dosing regimen that is provided in the absence of the device.

232. The method of any one of claims 221 to 231, wherein the cell-based therapy is provided via a first route which is different than a second route that is provided in the absence of the device.

233. The method of any one of claims 221 to 232, wherein the cell-based therapy is provided by administering the cell-based therapy to the subject in need thereof.

234. The method of any one of claims 221 to 233, wherein step (b) is performed after step (a) and before step (c).

235. The method of any one of claims 221 to 234, wherein step (c) is performed after step (a) and before step (b).

236. The method of any one of claims 221 to 233, wherein the condition is neurodegeneration.

237. The method of claim 236, wherein neurodegeneration further comprises Alzheimer’s disease, dementia, and/or cognitive impairment.

238. The method of any one of claims 221 to 237, wherein the inner surface and/or an outer surface has a generally cylindrical shape or an undulating shape that undulates in a longitudinal direction.

239. The method of any one of claims 221 to 238, wherein the device has a low-profile state and a deployed state, and when in the deployed state, the sidewall is generally tubular.

240. The method of any one of claims 221 to 239, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.

241. A method for treating a patient having a condition comprising:
(a) determining or having determined whether the patient has an elevated pulse pressure or an elevated pulse wave intensity/FCWI and an expression of APOE4 by:
(i) obtaining or having obtained information which indicates that the patient has the elevated pulse pressure or an elevated pulse wave intensity/FCWI and has previously had the expression of APOE4 and/or was previously diagnosed with the expression of APOE4; and/or
(ii) monitoring or having monitored the subject for the elevated pulse pressure or an elevated pulse wave intensity/FCWI and the expression of APOE4; and
(b) if the patient has previously had the expression of APOE4, was previously diagnosed with the expression of APOE4, and/or the expression of APOE4 was monitored, then
(i) providing a gene-based therapy to the patient, and
(ii) providing a device for treating and/or preventing one or more effects of the condition, the device comprising —
a flexible damping member forming a generally tubular structure having an inner surface and an outer surface, the inner surface formed of a sidewall having one or more at least partially deformable portions configured to move longitudinally and/or radially within the one or more at least partially deformable portions in response to pulsatile blood flow within the blood vessel, and
an abating substance disposed within the one or more at least partially deformable portions of the sidewall configured to move longitudinally and/or radially within one partially deformable portion in response to pulsatile blood flow within the blood vessel;
(c) if the patient has not had symptoms of the expression of APOE4, was not previously diagnosed with the expression of APOE4, then providing the device for treating and/or preventing one or more effects of the condition.

242. The method of claim 241, wherein the elevated pulse pressure is a pulse pressure of at least 50 mmHg.

243. The method of claim 241 or claim 242, wherein the expression of APOE4 in (b) is a level of at least about 5% greater than the level in (c).

244. The method of claim 241, wherein the gene-based therapy includes at least one viral vector.

245. The method of claim 244, wherein at least one viral vector is an adeno-associated viral (AAV) vector.

246. The method of claim 245, wherein the AAV vector includes a nucleic acid that reduces or prevents expression of APOE4.

247. The method of any one of claims 241 to 246, wherein the gene-based therapy prevents and/or reduces expression of APOE4 or increases the expression of APOE2 or APOE3.

248. The method of any one of claims 241 to 247, wherein the gene-based therapy is provided at a first dosage which is lower than a second dosage that is provided in the absence of the device.

249. The method of any one of claims 241 to 248, wherein the gene-based therapy is provided at a first dosing regimen which is less than a second dosing regimen that is provided in the absence of the device.

250. The method of any one of claims 241 to 249, wherein the gene-based therapy is provided via a first route which is different than a second route that is provided in the absence of the device.

251. The method of any one of claims 241 to 250, wherein the gene-based therapy is provided by administering the therapy to the subject in need thereof.

252. The method of any one of claims 241 to 251, wherein step (b) is performed after step (a) and before step (c).

253. The method of any one of claims 241 to 252, wherein step (c) is performed after step (a) and before step (b).

254. The method of any one of claims 241 to 253, wherein the condition is neurodegeneration.

255. The method of claim 254, wherein neurodegeneration further comprises Alzheimer’s disease, dementia, and/or cognitive impairment.

256. The method of any one of claims 241 to 255, wherein the inner surface and/or an outer surface has a generally cylindrical shape or an undulating shape that undulates in a longitudinal direction.

257. The method of any one of claims 241 to 256, wherein the device has a low-profile state and a deployed state, and when in the deployed state, the sidewall is generally tubular.

258. The method of any one of claims 241 to 257, wherein the abating substance is configured to expand in response to an increase of blood/pulse pressure or elevated pulse wave intensity/FCWI within the blood vessel and relax as the blood/pulse pressure or elevated pulse wave intensity/FCWI within the blood vessel subsequently decreases.
